(19) United States
(12) Patent Application Publication
(10) Pub. No.: US 2012/0149694 A1
(43) Pub. Date: Jun. 14, 2012

(54) Substituted piperidines

(75) Inventors: Dirk Heimbach, Dusseldorf (DE); Susanne Röhrig, Hilden (DE); Yolanda Cancho Grande, Leverkusen (DE); Ulrich Rester, Wuppertal (DE); Eckhard Bender, Langenfeld (DE); Katja Zimmermann, Dusseldorf (DE); Dmitry Zubov, Remscheid (DE); Anja Buchmüller, Essen (DE); Georges Von Degenfeld, Leverkusen (DE); Christoph Gerdes, Koln (DE); Mark Jean Gnoth, Mettmann (DE); Kersten Matthias Gericke, Wuppertal (DE); Mario Jeske, Solingen (DE)

(73) Assignee: BAYER PHARMA AKTIENGESELLSCHAFT, Berlin (DE)

(21) Appl. No.: 13/321,790

(22) PCT Filed: May 14, 2010
(86) PCT No.: PCT/EP2010/002967

§ 371 Date: Feb. 27, 2012

(30) Foreign Application Priority Data
May 27, 2009 (DE) 102009022892.6

(51) Int. Cl. A61K 31/541; C07D 413/14; A61P 35/00; A61K 31/5377; A61P 7/02; A61P 9/00; C07D 413/06; C07D 417/06

(52) U.S. Cl. 514/227.8; 544/130; 544/58.2; 514/235.5

(57) ABSTRACT

The invention relates to novel substituted piperidines, to processes for preparation thereof, to the use thereof for treatment and/or prophylaxis of diseases and to the use thereof for production of medicaments for treatment and/or prophylaxis of diseases, especially of cardiovascular disorders and tumour disorders.

The invention relates to novel substituted piperidines, to processes for preparation thereof, to the use thereof for treatment and/or prophylaxis of diseases and to the use thereof for production of medicaments for treatment and/or prophylaxis of diseases, especially of cardiovascular disorders and tumour disorders.

Thrombocytes (blood platelets) are a significant factor both in physiological haemostasis and in thromboembolic disorders. In the arterial system in particular, platelets are of central importance in the complex interaction between blood components and the wall of the vessel. Unwanted platelet activation may, through formation of platelet-rich thrombi, result in thromboembolic disorders and thrombotic complications with life-threatening conditions.

One of the most potent platelet activators is the blood coagulation protease thrombin, which is formed at injured blood vessel walls and which, in addition to fibrin formation, leads to the activation of platelets, endothelial cells and mesenchymal cells (Vu T K H, Hung D T, Wheaton V I, Coughlin S R, Cell 1991, 64, 1057-1068). In platelets in vitro and in animal models, thrombin inhibitors inhibit platelet aggregation and the formation of platelet-rich thrombi. In man, arterial thromboses can be prevented or treated successfully with inhibitors of platelet function and thrombin inhibitors (Bhatt D L, Topol E J, Nat. Rev. Drug Discov. 2003, 2, 15-28). Therefore, there is a high probability that antagonists of thrombin action on platelets will reduce the formation of thrombi and the occurrence of clinical sequelae such as myocardial infarction and stroke. Other cellular effects of thrombin, for example on endothelial cells and smooth-muscle cells of vessels, on leukocytes and on fibroblasts, are possibly responsible for inflammatory and proliferative disorders.

At least some of the cellular effects of thrombin are mediated via a family of G-protein-coupled receptors (Protease Activated Receptors, PARs), the prototype of which is the PAR-1 receptor. PAR-1 is activated by binding of thrombin and proteolytic cleavage of its extracellular N-terminus. The proteolysis exposes a new N-terminus having the amino acid sequence SFLLRN . . . , which, as an agonist (“tethered ligand”) leads to intramolecular receptor activation and transmission of intracellular signals. Peptides derived from the tethered-ligand sequence can be used as agonists of the receptor and, on platelets, lead to activation and aggregation. Other proteases are likewise capable of activating PAR-1, including, for example, plasmin, factor VIIa, factor Xa, trypsin, activated protein C (aPC), tryptase, cathepsin G, proteinase 3, granzyme A, elastase and matrix metalloprotease 1 (MMP-1).

In contrast to the inhibition of protease activity of thrombin with direct thrombin inhibitors, blockade of PAR-1 should result in an inhibition of platelet activation without reduction of the coagulability of the blood (anticoagulation).

Antibodies and other selective PAR-1 antagonists inhibit the thrombin-induced aggregation of platelets in vitro at low to medium thrombin concentrations (Kahn M L, Nakanishi-Matsui M, Shapiro M J, Ishihara H, Coughlin S R, J. Clin. Invest. 1999, 103, 879-887). A further thrombin receptor with possible significance for the pathophysiology of thrombotic processes, PAR-4, was identified on human and animal platelets. In experimental thromboses in animals having a PAR expression pattern comparable to humans, PAR-1 antagonists reduce the formation of platelet-rich thrombi (Derian C K, Damiano B P, Addo M F, Darrow A L, D'Andrea M R, Nedelman M, Zhang H-C, Maryanoff B E, Andrade-Gordon P, J. Pharmacol. Exp. Ther. 2003, 304, 855-861).

In the last few years, a large number of substances have been examined for their platelet function-inhibiting action; but only a few platelet function inhibitors have been found to be useful in practice. There is therefore a need for pharmaceuticals which specifically inhibit an increased platelet reaction without significantly increasing the risk of bleeding, and hence reduce the risk of thromboembolic complications.

Effects of thrombin which are mediated via the PAR-1 receptor affect the progression of disease during and after coronary artery bypass graft (CABG) and other operations and especially operations with extracorporeal circulation (for example heart-lung machine). During the course of the operation, there may be bleeding complications owing to pre- or intraoperative medication with coagulation-inhibiting and/or platelet-inhibiting substances. For this reason, for example, medication with clopidogrel has to be interrupted several days prior to a CABG. Moreover, as mentioned, disseminated intravascular coagulation or consumption coagulopathy (DIC) may develop (for example owing to the extended contact between blood and synthetic surfaces in the case of use of extracorporeal circulation or during blood transfusions), which in turn can lead to bleeding complications. Later, there is frequently restenosis of the venous or arterial bypasses grafted (which may even result in occlusion) owing to thrombosis, intimafibrosis, arteriosclerosis, angina pectoris, myocardial infarction, heart failure, arrhythmias, transitory ischaemic attack (TIA) and/or stroke.

In man, the PAR-1 receptor is also expressed in other cells including, for example, endothelial cells, smooth muscle cells and tumour cells. Malignant tumour disorders (cancer) have a high incidence and are generally associated with high mortality. Current therapies achieve full remission in only a fraction of patients and are typically associated with severe side effects. There is therefore a great need for more effective and safer therapies. The PAR-1 receptor contributes to cancer generation, growth, invasiveness and metastasis. Moreover, PAR-1 expressed on endothelial cells mediates signals resulting in vascular growth (“angiogenesis”), a process which is vital for allowing a tumour to grow larger than about 1 mm³ Angiogenesis also contributes to the genesis or worsening of other disorders including, for example, haematopoetic cancer disorders, macular degeneration, which leads to blindness, and diabetic retinopathy, inflammatory disorders, such as rheumatoid arthritis and colitis.

Sepsis (or septicaemia) is a frequent disorder with high mortality. Initial symptoms of sepsis are typically unspecific (for example fever, reduced general state of health); however, there may later be generalized activation of the coagulation system (“disseminated intravascular coagulation” or “consumption coagulopathy” (DIC)) with the formation of microthrombi in various organs and secondary bleeding complications. DIC may also occur independently of a sepsis, for example in the course of operations or in the event of tumour disorders.

Treatment of sepsis consists firstly in the rigorous elimination of the infectious cause, for example by operative removal of the focus and antibiosis. Secondly, it consists in temporary intensive medical support of the affected organ systems. Treatments of the different stages of this disease have been described, for example, in the following publication (Dellinger et al., Crit. Care Med. 2004, 32, 858-873). There are no proven effective treatments for DIC.

It is therefore an object of the present invention to provide novel PAR-1 antagonists for treatment of disorders, for example cardiovascular disorders and thromboembolic disorders, and also tumour disorders, in humans and animals.

WO 2006/002349, WO 2006/002350, WO 2007/089683 and WO 2007/101270 describe structurally similar piperidines as 11-β HSD1 inhibitors for treatment of diabetes, thromboembolic disorders and stroke, among other disorders.

The invention provides compounds of the formula

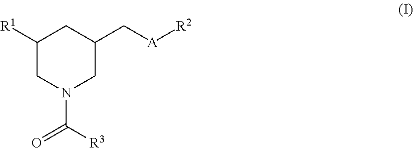

in which
A is an oxygen atom, a sulphur atom, —NR⁴—, —S(═O)— or —S(═O)₂—,
   where
   R⁴ is hydrogen or C₁-C₃-alkyl, or
   R² and R⁴ together with the nitrogen atom to which they are bonded form a 5- or 6-membered heterocycle,
      in which the heterocycle may be substituted by 1 to 3 substituents selected independently from the group consisting of halogen, cyano, hydroxyl, amino, C₁-C₄-alkyl, C₁-C₄-alkoxy and C₁-C₄-alkylamino, or
A is a group of the formula

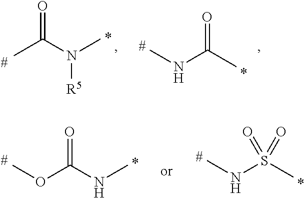

where
# is the attachment site to the piperidine ring,
* is the attachment site of R², and
R⁵ is hydrogen or C₁-C₃-alkyl,
R¹ is phenyl,
   where phenyl may be substituted by 1 to 3 substituents selected independently from the group consisting of monofluoromethyl, difluoromethyl, trifluoromethyl, monofluoromethoxy, difluoromethoxy, trifluoromethoxy, monofluoromethylsulphanyl, difluoromethylsulphanyl, trifluoromethylsulphanyl, methylsulphonyl, C₁-C₄-alkyl, C₁-C₄-alkoxy and C₁-C₄-alkoxycarbonyl,
R² is C₁-C₆-alkyl, C₃-C₆-cycloalkyl, 4- to 6-membered heterocyclyl, phenyl or 5- or 6-membered heteroaryl,
   where cycloalkyl, heterocyclyl, phenyl and heteroaryl may be substituted by 1 to 3 substituents selected independently from the group consisting of halogen, cyano, hydroxyl, amino, monofluoromethyl, difluoromethyl, trifluoromethyl, monofluoromethoxy, difluoromethoxy, trifluoromethoxy, monofluoromethylsulphanyl, difluoromethylsulphanyl, trifluoromethylsulphanyl, C₁-C₄-alkyl, C₁-C₄-alkoxy, C₁-C₆-alkylamino and phenyl,
      in which phenyl may be substituted by 1 to 3 substituents selected independently from the group consisting of halogen and trifluoromethyl, and
   where C₁-C₆-alkyl may be substituted by one substituent selected from the group consisting of C₁-C₄-alkoxy, C₁-C₄-alkylsulphonyl, C₃-C₆-cycloalkyl and phenyl,
      in which cycloalkyl and phenyl may be substituted by 1 to 3 substituents selected independently from the group consisting of halogen, cyano, monofluoromethyl, difluoromethyl, trifluoromethyl, monofluoromethoxy, difluoromethoxy, trifluoromethoxy, C₁-C₄-alkyl and C₁-C₄-alkoxy,
R³ is C₁-C₆-alkyl, C₁-C₆-alkoxy, C₁-C₆-alkylamino, C₃-C₇-cycloalkyl, 4- to 7-membered heterocyclyl, phenyl, 5- or 6-membered heteroaryl, C₃-C₇-cycloalkyloxy, C₃-C₇-cycloalkylamino, 4- to 7-membered heterocyclylamino, phenylamino or 5- or 6-membered heteroarylamino,
   where alkyl, C₂-C₆-alkoxy and alkylamino may be substituted by one substituent selected from the group consisting of halogen, hydroxyl, amino, cyano, C₁-C₄-alkoxy, C₁-C₄-alkoxycarbonyl, C₃-C₇-cycloalkyl, 4- to 6-membered heterocyclyl, phenyl and 5- or 6-membered heteroaryl, and
   where cycloalkyl, heterocyclyl, phenyl, heteroaryl, cycloalkyloxy, cycloalkylamino, heterocyclylamino, phenylamino and heteroarylamino may be substituted by 1 to 3 substituents selected independently from the group consisting of halogen, cyano, oxo, hydroxyl, amino, monofluoromethyl, difluoromethyl, trifluoromethyl, monofluoromethoxy, difluoromethoxy, trifluoromethoxy, mono fluoromethylsulphanyl, difluoromethylsulphanyl, trifluoromethylsulphanyl, hydroxycarbonyl, aminocarbonyl, C₁-C₄-alkyl, C₁-C₄-alkoxy, C₁-C₆-alkylamino, C₁-C₄-alkoxycarbonyl, C₁-C₄-alkylaminocarbonyl and cyclopropyl,
      in which alkyl may be substituted by one hydroxyl substituent,
         and their salts, their solvates and the solvates of their salts.

Inventive compounds are the compounds of the formula (I) and their salts, solvates and solvates of the salts; the compounds, encompassed by formula (I), of the formulae below and their salts, solvates and solvates of the salts, and the compounds encompassed by formula (I) specified below as working examples and their salts, solvates and solvates of the salts, if the compounds, encompassed by formula (I), below are not already salts, solvates and solvates of the salts.

Depending on their structure, the inventive compounds may exist in stereoisomeric forms (enantiomers, diastereomers). The invention therefore encompasses the enantiomers or diastereomers and their respective mixtures. It is possible to isolate the stereoisomerically uniform constituents in a known manner from such mixtures of enantiomers and/or diastereomers.

If the inventive compounds can occur in tautomeric forms, the present invention encompasses all tautomeric forms.

In the context of the present invention, preferred salts are physiologically acceptable salts of the inventive compounds. However, also encompassed are salts which themselves are not suitable for pharmaceutical applications, but which can be used, for example, for the isolation or purification of the inventive compounds.

Physiologically acceptable salts of the inventive compounds include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, maleic acid, citric acid, fumaric acid, maleic acid and benzoic acid.

Physiologically acceptable salts of the inventive compounds also include salts of customary bases, such as, by way of example and with preference, alkali metal salts (for example sodium salts and potassium salts), alkaline earth metal salts (for example calcium salts and magnesium salts) and ammonium salts derived from ammonia or organic amines having 1 to 16 carbon atoms, such as, by way of example and with preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

In the context of the invention, solvates are those forms of the inventive compounds which, in the solid or liquid state, form a complex by coordination with solvent molecules. Hydrates are a specific form of the solvates in which the coordination is with water.

Moreover, the present invention also encompasses prodrugs of the inventive compounds. The term “prodrugs” encompasses compounds which themselves may be biologically active or inactive but which, during their residence time in the body, are converted to inventive compounds (for example metabolically or hydrolytically).

In the context of the present invention, unless specified otherwise, the substituents are defined as follows:

Alkyl per se and “alk” and “alkyl” in alkoxy, alkylamino, alkylcarbonyl, alkylaminocarbonyl and alkylsulphonyl are a straight-chain or branched alkyl radical having 1 to 6 carbon atoms, by way of example and with preference methyl, ethyl, n-propyl, isopropyl, n-butyl, tert-butyl, n-pentyl and n-hexyl.

By way of example and with preference, alkoxy is methoxy, ethoxy, n-propoxy, isopropoxy, n-butoxy, tert-butoxy, n-pentoxy and n-hexoxy.

Alkylamino is an alkylamino radical having one or two (independently selected) alkyl substituents, by way of example and with preference methylamino, ethylamino, n-propylamino, isopropylamino, tert-butylamino, N,N-dimethylamino, N,N-diethylamino, N-ethyl-N-methylamino, N-methyl-N-n-propylamino, N-isopropyl-N-n-propylamino and N-tert-butyl-N-methylamino C₁-C₄-Alkylamino is, for example, a monoalkylamino radical having 1 to 4 carbon atoms or is a dialkylamino radical having in each case 1 to 4 carbon atoms per alkyl substituent.

By way of example and with preference, alkoxycarbonyl is methoxycarbonyl, ethoxycarbonyl, n-propoxycarbonyl, isopropoxycarbonyl, n-butoxycarbonyl and tert-butoxycarbonyl.

Alkylaminocarbonyl is an alkylaminocarbonyl radical having one or two (independently selected) alkyl substituents, by way of example and with preference methylaminocarbonyl, ethylaminocarbonyl, n-propylaminocarbonyl, isopropylaminocarbonyl, tert-butylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl and N-tert-butyl-N-methylaminocarbonyl. C₁-C₄-Alkylaminocarbonyl is, for example, a monoalkylaminocarbonyl radical having 1 to 4 carbon atoms or is a dialkylaminocarbonyl radical having in each case 1 to 4 carbon atoms per alkyl substituent.

By way of example and with preference, alkylsulphonyl is methylsulphonyl, ethylsulphonyl, n-propylsulphonyl, isopropylsulphonyl, n-butylsulphonyl and tert-butylsulphonyl.

Cycloalkyl is a monocyclic cycloalkyl group having generally 3 to 7, preferably 5 or 6, carbon atoms; examples of preferred cycloalkyls are cyclopropyl, cyclobutyl, cyclopentyl, cyclohexyl and cycloheptyl.

Cycloalkyloxy is a monocyclic cycloalkyloxy group having generally 3 to 7, preferably 5 or 6, carbon atoms; examples of preferred cycloalkyloxys are cyclopropyloxy, cyclobutyloxy, cyclopentyloxy and cyclohexyloxy.

Cycloalkylamino is a monocyclic cycloalkylamino group having generally 3 to 7, preferably 3 or 4, carbon atoms; examples of preferred cycloalkylaminos are cyclopropylamino, cyclobutylamino, cyclopentylamino and cyclohexylamino.

Heterocyclyl is a monocyclic or bicyclic, heterocyclic radical having 4 to 7 ring atoms and up to 3, preferably up to 2, heteroatoms and/or hetero groups from the group consisting of N, O, S, SO, SO₂, where one nitrogen atom may also form an N-oxide. The heterocyclyl radicals may be saturated or partially unsaturated. Preference is given to 5- or 6-membered monocyclic saturated heterocyclyl radicals having up to two heteroatoms from the group consisting of O, N and S, by way of example and with preference oxetanyl, azetidinyl, pyrrolidin-2-yl, pyrrolidin-3-yl, pyrrolinyl, tetrahydrofuranyl, tetrahydrothienyl, pyranyl, piperidin-1-yl, piperidin-2-yl, piperidin-3-yl, piperidin-4-yl, 1,2,5,6-tetrahydropyridin-3-yl, 1,2,5,6-tetrahydropyridin-4-yl, thiopyranyl, morpholin-1-yl, morpholin-2-yl, morpholin-3-yl, piperazin-1-yl, piperazin-2-yl, thiomorpholin-2-yl, thiomorpholin-3-yl, thiomorpholin-4-yl, 1-oxidothiomorpholin-4-yl, 1,1-dioxidothiomorpholin-4-yl.

Heterocyclylamino is a monocyclic or bicyclic, heterocyclic heterocyclylamino radical having 4 to 7 ring atoms and up to 3, preferably up to 2, heteroatoms and/or hetero groups from the group consisting of N, O, S, SO, SO₂, where one nitrogen atom may also form an N-oxide. The heterocyclyl radicals may be saturated or partially unsaturated. Preference is given to 5- or 6-membered, monocyclic saturated heterocyclyl radicals having up to two heteroatoms from the group consisting of O, N and S, for example and with preference oxetanylamino, azetidinylamino, pyrrolidin-2-yl-amino, pyrrolidin-3-yl-amino, tetrahydrofuranylamino, tetrahydrothienylamino, pyranylamino, piperidin-2-yl-amino, piperidin-3-yl-amino, piperidin-4-yl-amino, 1,2,5,6-tetra-hydropyridin-3-yl-amino, 1,2,5,6-tetrahydropyridin-4-yl-amino, thiopyranylamino, morpholin-2-yl-amino, morpholin-3-yl-amino, piperazin-2-yl-amino, thiomorpholin-2-yl-amino, thiomorpholin-3-yl-amino.

Heteroaryl is an aromatic monocyclic radical having generally 5 or 6 ring atoms and up to 4 heteroatoms from the group consisting of S, O and N, where one nitrogen atom may also form an N-oxide, by way of example and with preference thienyl, furyl, pyrrolyl, thiazolyl, oxazolyl, isoxazolyl, oxadiazolyl, pyrazolyl, imidazolyl, triazolyl, pyridyl, pyrimidyl, pyridazinyl, pyrazinyl.

Heteroarylamino is an aromatic monocyclic heteroarylamino radical having generally 5 or 6 ring atoms and up to 4 heteroatoms from the group consisting of S, O and N, where one nitrogen atom may also form an N-oxide, by way of example and with preference thienylamino, furylamino, pyrrolylamino, thiazolylamino, oxazolylamino, isoxazolylamino, oxadiazolylamino, pyrazolylamino, imidazolylamino, pyridylamino, pyrimidylamino, pyridazinylamino, pyrazinylamino.

Halogen is fluorine, chlorine, bromine and iodine, preferably fluorine and chlorine.

In the formula of the group which may be A, the end point of the line with a # or a * alongside is not a carbon atom or a CH₂ group, but is part of the bond to the atom to which A is bonded.

Preference is given to compounds of the formula (I) in which
A is an oxygen atom, a sulphur atom, —NR⁴—, —S(O═)— or —S(═O)₂—,
   where
   R⁴ is hydrogen or C₁-C₃-alkyl, or
   R² and R⁴ together with the nitrogen atom to which they are bonded form a 5- or 6-membered heterocycle,
      in which the heterocycle may be substituted by 1 to 3 substituents selected independently from the group consisting of halogen, cyano, hydroxyl, amino, C₁-C₄-alkyl, C₁-C₄-alkoxy and C₁-C₄-alkylamino, or
A is a group of the formula

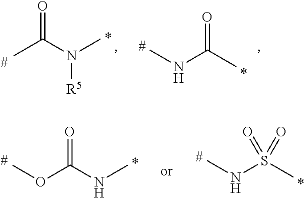

where
# is the attachment site to the piperidine ring,
* is the attachment site of R², and
R⁵ is hydrogen or C₁-C₃-alkyl,
R¹ is phenyl,
   where phenyl is substituted by 1 to 3 substituents selected independently from the group consisting of trifluoromethyl, trifluoromethoxy, C₁-C₄-alkyl, C₁-C₄-alkoxy and C₁-C₄-alkoxycarbonyl,
R² is C₁-C₆-alkyl, C₃-C₆-cycloalkyl, 4- to 6-membered heterocyclyl, phenyl or 5- or 6-membered heteroaryl,
   where cycloalkyl, heterocyclyl, phenyl and heteroaryl may be substituted by 1 to 3 substituents selected independently from the group consisting of halogen, cyano, hydroxyl, amino, trifluoromethyl, difluoromethoxy, trifluoromethoxy, methyl, ethyl, methoxy, ethoxy and phenyl,
      in which phenyl may be substituted by 1 to 3 substituents selected independently from the group consisting of halogen and trifluoromethyl, and
   where C₁-C₆-alkyl may be substituted by one substituent selected from the group consisting of methoxy, ethoxy, methylsulphonyl and phenyl,
      in which phenyl may be substituted by 1 to 3 substituents selected independently from the group consisting of halogen, cyano, trifluoromethyl, trifluoromethoxy, methyl, ethyl, methoxy and ethoxy,
R³ is C₃-C₇-cycloalkyl, 4- to 7-membered heterocyclyl, phenyl, 5- or 6-membered heteroaryl, C₃-C₇-cycloalkyloxy, C₃-C₇-cycloalkylamino, 4- to 7-membered heterocyclylamino, phenylamino or 5- or 6-membered heteroarylamino,
   where cycloalkyl, heterocyclyl, phenyl, heteroaryl, cycloalkyloxy, cycloalkylamino, heterocyclylamino, phenylamino and heteroarylamino may be substituted by 1 to 3 substituents selected independently from the group consisting of halogen, cyano, oxo, hydroxyl, amino, trifluoromethyl, difluoromethoxy, trifluoromethoxy, hydroxycarbonyl, aminocarbonyl, methyl, ethyl, methoxy, ethoxy, dimethylamino, methoxycarbonyl, ethoxycarbonyl, dimethylaminocarbonyl and cyclopropyl,
      in which methyl and ethyl may be substituted by one hydroxyl substituent,
         and their salts, their solvates and the solvates of their salts.

Preference is also given to compounds of the formula (I) in which
A is an oxygen atom, a sulphur atom, —NR⁴—, —S(═O)— or —S(═O)₂—,
   where
   R⁴ is hydrogen, methyl or ethyl, or
   R² and R⁴ together with the nitrogen atom to which they are bonded form a 3-hydroxypyrrolidin-1-yl, or
A is a group of the formula

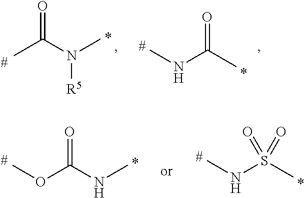

where
# is the attachment site to the piperidine ring,
* is the attachment site of R², and
R⁵ is hydrogen or C₁-C₃-alkyl,
R¹ is phenyl,
   where phenyl is substituted by 1 to 2 substituents selected independently from the group consisting of trifluoromethyl, trifluoromethoxy, methyl, ethyl and methoxy,
R² is methyl, ethyl, propyl, isopropyl, 2-methylprop-1-yl, tert-butyl, cyclopropyl, cyclobutyl, cyclopentyl, cyclohexyl, tetrahydro-2H-pyran-4-yl or phenyl,
   where phenyl may be substituted by 1 to 2 substituents selected independently from the group consisting of halogen, cyano, methyl, ethyl, methoxy and ethoxy, and
   where methyl and ethyl may be substituted by one substituent selected from the group consisting of methoxy, methylsulphonyl and phenyl,
R³ is morpholin-4-yl, 1,1-dioxidothiomorpholin-4-yl, 3-hydroxyazetidin-1-yl, 3-hydroxypyrrolidin-1-yl, 4-cyanopiperidin-1-yl or 4-hydroxypiperidin-1-yl,
   and their salts, their solvates and the solvates of their salts.

Preference is also given to compounds of the formula (I) in which
A is an oxygen atom, a sulphur atom, —NR⁴—, —S(═O)— or —S(═O)₂—,
   where
   R⁴ is hydrogen, methyl or ethyl, or
   R² and R⁴ together with the nitrogen atom to which they are bonded form a 3-hydroxypyrrolidin-1-yl, or
A is a group of the formula

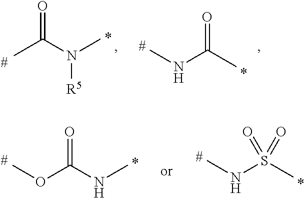

where
# is the attachment site to the piperidine ring,
* is the attachment site of R², and
R⁵ is hydrogen or C₁-C₃-alkyl,
R¹ is phenyl,
   where phenyl is substituted by trifluoromethyl,
R² is methyl, ethyl, propyl, isopropyl, 2-methylprop-1-yl, tert-butyl, cyclopropyl, cyclobutyl, cyclopentyl, cyclohexyl, tetrahydro-2H-pyran-4-yl or phenyl,
   where phenyl may be substituted by 1 to 2 substituents selected independently from the group consisting of halogen, methyl and methoxy, and
   where methyl and ethyl may be substituted by one substituent selected from the group consisting of methoxy, methylsulphonyl and phenyl,
R³ is morpholin-4-yl,
   and their salts, their solvates and the solvates of their salts.

Preference is also given to compounds of the formula (I) in which
A is an oxygen atom, a sulphur atom, —NR⁴—, —S(═O)— or —S(═O)₂—,
R¹ is phenyl,
   where phenyl is substituted by 1 to 2 substituents selected independently from the group consisting of trifluoromethyl, trifluoromethoxy, methyl, ethyl and methoxy,
R² is methyl, ethyl, propyl, isopropyl, 2-methylprop-1-yl, tert-butyl, cyclopropyl, cyclobutyl, cyclopentyl, cyclohexyl, tetrahydro-2H-pyran-4-yl or phenyl,
   where phenyl may be substituted by 1 to 2 substituents selected independently from the group consisting of halogen, cyano, methyl, ethyl, methoxy and ethoxy, and
   where methyl and ethyl may be substituted by one substituent selected from the group consisting of methoxy, methylsulphonyl and phenyl,
R³ is morpholin-4-yl, 1,1-dioxidothiomorpholin-4-yl, 3-hydroxyazetidiny-1-yl, 3-hydroxypyrrolidin-1-yl, 4-cyanopiperidin-1-yl or 4-hydroxypiperidin-1-yl,
   and their salts, their solvates and the solvates of this salts.

Preference is also given to compounds of the formula (I) in which
A is an oxygen atom, a sulphur atom, —NR⁴—, —S(═O)— or —S(═O)₂—,
R¹ is phenyl,
   where phenyl is substituted by 1 to 2 substituents selected independently from the group consisting of trifluoromethyl, trifluoromethoxy, methyl, ethyl and methoxy,
R² is phenyl,
   where phenyl may be substituted by 1 to 2 substituents selected independently from the group consisting of halogen, methyl and methoxy,
R³ is morpholin-4-yl, 1,1-dioxidothiomorpholin-4-yl, 3-hydroxyazetidiny-1-yl, 3-hydroxypyrrolidin-1-yl, 4-cyanopiperidin-1-yl or 4-hydroxypiperidin-1-yl,
   and their salts, their solvates and the solvates of their salts.

Preference is also given to compounds of the formula (I) in which
A is a group of the formula

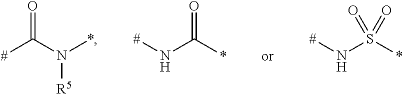

where
# is the attachment site to the piperidine ring,
* is the attachment site of R², and
R⁵ is hydrogen, methyl or ethyl,
R¹ is phenyl,
   where phenyl is substituted by 1 to 2 substituents selected independently from the group consisting of trifluoromethyl, trifluoromethoxy, methyl, ethyl and methoxy,
R² is methyl, ethyl, propyl, isopropyl, 2-methylprop-1-yl, tert-butyl, cyclopropyl, cyclobutyl, cyclopentyl, cyclohexyl, tetrahydro-2h-pyran-4-yl or phenyl,
   where phenyl may be substituted by 1 to 2 substituents selected independently from the group consisting of halogen, cyano, methyl, ethyl, methoxy and ethoxy, and
   where methyl and ethyl may be substituted by one substituent selected from the group consisting of methoxy, methylsulphonyl and phenyl,
R³ is morpholin-4-yl, 1,1-dioxidothiomorpholin-4-yl, 3-hydroxyazetidin-1-yl, 3-hydroxypyrrolidin-1-yl, 4-cyanopiperidin-1-yl or 4-hydroxypiperidin-1-yl,
   and their salts, their solvates and the solvates of their salts.

Preference is also given to compounds of the formula (I) in which
A is a group of the formula

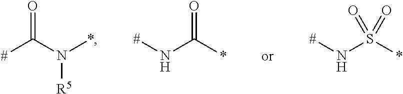

where
# is the attachment site to the piperidine ring,
* is the attachment site to R², and
R⁵ is hydrogen, methyl or ethyl,
R¹ is phenyl,
   where phenyl is substituted by 1 to 2 substituents selected independently from the group consisting of trifluoromethyl, trifluoromethoxy, methyl, ethyl and methoxy,
R² is methyl, ethyl, isopropyl, tert-butyl, cyclopropyl, cyclobutyl, cyclopentyl or cyclohexyl,
   where methyl and ethyl may be substituted by one substituent selected from the group consisting of methoxy and methylsulphonyl,
R³ is morpholin-4-yl, 1,1-dioxidothiomorpholin-4-yl, 3-hydroxyazetidiny-1-yl, 3-hydroxypyrrolidin-1-yl, 4-cyanopiperidin-1-yl or 4-hydroxypiperidin-1-yl,
   and their salts, their solvates and the solvates of their salts.

Preference is also given to compounds of the formula (I) in which the —R¹ and —CH₂-A-R² substituents are in cis-positions to one another.

Preference is also given to compounds of the formula (I) in which the carbon atom to which R¹ is bonded has S configuration and the carbon atom to which —CH₂-A-R² is bonded like has S configuration.

Preference is also given to compounds of the formula (I) in which
A is an oxygen atom, a sulphur atom, —NR⁴—, —S(═O)— or —S(═O)₂—,
   where
   R⁴ is hydrogen, methyl or ethyl, or
   R² and R⁴ together with the nitrogen atom to which they are bonded form a 3-hydroxypyrrolidin-1-yl.

Preference is also given to compounds of the formula (I) in which
A is an oxygen atom, a sulphur atom, —S(═O)— or —S(═O)₂—.

Preference is also given to compounds of the formula (I) in which
A is an oxygen atom or a sulphur atom.

Preference is also given to compounds of the formula (I) in which
A is —S(═O)— or —S(═O)₂—.

Preference is also given to compounds of the formula (I) in which
A is S(═O)—.

Preference is also given to compounds of the formula (I) in which
A is —S(═O)₂—.

Preference is also given to compounds of the formula (I) in which
A is a group of the formula

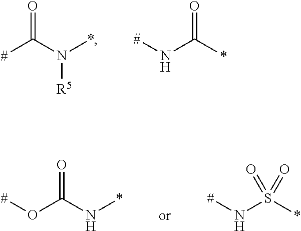

where
# is the attachment site to the piperidine ring,
* is the attachment site to R², and
R⁵ is hydrogen, methyl or ethyl.

Preference is also given to compounds of the formula (I) in which
A is a group of the formula

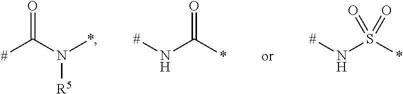

where
# is the attachment site to the piperidine ring,
* is the attachment site to R², and
R⁵ is hydrogen, methyl or ethyl.

Preference is also given to compounds of the formula (I) in which
A is a group of the formula

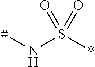

where
# is the attachment site to the piperidine ring,
* is the attachment site to R².

Preference is also given to compounds of the formula (I) in which R¹ is phenyl, where phenyl is substituted by one substituent in the para position to the site of attachment to the piperidine ring, selected from the group consisting of trifluoromethyl, trifluoromethoxy and ethyl.

Preference is also given to compounds of the formula (I) in which R¹ is phenyl, where phenyl is substituted by one trifluoromethyl substituent in the para position to the site of attachment to the piperidine ring.

Preference is also given to compounds of the formula (I) in which
R² is methyl, ethyl, isopropyl, tert-butyl, cyclopropyl, cyclobutyl, cyclopentyl or cyclohexyl,
   where methyl and ethyl may be substituted by one substituent selected from the group consisting of methoxy and methylsulphonyl.

Preference is also given to compounds of the formula (I) in which
R² is phenyl,
   where phenyl may be substituted by 1 to 2 substituents selected independently from the group consisting of halogen, methyl and methoxy.

Preference is also given to compounds of the formula (I) in which R³ is morpholin-4-yl, 1,1-dioxidothiomorpholin-4-yl, 3-hydroxyazetidin-1-yl, 3-hydroxypyrrolidin-1-yl, 4-cyanopiperidin-1-yl or 4-hydroxypiperidin-1-yl.

Preference is also given to compounds of the formula (I) in which R³ is morpholin-4-yl.

The individual radical definitions specified in the respective combinations or preferred combinations of radicals are, independently of the respective combinations of the radicals specified, also replaced as desired by radical definitions of other combinations.

Very particular preference is given to combinations of two or more of the preferred ranges mentioned above.

The invention further provides a process for preparing the compounds of the formula (I), or their salts, their solvates or the solvates of their salts, where either

[A] compounds of the formula

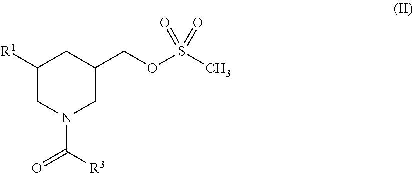

in which
R¹ and R³ are each as defined above
are reacted with compounds of the formula

H-A-R² (III)

in which
R² is as defined above
A is an oxygen atom, a sulphur atom or —NR⁴—, and
R⁴ is as defined above
to give compounds of the formula

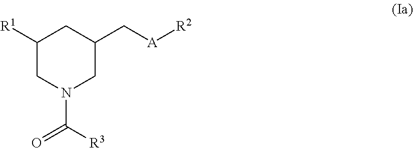

in which
A is an oxygen atom, a sulphur atom or —NR⁴—, and
R¹, R², R³ and R⁴ are each as defined above, or
[B] compounds of the formula (I) in which A is sulphur atom and R′, R² and R³ are each as defined above are reacted with an oxidizing agent
to give compounds of the formula

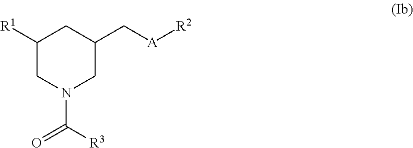

in which

### A is —S(═O)— or —S(═O)₂—, and

R¹, R² and R³ are each as defined above, or
[C] compounds of the formula

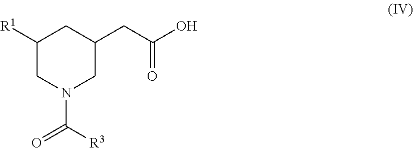

in which
R¹ and R³ are each as defined above
are reacted with compounds of the formula

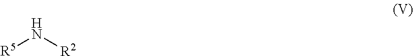

in which
R² and R⁵ are each as defined above
to give compounds of the formula

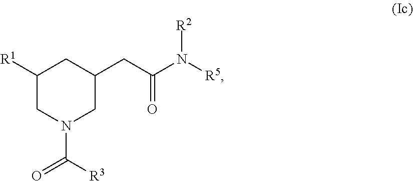

in which
R¹, R², R³ and R⁵ are each as defined above, or
[D] compounds of the formula

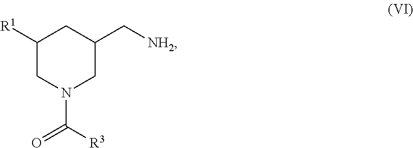

in which
R¹ and R³ are each as defined above
are reacted with compounds of the formula

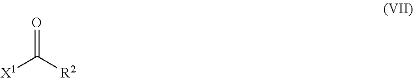

in which
R² is as defined above and
X¹ is halogen, preferably bromine or chlorine, or hydroxyl
to give compounds of the formula

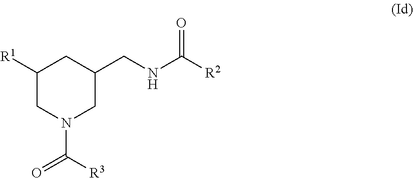

in which
R¹, R² and R³ are each as defined above, or
[E] compounds of the formula (VI) are reacted with compounds of the formula

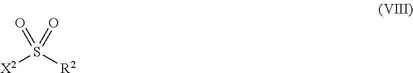

in which

R² is as defined above and

X² is halogen, preferably bromine or chlorine,
to give compounds of the formula

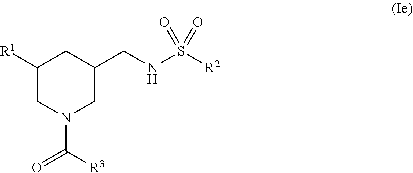

in which
R¹, R² and R³ are each as defined above, or
[F] compounds of the formula

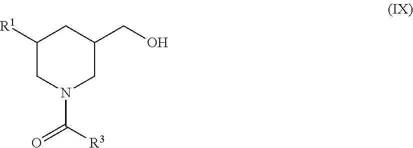

in which
R¹ and R³ are each as defined above
are reacted with compounds of the formula

R²—N— —O (X)

in which
R² is as defined above,
to give compounds of the formula

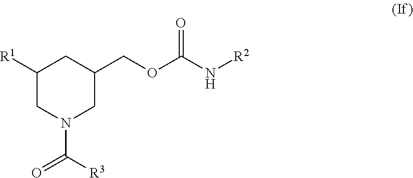

in which
R¹, R² and R³ are as defined above.

The compounds of the formulae (Ia), (Ib), (Ic), (Id), (Ie) and (If) are each a subject of the compounds of the formula (I).

The reaction according to process [A] is generally effected in inert solvents, optionally in the presence of a base, optionally in a microwave, optionally in the presence of molecular sieve, preferably in a temperature range from 0° C. to 150° C. at standard pressure.

Inert solvents are, for example, halohydrocarbons such as methylene chloride, trichloromethane or 1,2-dichloroethane, hydrocarbons such as benzene or toluene, ethers such as diethyl ether, dioxane, tetrahydrofuran or 1,2-dimethoxyethane, or other solvents such as dimethylformamide, dimethylacetamide or dimethyl sulphoxide, preference being given to dimethylformamide.

Alternatively, it is possible to use alcohols such as methanol or ethanol as solvents, in which cases the solvent also simultaneously constitutes the reagent.

Bases are, for example, sodium or potassium methoxide, or sodium or potassium ethoxide or potassium tert-butoxide, or amines such as sodium amide, lithium bis(trimethylsilyl)amide or lithium diisopropylamide, or organometallic compounds such as butyllithium or phenyllithium, or alkali metal hydroxides such as sodium or potassium hydroxide, or other bases such as sodium hydride or potassium hydride, preference being given to sodium hydride.

The compounds of the formula (III) are known or can be synthesized by known processes from the appropriate starting compounds.

The reaction according to process [B] is generally effected in inert solvents, preferably in a temperature range from 0° C. to 50° C. at standard pressure.

Inert solvents are, for example, halohydrocarbons such as methylene chloride, trichloromethane or 1,2-dichloroethane, or alcohols such as methanol or ethanol, or other solvents such as acetic acid, or mixtures of the solvents or mixtures of the solvents with water. preference being given to chloride.

Oxidizing agents are, for example, meta-chloroperoxybenzoic acid, oxone, peroxyacetic acid, hydrogen peroxide, hydrogen peroxide-urea complex or potassium permanganate, preference being given to meta-chloroperoxybenzoic acid.

The reaction is according to the process [C] is generally effected in inert solvents, in the presence of a dehydrating reagent, optionally in the presence of a base, preferably in a temperature range of −30° C. to 50° C. at standard pressure.

Inert solvents are, for example, halohydrocarbons such as dichloromethane or trichloromethane, hydrocarbons such as benzene, nitromethane, dioxane, dimethylformamide or acetonitrile. It is equally possible to use mixtures of the solvents. Particular preference is given to dichloromethane or dimethylformamide

Suitable dehydrating reagents in this context are, for example, carbodiimides, for example N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethylpolystyrene (PS-carbodiimide), or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxy-tri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or benzotriazol-1-yloxytris(pyrrolidino)phosphonium hexafluorophosphate (PYBOP), or N-hydroxysuccinimide, or mixtures of these with bases.

Bases are, for example, alkali metal carbonates, for example sodium carbonate or potassium carbonate, or sodium hydrogencarbonate or potassium hydrogencarbonate, or organic bases such as trialkylamines, for example triethylamine, N-methylmorpholine, N-methylpiperidine, 4-dimethylaminopyridine or diisopropylethylamine.

Preferably, the condensation is performed with HATU in the presence of diisopropylethylamine.

The compounds of the formula (V) are known or can be synthesized by known processes from the appropriate starting compounds.

When X¹ is halogen, the reaction according to process [D] is generally effected in inert solvents, optionally in the presence of a base, preferably in a temperature range of −30° C. to 50° C. at standard pressure.

Inert solvents are, for example, tetrahydrofuran, methylene chloride, pyridine, dioxane or dimethylformamide, preference being to methylene chloride.

Bases are, for example, triethylamine, diisopropylethylamine or N-methylmorpholine, preference being given to triethylamine or diisopropylethylamine

When X¹ is hydroxyl, the reaction according to process [D] is generally effected in inert solvents, in the presence of a dehydrating reagent, optionally in the presence of a base, preferably in a temperature range of −30° C. to 50° C. at standard pressure.

Inert solvents are, for example, halohydrocarbons such as dichloromethane or trichloromethane, hydrocarbons such as benzene, nitromethane, dioxane, dimethylformamide or acetonitrile. It is equally possible to use mixtures of the solvents. Particular preference is given to dichloromethane or dimethylformamide.

Suitable dehydrating reagents in this context are, for example, carbodiimides, for example N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethylpolystyrene (PS-carbodiimide), or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxy-tri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or benzotriazol-1-yloxy-tris(pyrrolidino)phosphonium hexafluorophosphate (PYBOP), or N-hydroxysuccinimide, or mixtures of these, with bases.

Bases are, for example, alkali metal carbonates, for example sodium carbonate or potassium carbonate, or sodium hydrogencarbonate or potassium hydrogencarbonate, or organic bases such as trialkylamines, for example triethylamine, N-methylmorpholine, N-methylpiperidine, 4-dimethylaminopyridine or diisopropylethylamine.

Preferably, the condensation is performed with HATU in the presence of diisopropylethylamine or with PYBOP in the presence of diisopropylethylamine.

The compounds of the formula (VII) are known or can be synthesized by known processes from the appropriate starting compounds.

The reaction according to process [E] is generally effected in inert solvents, optionally in the presence of a base, preferably in a temperature range of 0° C. to 50° C. at standard pressure.

Inert solvents are, for example, tetrahydrofuran, methylene chloride, pyridine, dioxane or dimethylformamide, preference being given to methylene chloride.

Bases are, for example, triethylamine, diisopropylethylamine or N-methylmorpholine, preference being given to triethylamine or diisopropylethylamine

The compounds of the formula (VIII) are known or can be synthesized from the appropriate starting compounds by known processes.

The reaction according to process [F] is generally effected in inert solvents, in the presence of an acid, preferably in a temperature range of 0° C. to 50° C. at standard pressure.

Inert solvents are, for example, tetrahydrofuran, methylene chloride, dioxane or dimethylformamide, preference being given to methylene chloride.

Acids are, for example, hydrogen chloride in dioxane, hydrogen bromide in dioxane or concentrated sulphuric acid in dioxane, preference being given to hydrogen chloride in dioxane.

The compounds of the formula (X) are known or can be synthesized by known processes from the appropriate starting compounds.

The compounds of the formula (II) are known or and can be prepared by hydrogenating compounds of the formula

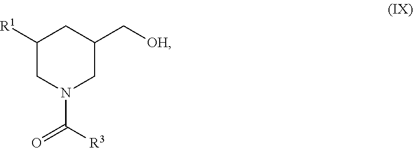

in which
R¹ and R³ are each as defined above,
with methanesulphonyl chloride.

The reaction is generally effected in inert solvents, optionally in the presence of base, preferably within a temperature range from −30° C. to 50° C. at standard pressure.

Inert solvents are, for example, tetrahydrofuran, methylene chloride, pyridine, dioxane, 1,2-dimethoxyethane or dimethylformamide, preference being given to methylene chloride or tetrahydrofuran.

Bases are, for example, triethylamine, diisopropylethylamine, n-methylmorpholine or sodium hydride, preference being given to triethylamine or sodium hydride.

The compounds of the formula (IX) are known or can be prepared by reacting compounds of the formula

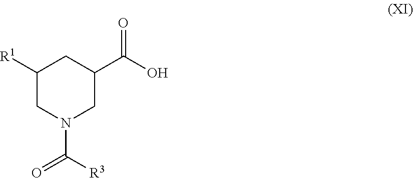

in which
R¹ and R³ are each as defined above
with a reducing agent.

The reaction is effected generally in inert solvents, preferably within a temperature range from −30° C. to 80° C. at standard pressure.

Inert solvents are, for example, ethers such as diethyl ether, tetrahydrofuran, dioxane or 1,2-dimethoxyethane, preference being given to tetrahydrofuran.

Reducing agents are, for example, lithium aluminium hydride, sodium borohydride in conjunction with boron trifluoride-diethyl etherate, lithium borohydride, borane-THF complex, boranedimethyl sulphide complex, preference being given to sodium borohydride in conjunction with boron trifluoride-diethyl etherate.

The compounds of the formula (XI) are known or and can be prepared by reacting compounds of the formula

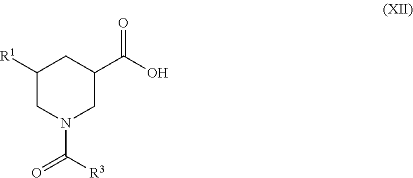

in which
R¹ and R³ are each as defined above and
R⁶ is methyl or ethyl,
with a base.

The reaction is generally effected in inert solvents, in the presence of a base, preferably in a temperature range of room temperature up to reflux of the solvents at standard pressure.

Inert solvents are, for example, halohydrocarbons such as methylene chloride, trichloromethane, tetrachloromethane or 1,2-dichloroethane, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvent with water, preference being given to a mixture of tetrahydrofuran and water.

Bases are, for example, alkali metal hydroxides such as sodium, lithium or potassium hydroxide, or alkali metal carbonates such as caesium carbonate, sodium or potassium carbonate, preference being given to lithium hydroxide.

The compounds of the formula (XII) are known or can be prepared by reacting compounds of the formula

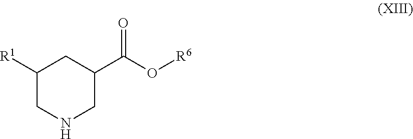

in which
R¹ and R⁶ are each as defined above
with compounds of the formula

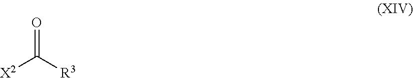

in which
R³ is as defined above and
X² is halogen, preferably bromine or chlorine, or hydroxyl.

When X² is halogen, the reaction is generally effected in inert solvents, optionally in the presence of a base, preferably in a temperature range of −30° C. to 50° C. at standard pressure.

Inert solvents are, for example, tetrahydrofuran, methylene chloride, pyridine, dioxane or dimethylformamide, preference being to tetrahydrofuran.

Bases are, for example, triethylamine, diisopropylethylamine or N-methylmorpholine, preference being given to triethylamine or diisopropylethylamine

When X² is hydroxyl, the reaction is generally effected in inert solvents, in the presence of a dehydrating reagent, optionally in the presence of a base, preferably in a temperature range of −30° C. to 50° C. at standard pressure.

Inert solvents are, for example, halohydrocarbons such as dichloromethane or trichloromethane, hydrocarbons such as benzene, nitromethane, dioxane, dimethylformamide or acetonitrile. It is equally possible to use mixtures of the solvents. Particular preference is given to dichloromethane or dimethylformamide.

Suitable dehydrating reagents in this context are, for example, carbodiimides, for example N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethylpolystyrene (PS-carbodiimide), or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxy-tri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or benzotriazol-1-yloxy-tris(pyrrolidino)phosphonium hexafluorophosphate (PYBOP), or N-hydroxysuccinimide, or mixtures of these, with bases.

Bases are, for example, alkali metal carbonates, for example sodium carbonate or potassium carbonate, or sodium hydrogencarbonate or potassium hydrogencarbonate, or organic bases such as trialkylamines, for example triethylamine, N-methylmorpholine, N-methylpiperidine, 4-dimethylaminopyridine or diisopropylethylamine.

Preferably, the condensation is performed with HATU or with EDC in the presence of HOBt.

The compounds of the formula (XIV) are known or can be synthesized by known processes from the appropriate starting compounds.

The compounds of the formula (XIII) are known or can be prepared by hydrogenating compounds of the formula

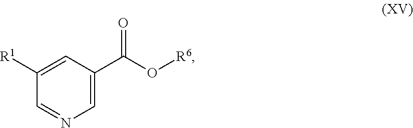

in which
R¹ and R⁶ are each as defined above.

The hydrogenation is generally effected with a reducing agent in inert solvents, optionally with addition of acid such as mineral acids and carboxylic acids, preferably acetic acid, preferably in a temperature range of room temperature up to reflux of the solvents and in a pressure range of standard pressure to 100 bar, preferably at 50-80 bar.

Reducing agents are hydrogen with palladium on activated carbon, with rhodium on activated carbon, with ruthenium on activated carbon or mixed catalysts thereof, or hydrogen with palladium on alumina or with rhodium on alumina, preference being given to hydrogen with palladium on activated carbon or with rhodium on activated carbon.

Inert solvents are, for example, alcohols such as methanol, ethanol, n-propanol, isopropanol, n-butanol or tert-butanol, preference being given to methanol or ethanol.

The compounds of the formula (XV) are known or and can be prepared by reacting compounds of the formula

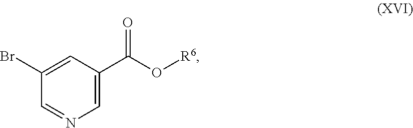

in which
R⁶ is as defined above
with compounds of the formula

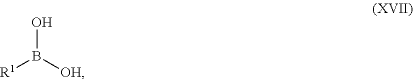

in which
R¹ is as defined above.

The reaction is generally effected in inert solvents, in the presence of a catalyst, optionally in the presence of an additional reagent, preferably in a temperature range of room temperature up to reflux of the solvent at standard pressure.

Inert solvents are, for example, ethers such as dioxane, tetrahydrofuran or 1,2-dimethoxyethane, hydrocarbons such as benzene, xylene or toluene, or other solvents such as nitrobenzene, dimethylformamide, dimethylacetamide, dimethyl sulphoxide or N-methylpyrrolidone; a little water is optionally added to these solvents. Preference is given to toluene with water or to a mixture of 1,2-dimethoxyethane, dimethylformamide and water.

Catalysts are, for example, palladium catalysts customary for Suzuki reaction conditions, preference being given to catalysts such as dichlorobis(triphenylphosphine)palladium, tetrakistriphenylphosphinepalladium(0), palladium(II) acetate or bis(diphenylphosphineferrocenyl)palladium(II) chloride, for example.

Additional reagents are, for example, potassium acetate, caesium, potassium or sodium carbonate, barium hydroxide, potassium tert-butoxide, caesium fluoride, potassium fluoride or potassium phosphate, preference being given to potassium fluoride or sodium carbonate.

The compounds of the formulae (XVI), and (XVII) are known or can be synthesized by known processes from the appropriate starting compounds.

The compound of the formula (IV) are known or can be prepared by reacting compounds of the formula

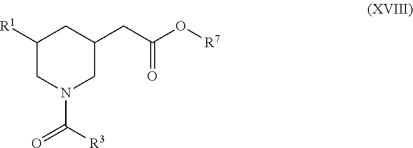

in which
R¹ and R³ are each as defined above, and
R⁷ is methyl or ethyl,
with a base.

The hydrolysis is effected by the reaction conditions specified in the hydrolysis of compounds of the formula (XII).

The compounds of the formula (XVIII) are known or can be prepared by reacting compounds of the formula

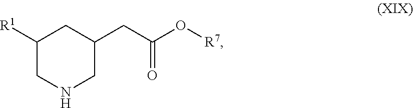

in which
R¹ and R⁷ are each as defined above
with compounds of the formula (XIV).

The reaction is effected by the conditions specified in the reaction of compounds of the formula (XIII) with compounds of the formula (XIV).

The compounds of the formula (XIX) are known or can be prepared by hydrogenating compounds of the formula

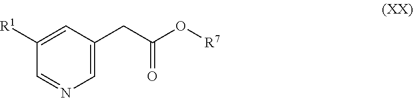

in which
R¹ and R⁷ are each as defined above.

The hydrogenation is effected by the reaction conditions specified in the hydrogenation of compounds of the formula (XV).

The compounds of the formula (XX) are known or can be prepared by reacting compounds of the formula

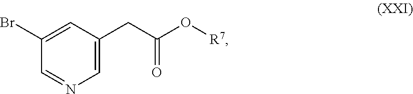

in which
R⁷ is as defined above
with compounds of the formula (XVII).

The reaction is effected by the reaction conditions specified in the reaction of compounds of the formula (XVI) with compounds of the formula (XVII).

The compounds of the formula (XXI) are known or can be synthesized by known processes from the appropriate starting compounds.

The compounds of the formula (VI) are known or can be prepared by reacting compounds of the formula

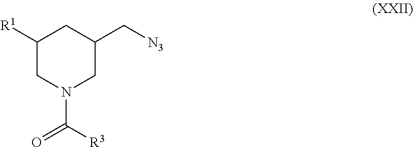

in which
R¹ and R³ are each as defined above
with a reducing agent.

The reaction is effected generally in inert solvents, preferably with a temperature range from room temperature up to the reflux temperature of the solvent and within a pressure range from standard pressure to 100 bar.

Reducing agents are, for example, hydrogen with palladium on activated carbon, with rhodium on activated carbon, with ruthenium on activated carbon or mixed catalysts thereof, or hydrogen with palladium on alumina or with rhodium on alumina, or triphenylphosphine, or cerium(III) chloride heptahydrate with sodium iodide, preference being given to hydrogen with palladium on activated carbon or with rhodium on activated carbon.

Inert solvents are, for example, alcohols such as methanol, ethanol, n-propanol, isopropanol, n-butanol or tert-butanol, preference being given to methanol or ethanol.

The compounds of the formula (X(II) are known or can be prepared by reacting compounds of the formula (II) with sodium azide.

The reaction is generally effected in inert solvents, preferably in a temperature range of room temperature to the reflux temperature of the solvent at standard pressure.

Insert solvents are, for example, halohydrocarbons such as methylene chloride, trichloromethane, tetrachloromethane or 1,2-dichloroethane, or other solvents such as dimethylformamide, dimethylacetamide or acetonitrile, preference being given to dimethylformamide.

In the compounds of the abovementioned processes, free amino groups are optionally protected by protecting groups known to those skilled in the art during the reaction, preference being given to a tert-butoxycarbonyl protecting group. These protecting groups are detached by reactions known to those skilled in the art after the reaction, preference being given to the reaction with trifluoroacetic acid or concentrated hydrochloric acid.

The preparations of the compounds of the formula (I) can be illustrated by the synthesis scheme below.

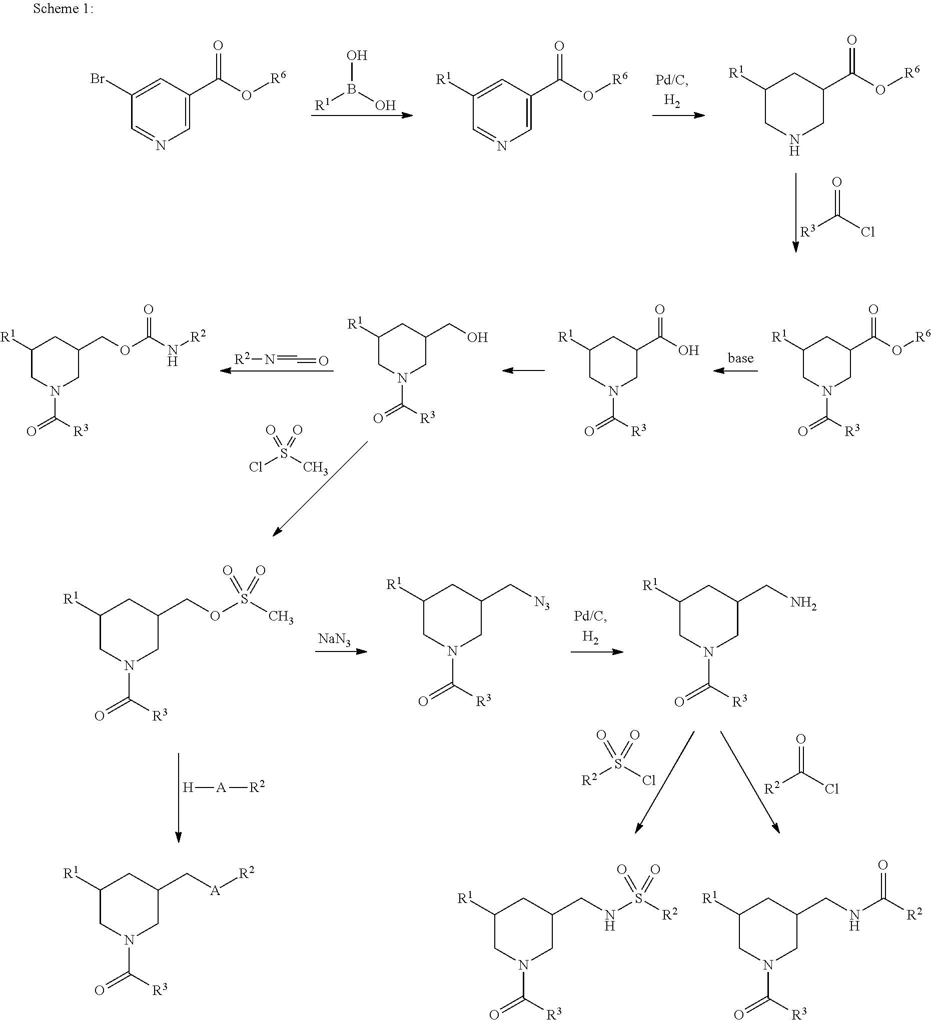

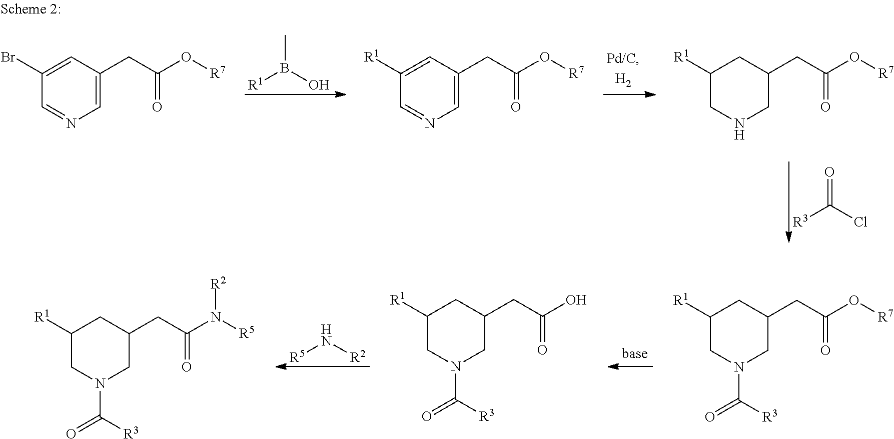

The inventive compounds exhibit an unforeseeable, useful spectrum of pharmacological and pharmacokinetic action. They are selective antagonists of the PAR-1 receptor acting in particular as platelet aggregation inhibitors, as inhibitors of endothelial proliferation and as inhibitors of tumour growth.

They are therefore suitable for use as medicaments for treatment and/or prophylaxis of diseases in man and animals.

The present invention further provides for the use of the inventive compounds for treatment and/or prophylaxis of disorders, preferably of thromboembolic disorders and/or thromboembolic complications.

“Thromboembolic disorders” in the sense of the present invention include in particular disorders such as ST-segment elevation myocardial infarction (STEMI) and non-ST-segment elevation myocardial infarction (non-STEMI), stabile angina pectoris, unstabile angina pectoris, reocclusions and restenoses after coronary interventions such as angioplasty, stent implantations or aortocoronary bypass, peripheral arterial occlusion diseases, pulmonary embolisms, deep venous thromboses and renal vein thromboses, transitory ischaemic attacks and also thrombotic and thromboembolic stroke.

The substances are therefore also suitable for prevention and treatment of cardiogenic thromboembolisms, for example brain ischaemias, stroke and systemic thromboembolisms and ischaemias, in patients with acute, intermittent or persistent cardial arrhythmias, for example atrial fibrillation, and those undergoing cardioversion, and also in patients with heart valve disorders or with intravasal objects, for example artificial heart valves, catheters, intraaortic balloon counterpulsation and pacemaker probes.

Thromboembolic complications are also encountered in connection with microangiopathic haemolytic anaemias, extracorporeal circulation, for example haemodialysis, haemofiltration, ventricular assist devices and artificial hearts, and also heart valve prostheses.

Moreover, the inventive compounds are also used to influence wound healing, for the prophylaxis and/or treatment of atherosclerotic vascular disorders and inflammatory disorders, such as rheumatic disorders of the locomotive system, coronary heart diseases, of heart failure, of hypertension, of inflammatory disorders, for example asthma, COPD, inflammatory pulmonary disorders, glomerulonephritis and inflammatory intestinal disorders, and additionally also for the prophylaxis and/or treatment of Alzheimer's disease, autoimmune disorders, Crohn's disease and ulcerative colitis.

Moreover, the inventive compounds can be used to inhibit tumour growth and metastasization, for microangiopathies, age-related macular degeneration, diabetic retinopathy, diabetic nephropathy and other microvascular disorders, and also for prevention and treatment of thromboembolic complications, for example venous thromboembolisms, for tumour patients, in particular those undergoing major surgical interventions or chemo- or radiotherapy.

The inventive compounds are additionally suitable for treatment of cancer. Cancers include: carcinomas (including breast cancer, hepatocellular carcinomas, lung cancer, colorectal cancer, cancer of the colon and melanomas), lymphomas (for example non-Hodgkin's lymphomas and mycosis fungoides), leukaemias, sarcomas, mesotheliomas, brain cancer (for example gliomas), germinomas (for example testicular cancer and ovarian cancer), choriocarcinomas, renal cancer, cancer of the pancreas, thyroid cancer, head and neck cancer, endometrial cancer, cancer of the cervix, cancer of the bladder, stomach cancer and multiple myeloma.

Moreover, PAR-1 expressed on endothelial cells mediates signals resulting in vascular growth (“angiogenesis”), a process which is vital for enabling tumour growth beyond about 1 mm³ Induction of angiogenesis is also relevant for other disorders, including disorders of the rheumatic type (for example rheumatoid arthritis), pulmonary disorders (for example pulmonary fibrosis, pulmonary hypertension, in particular pulmonary arterial hypertension, disorders characterized by pulmonary occlusion), arteriosclerosis, plaque rupture, diabetic retinopathy and wet macular degeneration.

In addition, the inventive compounds are suitable for the treatment of sepsis. Sepsis (or septicaemia) is a common disorder with high mortality. Initial symptoms of sepsis are typically unspecific (for example fever, reduced general state of health), but there may later be generalized activation of the coagulation system (“disseminated intravascular coagulation” or “consumption coagulopathy”; referred to hereinafter as “DIC”) with the formation of microthrombi in various organs and secondary bleeding complications. Moreover, there may be endothelial damage with increased permeability of the vessels and diffusion of fluid and proteins into the extravasal space. As the disorder worsens, there may be organ dysfunction or organ failure (for example kidney failure, liver failure, respiratory failure, deficits of the central nervous system and heart/circulatory failure) and even multi-organ failure. In principle, this may affect any organ; the most frequently encountered organ dysfunctions and organ failures are those of the lung, the kidney, the cardiovascular system, the coagulation system, the central nervous system, the endocrine glands and the liver. Sepsis may be associated with an “acute respiratory distress syndrome” (referred to hereinafter as ARDS). ARDS may also occur independently of sepsis. “Septic shock” is the occurrence of hypotension which has to be treated and facilitates further organ damage and is associated with a worsening of the prognosis.

Pathogens can be bacteria (gram-negative and gram-positive), fungi, viruses and/or eukaryotes. The site of entry or primary infection may be pneumonia, an infection of the urinary tract or peritonitis, for example. The infection may, but need not necessarily, be associated with bacteriaemia.

Sepsis is defined as the presence of an infection and a “systemic inflammatory response syndrome” (referred to hereinafter as “SIRS”). SIRS occurs during infections, but also during other states such as injuries, burns, shock, operations, ischaemia, pancreatitis, reanimation or tumours. The definition of ACCP/SCCM Consensus Conference Committee of 1992 (Crit. Care Med. 1992, 20, 864-874) describes the symptoms required for the diagnosis of “SIRS” and measurement parameters (including a change in body temperature, increased heart rate, breathing difficulties and changes in the blood picture). The later (2001) SCCM/ESICM/ACCP/ATS/SIS International Sepsis Definitions Conference essentially maintained the criteria, but fine-tuned details (Levy et al., Crit. Care Med. 2003, 31, 1250-1256).

DIC and SIRS may occur during sepsis, but also as a result of operations, tumour disorders, burns or other injuries. In the case of DIC, there is massive activation of the coagulation system at the surface of damaged endothelial cells, the surfaces of foreign bodies or injured extravascular tissue. As a consequence, there is coagulation in small vessels of various organs with hypoxia and subsequent organ dysfunction. A secondary effect is the consumption of coagulation factors (for example factor X, prothrombin, fibrinogen) and platelets, which reduces the coagulability of the blood and may result in heavy bleeding.

In addition, the inventive compounds can also be used for preventing coagulation ex vivo, for example for preserving blood and plasma products, for cleaning/pretreating catheters and other medical aids and instruments, including extracorporeal circulation, for coating synthetic surfaces of medical aids and instruments used in vivo or ex vivo or for platelet-containing biological samples.

The present invention further provides for the use of the inventive compounds for coating medical instruments and implants, for example catheters, prostheses, stents or artificial heart valves. The inventive compounds may be firmly attached to the surface or, for local action, be released over a certain period of time from a carrier coating into the immediate environment.

The present invention further provides for the use of the inventive compounds for treatment and/or prophylaxis of disorders, in particular of the abovementioned disorders.

The present invention further provides for the use of the inventive compounds for production of a medicament for treatment and/or prophylaxis of disorders, in particular of the above-mentioned disorders.

The present invention further provides a method for treatment and/or prophylaxis of disorders, in particular of the abovementioned disorders, using a therapeutically effective amount of an inventive compound.

The present invention further provides medicaments comprising an inventive compound and one or more further active ingredients, in particular for treatment and/or prophylaxis of the abovementioned disorders. Active ingredients suitable for combinations include, by way of example and with preference:

calcium channel blockers, for example amlodipine besilate (for example Norvase), felodipine, diltiazem, verapamil, nifedipine, nicardipine, nisoldipine and bepridil;
iomerizine;
statins, for example atorvastatin, fluvastatin, lovastatin, pitavastatin, pravastatin, rosuvastatin and simvastatin;
cholesterol absorption inhibitors, for example ezetimibe and AZD4121;
cholesteryl ester transfer protein (“CETP”) inhibitors, for example torcetrapib;
low molecular weight heparins, for example dalteparin sodium, ardeparin, certoparin, enoxaparin, parnaparin, tinzaparin, reviparin and nadroparin;
further anticoagulants, for example warfarin, marcumar, fondaparinux;
antiarrhythmics, for example dofetilide, ibutilide, metoprolol, metoprolol tartrate, propranolol, atenolol, ajmaline, disopyramide, prajmaline, procainamide, quinidine, sparteine, aprindine, lidocaine, mexiletine, tocamide, encamide, flecamide, lorcamide, moricizine, propafenone, acebutolol, pindolol, amiodarone, bretylium tosylate, bunaftine, sotalol, adenosine, atropine and digoxin;
alpha-adrenergic agonists, for example doxazosin mesylate, terazoson and prazosin;
beta-adrenergic blockers, for example carvedilol, propranolol, timolol, nadolol, atenolol, metoprolol, bisoprolol, nebivolol, betaxolol, acebutolol and bisoprolol;
aldosterone antagonists, for example eplerenone and spironolactone;
angiotensin-converting enzyme inhibitors (“ACE inhibitors”), for example moexipril, quinapril hydrochloride, ramipril, lisinopril, benazepril hydrochloride, enalapril, captopril, spirapril, perindopril, fosinopril and trandolapril;
angiotensin II receptor blockers (“ARBs”), for example olmesartan-medoxomil, candesartan, valsartan, telmisartan, irbesartan, losartan and eprosartan;
endothelin antagonists, for example tezosentan, bosentan and sitaxsentan-sodium;
inhibitors of neutral endopeptidase, for example candoxatril and ecadotril;
phosphodiesterase inhibitors, for example milrinone, theophylline, vinpocetine, EHNA (erythro-9-(2-hydroxy-3-nonyl)adenine), sildenafil, vardenafil and tadalafil;
fibrinolytics, for example reteplase, alteplase and tenecteplase;
GP IIb/IIIa antagonists, for example integrillin, abciximab and tirofiban;
direct thrombin inhibitors, for example AZD0837, argatroban, bivalirudin and dabigatran;
indirect thrombin inhibitors, for example odiparcil;
direct and indirect factor Xa inhibitors, for example fondaparinux-sodium, apixaban, razaxaban, rivaroxaban (BAY 59-7939), KFA-1982, DX-9065a, AVE3247, otamixaban (XRP0673), AVE6324, SAR377142, idraparinux, SSR126517, DB-772d, DT-831j, YM-150, 813893, LY517717 and DU-1766;
direct and indirect factor Xa/IIa inhibitors, for example enoxaparin-sodium, AVE5026, SSR128428, SSR128429 and BIBT-986 (tanogitran);
lipoprotein-associated phospholipase A2 (“LpPLA2”) modulators;
diuretics, for example chlorthalidone, ethacrynic acid, furosemide, amiloride, chlorothiazide, hydrochlorothiazide, methylclothiazide and benzthiazide;
nitrates, for example isosorbide 5-mononitrate;
thromboxane antagonists, for example seratrodast, picotamide and ramatroban;
platelet aggregation inhibitors, for example clopidogrel, ticlopidine, cilostazol, aspirin, abciximab, limaprost, eptifibatide and CT-50547;
cyclooxygenase inhibitors, for example meloxicam, rofecoxib and celecoxib;
B-type natriuretic peptides, for example nesiritide, ularitide;
NV1FGF modulators, for example XRP0038;
HT1B/5-HT2A antagonists, for example SL65.0472;
guanylate cyclase activators, for example ataciguat (HMR1766) and HMR1069,
e-NOS transcription enhancers, for example AVE9488 and AVE3085;
antiatherogenic substances, for example AGI-1067;
CPU inhibitors, for example AZD9684;
renin inhibitors, for example aliskirin and VNP489;
inhibitors of adenosine diphosphate-induced platelet aggregation, for example clopidogrel, ticlopidine, prasugrel and AZD6140,
NHE-1 inhibitors, for example AVE4454 and AVE4890.

Antibiotic therapy: various antibiotics or antifungal medicament combinations are suitable, either as calculated therapy (before the microbial assessment has been made) or as specific therapy; fluid therapy, for example crystalloid or colloidal fluids; vasopressors, for example norepinephrine, dopamine or vasopressin; inotropic therapy, for example dobutamine; corticosteroids, for example hydrocortisone, or fludrocortisone; recombinant human activated protein C, Xigris; blood products, for example erythrocyte concentrates, platelet concentrates, erythropoietin or fresh frozen plasma; assisted ventilation in sepsis-induced acute lung injury (ALI) or acute respiratory distress syndrome (ARDS), for example permissive hypercapnia, low tidal volumes; sedation: for example diazepam, lorazepam, midazolam or propofol. Opioids: for example fentanyl, hydromorphone, morphine, meperidine or remifentanil. NSAIDs: for example ketorolac, ibuprofen or acetaminophen. Neuromuscular blockade: for example pancuronium; glucose control, for example insulin, glucose; renal replacement therapies, for example continuous veno-venous haemofiltration or intermittent haemodialysis. Low-dose dopamine for renal protection; anticoagulants, for example for thrombosis prophylaxis or for renal replacement therapies, for example unfractionated heparins, low molecular weight heparins, heparinoids, hirudin, bivalirudin or argatroban; bicarbonate therapy; stress ulcer prophylaxis, for example H2 receptor inhibitors, antacids.

Medicaments for proliferative disorders: uracil, chlormethine, cyclophosphamide, ifosfamide, melphalan, chlorambucil, pipobroman, triethylenemelamine, triethylenethiophosphoramine, busulphan, carmustine, lomustine, streptozocin, dacarbazine, methotrexate, 5-fluorouracil, floxuridine, cytarabine, 6-mercaptopurine, 6-thioguanine, fludarabine phosphate, pentostatin, vinblastine, vincristine, vindesine, bleomycin, dactinomycin, daunorubicin, doxorubicin, epirubicin, idarubicin, paclitaxel, mithramycin, deoxycoformycin, mitomycin-C, L-asparaginase, interferons, etoposide, teniposide, 17.alpha.-ethynylestradiol, diethylstilbestrol, testosterone, prednisone, fluoxymesterone, dromostanolone propionate, testolactone, megestrol acetate, tamoxifen, methylprednisolone, methyltestosterone, prednisolone, triamcinolone, chlorotrianisene, hydroxyprogesterone, aminoglutethimide, estranrustine, medroxyprogesterone acetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin (Eloxatie), Iressa (gefmitib, Zd1839), XELODA® (capecitabine), Tarceva® (erlotinib), Azacitidine (5-azacytidine; 5-AzaC), temozolomide (Temodar®), gemcitabine (e.g. GEMZAR® (gemcitabine HCl)), vasostatin or a combination of two or more of the above.

The present invention further provides a method for prevention of blood coagulation in vitro, in particular in banked blood or biological samples containing platelets, which is characterized in that an anticoagulatory amount of the inventive compound is added.

The inventive compounds can act systemically and/or locally. For this purpose, they can be administered in a suitable way, for example, by the oral, parenteral, pulmonary, nasal, sublingual, lingual, buccal, rectal, dermal, transdermal, conjunctival, otic route or as implant or stent.

The inventive compounds can be administered in administration forms suitable for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example, tablets (uncoated or coated tablets, for example having enteric coatings or coatings which are insoluble or dissolve with a delay and control the release of the inventive compound), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration include preparations for injection and infusion in the form of solutions, suspensions, emulsions, lyophilizates or sterile powders.

Oral administration is preferred.

Suitable for the other administration routes are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (e.g. patches), milk, pastes, foams, dusting powders, implants or stents.

The inventive compounds can be converted to the administration forms mentioned. This can be done in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (e.g. antioxidants, for example, ascorbic acid), colours (e.g. inorganic pigments, for example, iron oxides) and masking flavours and/or odours.

The present invention further provides medicaments comprising at least one inventive compound, preferably together with one or more inert, non-toxic, pharmaceutically acceptable excipients, and their use for the purposes mentioned above.

In the case of parenteral administration, it has generally been found to be advantageous to administer amounts of about 5 to 250 mg every 24 hours to achieve effective results. In the case of oral administration the amount is about 5 to 100 mg every 24 hours.

It may nevertheless be necessary where appropriate to deviate from the stated amounts, in particular as a function of the body weight, route of administration, individual response to the active ingredient, nature of the preparation and time or interval over which administration takes place.

The percentages in the tests and examples which follow are, unless stated otherwise, percentages by weight; parts are parts by weight. Solvent ratios, dilution ratios and concentration figures for liquid/liquid solutions are based in each case on volume. “w/v” means “weight/volume”. For example, “10% w/v” means: 100 ml of solution or suspension comprise 10 g of substance.

### A) EXAMPLES

### Abbreviations

approx. approximately
CDI carbonyldiimidazole
d day(s), doublet (in NMR)
TLC thin-layer chromatography
DCI direct chemical ionization (in MS)
dd double doublet (in NMR)
DMAP 4-dimethylaminopyridine
DMF N,N-dimethylformamide
DMSO dimethyl sulphoxide
DPPA diphenyl phosphorazidate
DSC disuccinimidyl carbonate
eq. equivalent(s)
ESI electrospray ionization (in MS)
h hour(s)
HATU O-(7-azabenzotriazol-1-yl)-N,N,N′,N′-tetramethyluronium hexafluorophosphate
HPLC high-pressure, high-performance liquid chromatography
LC-MS liquid chromatography-coupled mass spectroscopy
LDA lithium diisopropylamide
m multiplet (in NMR)
min minute(s)
MS mass spectroscopy
NMR nuclear magnetic resonance spectroscopy
PYBOP benzotriazol-1-yloxytris(pyrrolidino)phosphonium hexafluorophosphate
q quartet (in NMR)
RP reverse phase (in HPLC)
RT room temperature
Rₜ retention time (in HPLC)
s singlet (in NMR)
t triplet (in NMR)
THF tetrahydrofuran
HPLC methods:
   Method 1A: Instrument: HP 1100 with DAD detection; column: Kromasil 100 RP-18, 60 mm×2.1 mm, 3.5 μm; eluent A: 5 ml of perchloric acid (70%)/l of water, eluent B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.
   Method 2A: Instrument: HP 1100 with DAD detection; column: Kromasil 100 RP-18, 60 mm×2.1 mm, 3.5 μm; eluent A: 5 ml of perchloric acid (70%)/l of water, eluent B: acetonitrile; gradient: 0 min 2% B→0.5 min 2% B→4.5 min 90% B→9 min 0% B→9.2 min 2% B→10 min 2% B; flow rate: 0.75 ml/min; column temperature: 30° C.; UV detection: 210 nm.
   Method 3A: Instrument: HP 1100 with DAD detection; column: Kromasil 100 RP-18, 60 mm×2.1 mm, 3.5 μm; eluent A: 5 ml of perchloric acid (70%)/l of water, eluent B: acetonitrile; gradient: 0 min 2% B→0.5 min 2% B→4.5 min 90% B→15 min 90% B→15.2 min 2% B→16 min 2% B; flow rate: 0.75 ml/min; column temperature: 30° C.; UV detection: 210 nm.
   Method 4A: Phase: Kromasil 100, C18, 5 μm, 250 mm×4 mm; Eluent: 50:50 water/acetonitrile; flow rate: 1 ml/min; T: 40° C.; UV: 210 nm.

### LC-MS Methods:

Method 1B: Instrument: Micromass Quattro Premier with Waters HPLC Acquity; column: Thermo Hypersil GOLD 1.9μ 50 mm×1 mm; eluent A: 1 l of water+0.5 ml of 50% formic acid, eluent B: 1 l of acetonitrile+0.5 ml of 50% formic acid; gradient: 0.0 min 90% A→0.1 min 90% A→1.5 min 10% A→2.2 min 10% A; oven: 50° C.; flow rate: 0.33 ml/min; UV detection: 210 nm.
Method 2B: MS instrument type: Waters ZQ; HPLC instrument type: Agilent 1100 Series; UV DAD; column: Thermo Hypersil GOLD 3 μLE 20 mm×4 mm; Eluent A: 1 l of water+0.5 ml of 50% formic acid, eluent B: 1 l of acetonitrile+0.5 ml of 50% formic acid; gradient 0.0 min 100% A→3.0 min 10% A→4.0 min 10% A→4.1 min 100%; oven: 55° C.; flow rate: 2 ml/min; UV detection: 210 nm.
Method 3B: MS instrument type: Micromass ZQ; HPLC instrument type: HP 1100 Series; UV DAD; column: Phenomenex Gemini 3μ 30 mm×3.00 mm; eluent A: 1 l of water+0.5 ml of 50% formic acid, eluent B: 1 l of acetonitrile+0.5 ml of 50% formic acid; gradient 0.0 min 90% A→2.5 min 30% A→3.0 min 5% A→4.5 min 5% A; flow rate: 0.0 min 1 ml/min, 2.5 min/3.0 min/4.5 min. 2 ml/min; oven: 50° C.; UV detection: 210 nm.
Method 4B: MS instrument type: Micromass ZQ; HPLC instrument type: Waters Alliance 2795; column: Phenomenex Synergi 2.5μ MAX-RP 100A Mercury 20 mm×4 mm; eluent A: 1 l of water+0.5 ml of 50% formic acid, eluent B: 1 l of acetonitrile+0.5 ml of 50% formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.
Method 5B: Instrument: Micromass Platform LCZ with HPLC Agilent Series 1100; column: Thermo Hypersil GOLD 3μ 20 mm×4 mm; eluent A: 1 l of water+0.5 ml of 50% formic acid, eluent B: 1 l of acetonitrile+0.5 ml of 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.
Method 6B: Instrument: Micromass Quattro LCZ with HPLC Agilent Series 1100; column: Phenomenex Synergi 2.5μ MAX-RP 100A Mercury 20 mm×4 mm; eluent A: 1 l of water+0.5 ml of 50% formic acid, eluent B: 1 l of acetonitrile+0.5 ml of 50% formic acid; gradient 0.0 min 90% A→0.1 min 90% A→3.0 min 5% A→4.0 min 5% A→4.1 min 90% A; flow rate: 2 ml/min; oven: 50° C.; UV detection: 208-400 nm.
Method 7B: MS instrument type: Waters (Micromass) Quattro Micro; HPLC instrument type: Agilent 1100 Series; column: Thermo Hypersil GOLD 3μ 20 mm×4 mm; eluent A: 1 l of water+0.5 ml of 50% formic acid, eluent B: 1 l of acetonitrile+0.5 ml of 50% formic acid; gradient: 0.0 min 100% A→3.0 min 10% A→4.0 min 10% A→4.01 min 100% A (flow rate 2.5 ml)→5.00 min 100% A; oven: 50° C.; flow rate: 2 ml/min; UV detection: 210 nm.
Method 8B: Instrument: Waters ACQUITY SQD HPLC System; column: Waters Acquity HPLC HSS T3 1.8μ 50 mm×1 mm; eluent A: 1 l of water+0.25 ml 99% formic acid, eluent B: 1 l of acetonitrile+0.25 ml 99 formic acid; gradient: 0.0 min 90% A→1.2 min 5% A→2.0 min 5% A oven: 50° C.; flow rate: 0.40 ml/min; UV detection: 210-400 nm.

### Preparative Separation of Diastereomers:

Method 1C: Phase: Kromasil 100 C18, 5 μm 250 mm×20 mm, eluent: water/acetonitrile 50:50; flow rate: 25 ml/min, temperature: 40° C.; UV detection: 210 nm.
Method 2C: Phase: Sunfire C18, 5 μm OBD 19 mm×150 mm, eluent: water/acetonitrile 62:38; flow rate: 25 ml/min, temperature: 40° C.; UV detection: 210 nm.

### Preparative Separation of Enantiomers:

Method 1D: Phase: Daicel Chiralpak AD-H, 5 μm 250 mm×20 mm; eluent: isohexane/ethanol 50:50; flow rate: 15 ml/min; temperature: 40° C.; UV detection: 220 nm.
Method 2D: Phase: Daicel Chiralpak AD-H, 5 μm 250 mm×20 mm, eluent: isohexane/ethanol 55:45; flow rate: 15 ml/min, temperature: 40° C.; UV detection: 220 nm.
Method 3D: Phase: Daicel Chiralpak AD-H, 5 μm 250 mm×20 mm, eluent: isohexane/ethanol 80:20; flow rate: 15 ml/min, temperature: 40° C.; UV detection: 220 nm.
Method 4D: Phase: Daicel Chiralcel OD-H, 5 μm 250 mm×20 mm, eluent: isohexane/ethanol 90:10; flow rate: 15 ml/min, temperature: 30° C.; UV detection: 220 nm.
Method 5D: Phase: Daicel Chiralpak AD-H, 5 μm 250 mm×20 mm, eluent: tert-butyl methyl ether/methanol 90:10; flow rate: 15 ml/min, temperature: 30° C.; UV detection: 220 nm.

### Analytical Separation of Enantiomers:

Method 1E: Phase: Daicel Chiralpak AD-H, 5 μm 250 mm×4.6 mm; eluent: isohexane/ethanol 50:50; flow rate: 1 ml/min; temperature: 40° C.; UV detection: 220 nm.
Method 2E: Phase: Daicel Chiralpak AS-H, 5 μm 250 mm×4.6 mm; eluent: isohexane/ethanol 50:50; flow rate: 1 ml/min; temperature: 40° C.; UV detection: 220 nm.
Method 3E: Phase: Daicel Chiralpak OD-H, 5 μm 250 mm×4.6 mm; eluent: isohexane/ethanol 85:15; flow rate: 1 ml/min; temperature: 40° C.; UV detection: 220 nm.
Method 4E: Phase: Daicel Chiralpak AD-H, 5 μm 250 mm×4 mm; eluent: tert-butyl methyl ether/methanol: 90:10; flow rate: 1 ml/min; temperature: 25° C.; UV detection: 220 nm.

The microwave reactor used was a “single mode” instrument of the Emrys™ Optimizer type.

### Starting Compounds

### General Method 1A: Suzuki Coupling

A mixture of the appropriate bromopyridine in toluene (1.8 ml/mmol) is admixed under argon at RT with tetrakis(triphenylphosphine)palladium (0.02 eq.), with a solution of the appropriate arylboronic acid (1.2 eq.) in ethanol (0.5 ml/mmol) and with a solution of potassium fluoride (2.0 eq.) in water (0.2 ml/mmol). The reaction mixture is stirred under reflux for several hours until the conversion is substantially complete. After addition of ethyl acetate and phase separation, the organic phase is washed once with water and once with saturated aqueous sodium chloride solution, dried (magnesium sulphate), filtered and concentrated under reduced pressure. The crude product is purified by flash chromatography (silica gel 60, eluent: dichloromethane/methanol mixtures).

### General Method 2A: Hydrogenation of the Pyridine

A solution of the pyridine in ethanol (9 ml/mmol) is admixed under argon with palladium on activated carbon (moistened with approx. 50% water, 0.3 g/mmol), and the mixture is hydrogenated at 60° C. in a 50 bar hydrogen atmosphere overnight. The catalyst is then filtered off through a filter layer and washed repeatedly with ethanol. The combined filtrates are concentrated under reduced pressure.

General Method 3A: Reaction with Carbamoyl Chlorides

A solution of the piperidine in dichloromethane (2.5 ml/mmol) is admixed dropwise under argon at 0° C. with N,N-diisopropylethylamine (1.2 eq.) and the appropriate carbamoyl chloride or carbonyl chloride (1.2 eq.). The reaction mixture is stirred at RT. After addition of water and phase separation, the organic phase is washed three times with water and once with saturated aqueous sodium chloride solution, dried (sodium sulphate), filtered and concentrated under reduced pressure.

### General Method 4A: Hydrolysis

A solution of the appropriate ester in a mixture of tetrahydrofuran/water (3:1, 12.5 ml/mmol) is admixed with lithium hydroxide (2 eq.) at RT. The reaction mixture is stirred at 60° C. and then adjusted to pH 1 with aqueous 1 N hydrochloric acid solution. After addition of water/ethyl acetate, the aqueous phase is extracted three times with ethyl acetate. The combined organic phases are dried (sodium sulphate), filtered and concentrated under reduced pressure.

### General Method 5A: Hydrogenation of the Pyridine Using a Flow Hydrogenation Apparatus

A solution of the pyridine in concentrated acetic acid (about 35 ml/mol) is hydrogenated in a flow hydrogenation apparatus *“H-Cube” from ThalesNano, Budapest Hungary) (conditions: 10% Pd/C catalyst, “controlled” mode, 50 bar, 0.5 ml/min, 85° C.). Removal of the solvent on a rotary evaporator gives the corresponding crude product which is optionally purified by means of preparative HPLC.

### General Method 6A: Methyl Ester Hydrolysis/Epimerization

At RT, potassium tert-butoxide (10 eq.) is added to a solution of the appropriate methyl ester (1.0 eq.) in methanol (35-40 ml/mmol). The mixture is stirred at 60° C. overnight. If the conversion is incomplete, water (1.0 eq.) is added and the mixture is stirred at 60° C. until the conversion is complete. For workup, the methanol is removed under reduced pressure, the residue is admixed with water and the mixture is acidified to pH=1 with 1 N hydrochloric acid. The mixture is extracted with ethyl acetate and the organic phase is dried with magnesium sulphate, filtered and concentrated under reduced pressure.

### Example 1A

### Methyl (5-bromopyridin-3-yl)acetate

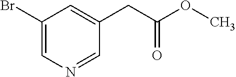

A solution of 3.0 g (13.8 mmol) of 5-bromo-3-pyridylacetic acid in 150 ml of THF was admixed with 3.3 g (30.8 mmol) of O-methyl-N,N′-diisopropylisourea. The reaction mixture was stirred at reflux temperature for 2 hours. For workup, tetrahydrofuran was removed under reduced pressure and the reaction mixture was admixed with ethyl acetate and washed with water and saturated aqueous sodium hydrogencarbonate solution. The organic phase was dried over magnesium sulphate and concentrated under reduced pressure. This gave 4.2 g of crude product in 74% yield (LC-MS), which was converted without further purifying operations.

LC-MS (Method 1B): Rₜ=0.83 min; MS (ESIpos): m/z=231 [M+H]⁺.

### Example 2A

### Methyl {5-[4-(trifluoromethyl)phenyl]pyridin-3-yl}acetate

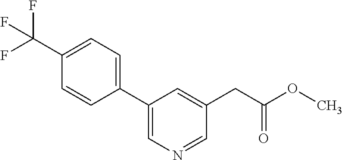

A mixture of 5.5 g (17.9 mmol) of methyl (5-bromopyridin-3-yl)acetate in N,N′-dimethylformamide/1,2-dimethoxyethane (6.3 ml/2.5 ml/mmol) was admixed under argon at room temperature with 206 mg (0.18 mmol) of tetrakis(triphenylphosphine)palladium, 5.11 g (26.9 mmol) of [4-(trifluoromethyl)phenyl]boronic acid, 3.80 g (35.86 mmol) of sodium carbonate and 9 ml of water. The reaction mixture was stirred at 85° C. overnight. For workup, the reaction mixture was concentrated, admixed with ethyl acetate and washed with water. The organic phase was dried over magnesium sulphate and concentrated under reduced pressure. The residue was purified by means of preparative HPLC. Yield: 850 mg (15% of theory)

LC-MS (Method 4B): Rₜ=1.79 min; MS (ESIpos): m/z=296 [M+H]⁺.

### Example 3A

### Methyl {5-[4-(trifluoromethyl)phenyl]piperidin-3-yl}acetate hydroacetate [racemic cis/trans isomer mixture]

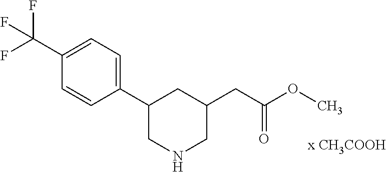

According to General Method 2A, 850 mg (2.88 mmol) of the compound from Example 2A were hydrogenated. Yield: 5.46 g (100% of theory)

LC-MS (Method 4B): Rₜ=1.00 and 1.04 min (cis/trans isomers); MS (ESIpos): m/z=302 [M+H-AcOH]⁺.

### Example 4A

### Methyl {1-(morpholin-4-ylcarbonyl)-5-[4-(trifluoromethyl)phenyl]piperidin-3-yl}acetate [racemic cis/trans isomer mixture]

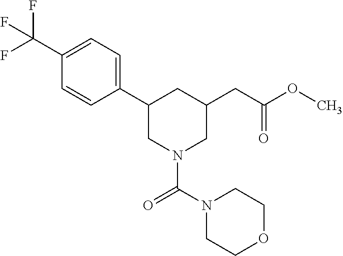

According to General Method 3A, 1.16 g (3.21 mmol) of the compound from Example 3A were reacted with 0.96 g (6.42 mmol) of morpholine-4-carbonyl chloride. This gave 850 mg of crude product in 84% purity (LC-MS), which was converted without further purifying operations.

LC-MS (Method 1B): 11, =1.22 and 1.24 min (cis/trans isomers); MS (ESIpos): m/z=415 [M+H]⁺.

### Example 5A

### {1-(Morpholin-4-ylcarbonyl)-5-[4-(trifluoromethyl)phenyl]piperidin-3-yl}acetic acid [racemic cis/trans isomer mixture]

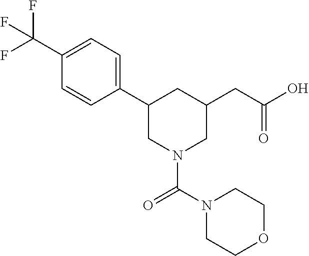

According to General Method 4A, 850 mg (2.1 mmol) of the compound from Example 4A were reacted with 98 mg (4.1 mmol) of lithium hydroxide. This gave 1.0 g of crude product in 88% yield (LC-MS), which was converted without further purifying operations.

LC-MS (Method 3B): Rₜ=2.10 min; MS (ESIpos): m/z=401 [M+H]⁺.

### Example 6A

### 1-(Morpholin-4-ylcarbonyl)-5-[4-(trifluoromethyl)phenyl]-3-hydroxymethylpiperidine [racemic cis/trans isomer mixture]

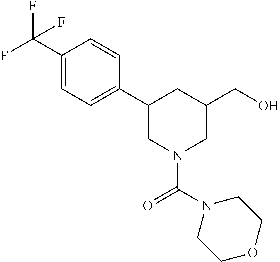

To a suspension, cooled to 0° C., of 1.19 g (31.5 mmol) of sodium borohydride in 7 ml of THF were added dropwise 5.0 g (approx. 10.5 mmol) of 1-(morpholin-4-ylcarbonyl)-5-[4-(trifluoromethyl)phenyl]piperidine-3-carboxylic acid [racemic cis/trans isomer mixture] dissolved in 8 ml of THF. Subsequently, 6 ml (47.2 mmol) of boron trifluoride-diethyl ether complex were added. After the addition had ended, the solution was stirred at room temperature overnight. While cooling with ice, a 1 N hydrochloric acid solution was added to the reaction mixture, which was diluted with water, and the aqueous phase was extracted with dichloromethane. The organic phase was dried over magnesium sulphate, filtered and concentrated under reduced pressure. This gave 3.7 g of crude product, which was converted without further purifying operations.

LC-MS (Method 4B): Rₜ=1.64 min; MS (ESIpos): m/z=373 [M+H]⁺.

### Example 7A

### 1-(Morpholin-4-ylcarbonyl)-5-[4-(trifluoromethyl)phenyl]piperidine-3-methanesulphonate [racemic cis/trans isomer mixture]

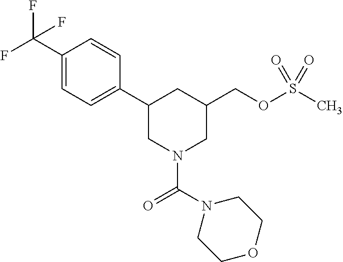

A solution, cooled to 0° C., of 3.59 g (9.65 mmol) of the compound from Example 6A in 68 ml of dichloromethane was admixed with 2.21 g (19.30 mmol) of methanesulphony chloride and 2.7 ml (19.30 mmol) of triethylamine, and the mixture was stirred at room temperature for 16 h. The reaction mixture was admixed with water, and the organic phase was removed and washed with water, dried over magnesium sulphate and concentrated under reduced pressure. The residue was once again dissolved in 120 ml of dichloromethane, cooled to 0° C. and admixed with 13.5 ml (96.48 mmol) of triethylamine and 118 mg (0.96 mmol) of dimethylaminopyridine. Subsequently, 2.21 g (19.30 mmol) of methanesulphonyl chloride was added, and the mixture was stirred at room temperature for 1 h. The reaction mixture was admixed with water, and the organic phase was removed and washed with water, dried over magnesium sulphate and concentrated under reduced pressure. This gave 2.2 g of crude product in 74% purity (LC-MS), which was converted without further purifying operations.

LC-MS (Method 4B): Rₜ=1.88 min; MS (ESIpos): m/z=451 [M+H]⁺.

### Example 8A

### {3-(Azidomethyl)-5-[4-(trifluoromethyl)phenyl]piperidin-1-yl}(morpholin-4-yl)methanone [racemic cis/trans isomer mixture]

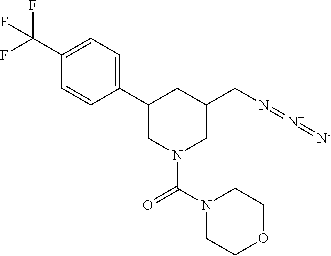

A solution of 3.6 g (approx. 6.47 mmol) of the compound from Example 7A in 220 ml of N,N-dimethylformamide was admixed under argon with 420 mg (6.47 mmol) of sodium azide and stirred at 70° C. overnight. A majority of the solvent was distilled off under reduced pressure and the residue was diluted with ethyl acetate. The mixture was washed repeatedly with saturated aqueous sodium hydrogencarbonate solution, dried over magnesium sulphate, filtered and concentrated to dryness under reduced pressure. This gave 2.55 g of crude product in 78% purity (LC-MS), which was converted without further purifying operations.

LC-MS (Method 3B): Rₜ=2.44 min; MS (ESIpos): m/z=398 [M+H]⁺.

### Example 9A

### {3-(Aminomethyl)-5-[4-(trifluoromethyl)phenyl]piperidin-1-yl}(morpholin-4-yl)methanone [racemic cis/trans isomer mixture]

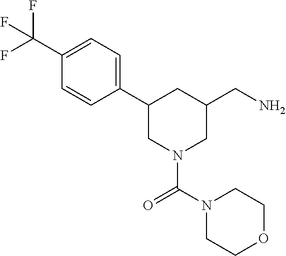

A solution of 2.0 g (approx. 5.03 mmol) of the compound from Example 8A in 70 ml of ethanol, after addition of 536 mg of palladium on activated carbons (50%), was hydrogenated at room temperature and standard pressure for 24 hours. The mixture was filtered through kieselguhr and the residue was washed with ethanol. The filtrate was concentrated to dryness under reduced pressure. This gave 1.79 g of crude product in 63% purity (LC-MS), which was converted without further purifying operations.

LC-MS (Method 3B): Rₜ=1.29 min; MS (ESIpos): m/z=372 [M+H]⁺.

### Example 10A

### Methyl 5-[4-(trifluoromethyl)phenyl]pyridine-3-carboxylate

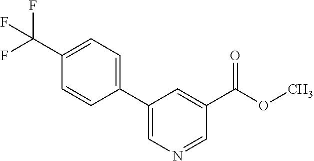

According to General Method 1A, 28 g (132 mmol) of methyl 5-bromonicotinate and 30 g (158 mmol, 1.2 eq.) of 4-trifluoromethylphenylboronic acid were reacted. Yield: 32 g (85% of theory)

LC-MS (Method 4B): Rₜ=2.27 min; MS (ESIpos): m/z=282 [M+H]⁺.

### Example 11A

### Methyl 5-[4-(trifluoromethyl)phenyl]piperidine-3-carboxylate [racemic cis/trans isomer mixture]

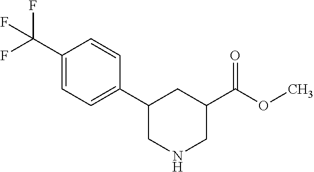

According to General Method 2A, 32 g (112 mmol) of methyl 5-[4-(trifluoromethyl)phenyl]pyridine-3-carboxylate were hydrogenated. Yield: 26 g (82% of theory)

LC-MS (Method 2B): Rₜ=1.35 and 1.41 min (cis/trans isomers); MS (ESIpos): m/z=288 [M+H]⁺.

### Example 12A

### Methyl 1-(morpholin-4-ylcarbonyl)-5-[4-(trifluoromethyl)phenyl]piperidine-3-carboxylate [racemic cis/trans isomer mixture]

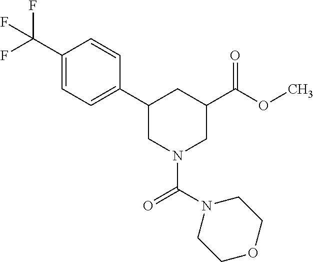

According to General Method 3A, 9.25 g (32.2 mmol) of methyl 5-[4-(trifluoromethyl)phenyl]piperidine-3-carboxylate and 9.63 g (64.7 mmol) of morpholine-4-carbonyl chloride were reacted. This gave 16.3 g of crude product in 76% purity (LC-MS), which was converted without any further purifying operations.

LC-MS (Method 1B): Rₜ=1.19 and 1.22 min (cis/trans isomers); MS (ESIpos): m/z=401 [M+H]⁺.

### Example 13A

### 1-(Morpholin-4-ylcarbonyl)-5-[4-(trifluoromethyl)phenyl]piperidine-3-carboxylic acid [racemic cis isomer]

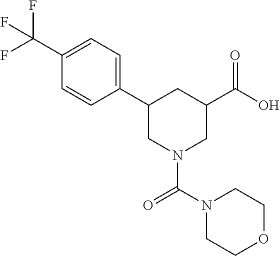

According to General Method 4A, 22.19 g (39.90 mmol) of the compound from Example 12A and 44.78 g (399.0 mmol) of potassium tert-butoxide were reacted. Yield: 18.29 g (100% of theory)

LC-MS (Method 7B): Rₜ=1.95 min; MS (ESIpos): m/z=387 [M+H]⁺.

### Example 14A

### Methyl 1-[(1,1-dioxidothiomorpholin-4-yl)carbonyl]-5-[4-(trifluoromethyl)phenyl]piperidine-3-carboxylate [racemic cis/trans isomer mixture]

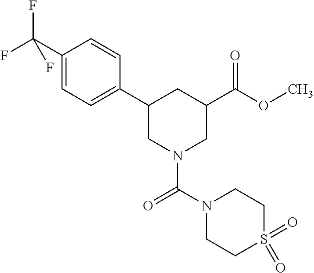

5.19 g (18.1 mmol) of methyl 5-[4-(trifluoromethyl)phenyl]piperidine-3-carboxylate were dissolved in 240 ml of N-methyl-2-pyrrolidone, and admixed at 150° C. with 5.42 g (18.1 mmol) of 4-nitrophenylthiomorpholine-4-carboxylate 1,1-dioxide and 2.75 g (18.1 mmol) of 1,8-diazabicyclo[5.4.0]undec-7-ene. The reaction mixture was stirred for 4.5 h. For workup, the N-methyl-2-pyrrolidone was removed under reduced pressure, and the residue was admixed with ethyl acetate and washed with aqueous 1 N sodium hydroxide solution. The organic phase was dried over magnesium sulphate, filtered and concentrated under reduced pressure. This gave 7.61 g of crude product in 71% purity (LC-MS), which was converted without further purifying operations.

LC-MS (Method 8B): Rₜ=0.94 and 0.96 min; MS (ESIpos): m/z=449 [M+H]⁺.

### Example 15A

### 1-[(1,1-Dioxidothiomorpholin-4-yl)carbonyl]-5-[4-(trifluoromethyl)phenyl]piperidine-3-carboxylic acid [racemic cis isomer]

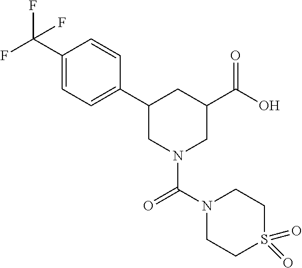

According to General Method 6A, 640 mg (1.09 mmol) of the compound from Example 14A were reacted with 1.23 g (10.1 mmol) of potassium tert-butoxide. Yield: 110 mg (21% of theory)

LC-MS (Method 8B): Rₜ=1.01 min; MS (ESIpos): m/z=435 [M+H]⁺.

### Example 16A

### 1,1-Dioxidothiomorpholin-4-yl) {3-(hydroxymethyl)-5-[4-(trifluoromethyl)phenyl]piperidin-1-yl}-methanone [racemic cis isomer]

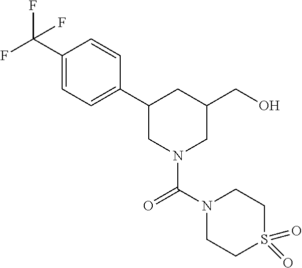

To a suspension, cooled to 0° C., of 19 mg (0.51 mmol) of sodium borohydride in 24 ml of THF were added dropwise 110 mg (0.253 mmol) of the compound from Example 15A dissolved in 12 ml of THF. Subsequently, 0.086 ml (0.68 mmol) of boron trifluoride-diethyl ether complex was added. The reaction mixture was stirred at 0° C. for 0.5 h and then at RT at 2 h. For workup, the reaction mixture was admixed with a 1 N hydrochloric acid solution while cooling with ice and diluted with water, and the aqueous phase was extracted with dichloromethane. The organic phase was dried over magnesium sulphate, filtered and concentrated under reduced pressure. This gave 130 mg of crude product in 76% purity (LC-MS), which was converted without further purifying operations.

LC-MS (Method 8B): Rₜ=0.83 min; MS (ESIpos): m/z=421 [M+H]⁺.

### WORKING EXAMPLES

### General Method 1: Sulphonamide Formation

A solution of the appropriate amine (1.0 eq.) in dichloromethane (21 ml/mmol) is admixed at RT with N,N-diisopropylethylamine (2.5 eq.) and the appropriate sulphonyl chloride (1.5 eq.). The reaction is stirred at room temperature overnight. For workup, dichloromethane is removed under reduced pressure and the residue is purified by means of preparative HPLC.

### General Method 2: Amide Formation 1

A solution of the appropriate amine (1.0 eq.) in dichloromethane (21 ml/mmol) is admixed at room temperature with N,N-diisopropylethylamine (2.5 eq.) and the appropriate carbonyl chloride (1.2 eq.). The reaction is stirred at room temperature overnight. For workup, dichloromethane is removed under reduced pressure and the residue is purified by means of preparative HPLC.

### General Method 3: Amide Formation 2

A solution of 1.0 equivalent of the appropriate carboxylic acid in dimethylformamide is admixed under argon at room temperature with HATU (1.5 eq.) and N,N-diisopropylethylamine (2.5 eq.). After 30 minutes, 1.1 equivalents of the appropriate amine are added. The reaction mixture is stirred at room temperature for 16 h. The reaction mixture is concentrated, and the residue is purified by means of preparative HPLC.

### General Method 4: Amide Formation 3

A solution of 1.0 equivalent of the appropriate carboxylic acid in tetrahydrofuran is admixed under argon at RT with PYBOP (1.5 eq.) and N,N-diisopropylethylamine (7.0 eq.). After 40 minutes, 1.2 equivalents of the appropriate amine are added. The reaction mixture is stirred at RT for 16 h. The reaction mixture is concentrated, and the residue is taken up in ethyl acetate, washed repeatedly with water and a saturated aqueous sodium chloride solution, dried over magnesium sulphate, filtered and concentrated under reduced pressure. The residue is purified by means of preparative HPLC.

### General Method 5: Carbamate Formation 1

A solution of 1.0 equivalent of an alcohol in dichloromethane (2 ml/0.20 mmol) is admixed with a 4 N hydrogen chloride-dioxane solution (0.5 eq.) and the appropriate isocyanate (1.2 eq.). The reaction mixture is stirred at RT for 16 h. The reaction mixture is concentrated and then purified by means of preparative HPLC.

### General Method 6: Carbamate Formation 2

A solution of the appropriate amine (1.0 eq.) in dioxane (15 ml/mmol) is admixed at RT with 4-dimethylaminopyridine (0.1 eq.) and the appropriate isocyanate (1.2 eq.). The reaction mixture is stirred at room temperature overnight. For workup, dioxane is removed under reduced pressure and the residue is purified by means of preparative HPLC.

### General Method 7: Amine

A solution of 1.0 equivalent of a mesylate in N,N-dimethylformamide (27 ml/mmol) is initially charged with molecular sieve and admixed with the appropriate amine under argon. The reaction mixture is stirred at 120° C. for 16 h. The reaction mixture is concentrated, taken up in ethyl acetate, washed repeatedly with a saturated aqueous sodium hydrogencarbonate solution, dried over magnesium sulphate, filtered and concentrated under reduced pressure. The residue is purified by means of preparative HPLC.

### General Method 8: Amines

The solution of a mesylate (1.0 eq.) in N,N′-dimethylformamide (10 ml/mmol) is admixed with the appropriate amine (12.0 eq.) and stirred in a single-mode microwave (Emrys Optimizer) at 150° C. for 0.5 h. The reaction mixture is concentrated, taken up in ethyl acetate, washed repeatedly with a saturated aqueous sodium hydrogencarbonate solution, dried over magnesium sulphate, filtered and concentrated under reduced pressure. The residue is purified by means of preparative HPLC.

### Example 1

### 2,2-Dimethyl-N-({1-(morpholin-4-ylcarbonyl)-5-[4-(trifluoromethyl)phenyl]piperidin-3-yl}methyl)propaneamide [racemic cis isomer]

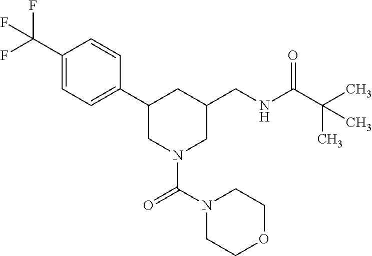

According to General Method 2, 123 mg (approx. 0.21 mmol) of the compound from Example 9A and 30 mg (0.25 mmol) of trimethylacetyl chloride were reacted. Yield: 31 mg (32% of theory)

LC-MS (Method 3B): Rₜ=2.24 min; MS (ESIpos): m/z=456 [M+H]⁺;

¹H NMR (400 MHz, DMSO-d₆): δ=7.68 (d, 2H), 7.59 (t, 1H), 7.51 (d, 2H), 3.63 (br d, 2H), 3.57-3.53 (m, 4H), 3.19-3.08 (m, 5H), 3.04-2.91 (m, 2H), 2.85-2.71 (m, 2H), 1.86 (br d, 1H), 1.77 (m, 1H), 1.34 (q, 1H), 1.09 (s, 9H).

### Example 2

### 3-Chloro-N-({1-(morpholin-4-ylcarbonyl)-5-[4-(trifluoromethyl)phenyl]piperidin-3-yl}methyl)benzenecarboxamide [racemic cis isomer]

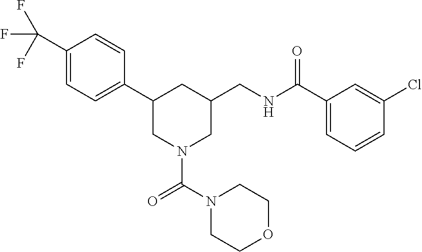

According to General Method 2, 100 mg (approx. 0.10 mmol) of the compound from Example 9A and 29 mg (0.17 mmol) of 3-chlorobenzoyl chloride were converted. Yield: 5 mg (8% of theory)

LC-MS (Method 1B): Rₜ=1.30 min; MS (ESIpos): m/z=510 [M+H]⁺;

¹H NMR (400 MHz, DMSO-d₆): δ=8.71 (t, 1H), 7.89 (s, 1H), 7.81 (d, 1H), 7.68 (d, 2H), 7.61 (br d, 1H), 7.55-7.50 (m, 3H), 3.70 (br d, 1H), 3.63 (d, 1H), 3.51-3.47 (m, 4H), 3.25-3.18 (m, 2H), 3.13-3.05 (m, 5H), 2.91-2.79 (m, 2H), 1.96-1.85 (m, 2H), 1.44 (q, 1H).

### Example 3

### 2-(Methylsulphonyl)-N-({1-(morpholin-4-ylcarbonyl)-5-[4-(trifluoromethyl)phenyl]piperidin-3-yl}methyl)acetamide [racemic cis isomer]

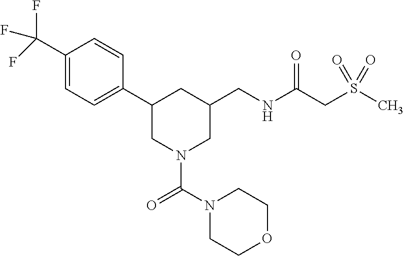

According to General Method 3, 88 mg (approx. 0.15 mmol) of the compound from Example 9A and 19 mg (0.14 mmol) of (methylsulphonyl)acetic acid were reacted. Diastereomer separation of 38 mg of the residue by Method 1C gave 2 mg of Example 3.

LC-MS (Method 1B): Rₜ=1.05 min; MS (ESIpos): m/z=492 [M+H]⁺.

### Example 4

### 3-Fluoro-N-({1-(morpholin-4-ylcarbonyl)-5-[4-(trifluoromethyl)phenyl]piperidin-3-yl}methyl)benzenecarboxamide [racemic cis isomer]

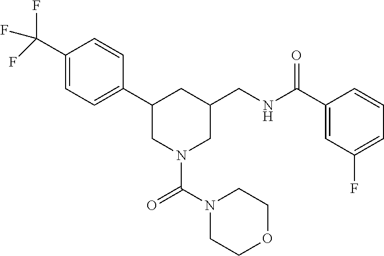

According to General Method 2, 131 mg (approx. 0.22 mmol) of the compound from Example 9A and 42 mg (0.27 mmol) of 3-fluorobenzoyl chloride were reacted. Yield: 11 mg (10% of theory)

LC-MS (Method 1B): Rₜ=1.25 min; MS (ESIpos): m/z=494 [M+H]⁺;

¹H NMR (400 MHz, DMSO-d₆): δ=8.72-8.70 (m, 1H), 7.89 (br s, 1H), 7.81 (d, 1H), 7.68 (d, 2H), 7.61 (br d, 1H), 7.54-7.49 (m, 3H), 3.67 (dd, 2H), 3.56-3.42 (m, 5H), 3.25-3.18 (m, 2H), 3.16-3.04 (m, 4H), 2.91-2.79 (m, 2H), 1.96-1.90 (m, 2H), 1.45 (q, 1H).

### Example 5

### 3-Methoxy-N-({1-(morpholin-4-ylcarbonyl)-5-[4-(trifluoromethyl)phenyl]piperidin-3-yl}methyl)benzenecarboxamide [enantiomerically pure cis isomer]

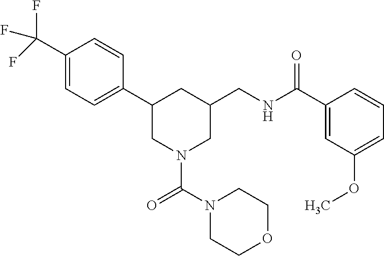

According to General Method 2, 129 mg (approx. 0.22 mmol) of the compound from Example 9A and 45 mg (0.26 mmol) of 3-methoxybenzenecarbonyl chloride were reacted. Enantiomer separation of 47 mg of the racemate by Method 1D gave 9 mg of Example 5.

HPLC (Method 1E): Rₜ=8.35 min, >97.0% ee; LC-MS (Method 3B): Rₜ=2.33 min; MS (ESIpos): m/z=506 [M+H]⁺;

¹H NMR (400 MHz, DMSO-d₆): δ=8.57 (t, 1H), 7.68 (d, 2H), 7.53 (d, 2H), 7.44-7.36 (m, 3H), 7.08 (dd, 1H), 3.80 (s, 3H), 3.70 (br d, 1H), 3.64 (d, 1H), 3.50-3.46 (m, 4H), 3.23-3.18 (m, 2H), 3.14-3.06 (m, 4H), 2.92-2.79 (m, 2H), 2.00-1.90 (m, 2H), 1.93 (q, 1H).

### Example 6

### N-({1-(Morpholin-4-ylcarbonyl)-5-[4-(trifluoromethyl)phenyl]piperidin-3-yl}methyl)methanesulphonamide [enantiomerically pure cis isomer]

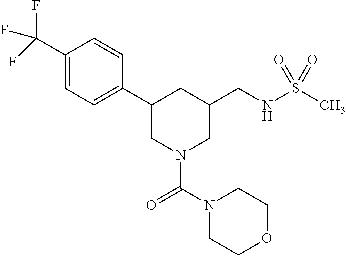

According to General Method 1, 104 mg (approx. 0.18 mmol) of the compound from Example 9A and 24 mg (0.21 mmol) of methanesulphonyl chloride were reacted. Enantiomer separation of the residue by Method 2D gave 8 mg of Example 6 (Enantiomer 1) and 7 mg of Example 7 (Enantiomer 2).

HPLC (Method 1E): Rₜ=10.28 min, >99.0% ee; LC-MS (Method 2B): Rₜ=2.01 min; MS (ESIpos): m/z=450 [M+H]⁺;

¹H NMR (400 MHz, DMSO-d₆): δ=7.68 (d, 2H), 7.52 (d, 2H), 7.13 (t, 1H), 3.77 (br d, 3H), 3.63 (d, 1H), 3.58-3.51 (m, 4H), 3.16-3.10 (m, 4H), 2.90 (s, 3H), 2.93-2.76 (m, 3H), 1.93 (br d, 1H), 1.79 (br s, 1H), 1.37 (q, 1H).

### Example 7

### N-({1-(Morpholin-4-ylcarbonyl)-5-[4-(trifluoromethyl)phenyl]piperidin-3-yl}methyl)methanesulphonamide [enantiomerically pure cis isomer]

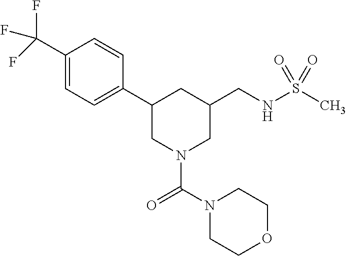

According to General Method 1, 104 mg (approx. 0.18 mmol) of the compound from Example 9A and 24 mg (0.21 mmol) of methanesulphonyl chloride were reacted. Enantiomer separation of the residue by Method 2D gave 8 mg of Example 6 (Enantiomer 1) and 7 mg of Example 7 (Enantiomer 2).

HPLC (Method 1E): Rₜ=12.14 min, >99.0% ee; LC-MS (Method 2B): Rₜ=2.01 min; MS (ESIpos): m/z=450 [M+H]⁺.

### Example 8

### 2-Fluoro-N-({1-(morpholin-4-ylcarbonyl)-5-[4-(trifluoromethyl)phenyl]piperidin-3-yl}methyl)benzenesulphonamide [enantiomerically pure cis isomer]

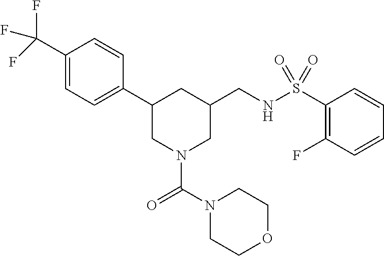

According to General Method 1, 126 mg (approx. 0.21 mmol) of the compound from Example 9A and 49 mg (0.21 mmol) of 2-fluorobenzenesulphonyl chloride were reacted. Enantiomer separation of the residue by Method 3D gave 10 mg of Example 8 (Enantiomer 1) and 10 mg of Example 9 (Enantiomer 2).

HPLC (Method 2E): Rₜ=11.53 min, >98.0% ee; LC-MS (Method 3B): Rₜ=2.42 min; MS (ESIpos): m/z=530 [M+H]⁺;

¹H-NMR (400 MHz, DMSO-d₆): δ=8.07 (br s, 1H), 7.79 (dt, 1H), 7.74-7.67 (m, 3H), 7.47 (d, 2H), 7.44-7.37 (m, 2H), 3.72 (br d, 1H), 3.60 (d, 1H), 3.58-3.53 (m, 4H), 3.17-3.09 (m, 4H), 2.86-2.70 (m, 4H), 2.43 (t, 1H), 1.86 (br d, 1H), 1.74 (br s, 1H), 1.30 (q, 1H).

### Example 9

### 2-Fluoro-N-({1-(morpholin-4-ylcarbonyl)-5-[4-(trifluoromethyl)phenyl]piperidin-3-yl}methyl)benzenesulphonamide [enantiomerically pure cis isomer]

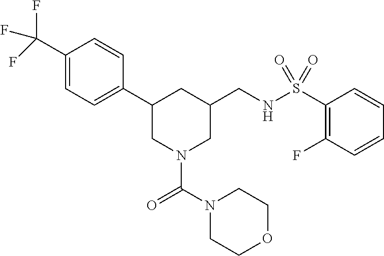

According to General Method 1 126 mg (approx. 0.21 mmol) of the compound from Example 9A and 49 mg (0.21 mmol) of 2-fluorobenzenesulphonyl chloride were reacted. Enantiomer separation of the residue by Method 3D gave 10 mg of Example 8 (Enantiomer 1) and 10 mg of Example 9 (Enantiomer 2).

HPLC (Method 2E): Rₜ=13.21 min, >96.0% ee; LC-MS (Method 1B): Rₜ=1.27 min; MS (ESIpos): m/z=530 [M+H]⁺.

### Example 10

### {1-(Morpholin-4-ylcarbonyl)-5-[4-(trifluoromethoxy)phenyl]piperidin-3-yl}methyl (3-fluorophenyl)carbamate [racemic cis isomer]

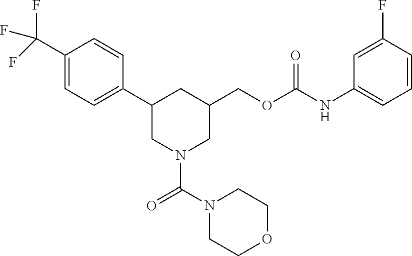

According to General Method 6, 100 mg (0.26 mmol) of the compound from Example 6A and 42 mg (0.31 mmol) of 3-fluorophenyl isocyanate were converted. Yield: 24 mg (17% of theory)

LC-MS (Method 4B): Rₜ=2.35 min; MS (ESIpos): m/z=526 [M+H]⁺;

¹H NMR (400 MHz, DMSO-d₆): δ=9.91 (s, 1H), 7.44-7.28 (m, 6H), 7.22 (d, 1H), 6.81 (dt, 1H), 4.06 (dd, 1H), 4.00 (dd, 1H), 3.78 (br d, 1H), 3.62 (d, 1H), 3.56-3.53 (m, 4H), 3.18-3.10 (m, 4H), 2.90-2.76 (m, 2H), 2.60 (t, 1H), 2.09-1.09 (m, 1H), 1.95 (br d, 1H), 1.45 (q, 1H).

### Example 11

### {1-(Morpholin-4-ylcarbonyl)-5-[4-(trifluoromethoxy)phenyl]piperidin-3-yl}methyl (2-chlorophenyl)carbamate [racemic cis isomer]

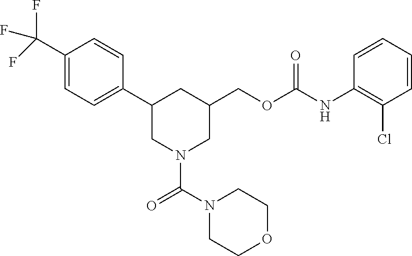

According to General Method 6, 50 mg (0.13 mmol) of the compound from Example 6A and 23 mg (0.15 mmol) of 2-chlorophenyl isocyanate were reacted. Yield: 18 mg (26% of theory)

LC-MS (Method 3B): Rₜ=2.82 min; MS (ESIpos): m/z=542 [M+H]⁺.

### Example 12

### {1-(Morpholin-4-ylcarbonyl)-5-[4-(trifluoromethoxy)phenyl]piperidin-3-yl}methyl (4-methylphenyl)carbamate

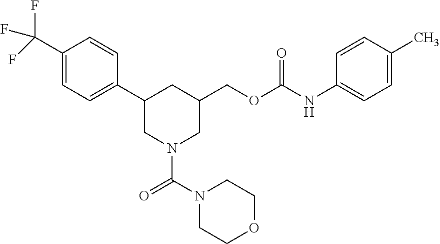

According to General Method 6, 50 mg (0.13 mmol) of the compound from Example 6A and 20 mg (0.15 mmol) of 1-isocyanato-4-methylbenzene were reacted. Yield: 21 mg (32% of theory)

LC-MS (Method 4B): Rₜ=2.35 min; MS (ESIpos): m/z=522 [M+H]⁺.

### Example 13

### N-Benzyl-2-{1-(morpholin-4-ylcarbonyl)-5-[4-(trifluoromethyl)phenyl]piperidin-3-yl}acetamide [enantiomerically pure cis isomer]

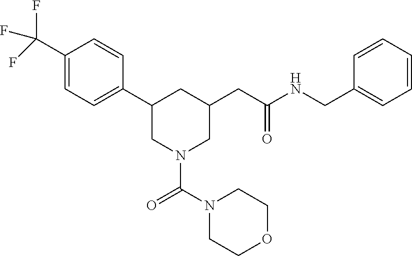

According to General Method 3, 100 mg (approx. 0.23 mmol) of the compound from Example 5A and 28 mg (0.27 mmol) of benzylamines were reacted. Enantiomer separation of the residue by Method 4D gave 17 mg of Example 13.

HPLC (Method 3E): Rₜ=10.04 min, >99.0% ee; LC-MS (Method 1B): Rₜ=1.23 min; MS (ESIpos): m/z=490 [M+H]⁺;

¹H NMR (400 MHz, DMSO-d₆): δ=8.41 (t, 1H), 7.68 (d, 2H), 7.50 (d, 2H), 7.31-7.20 (m, 5H), 4.31-4.21 (m, 2H), 3.68 (d, 1H), 3.65 (d, 1H), 3.55-3.50 (m, 4H), 3.29 (s, 2H), 3.13-3.09 (m, 4H), 2.88 (ft, 1H), 2.76 (t, 1H), 2.14-2.12 (m, 2H), 2.07-2.00 (m, 1H), 1.90 (br d, 1H), 1.38 (q, 1H).

### Example 14

### N,N-Diethyl-2-{1-(morpholin-4-ylcarbonyl)-5-[4-(trifluoromethyl)phenyl]piperidin-3-yl}acetamide [racemic cis isomer]

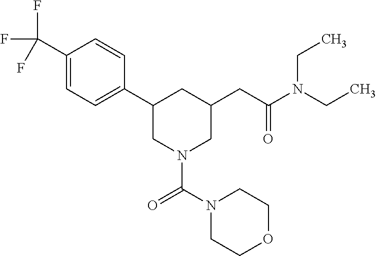

According to General Method 3, 100 mg (approx. 0.23 mmol) of the compound from Example 5A and 20 mg (0.27 mmol) of diethylamine were converted. Diastereomer separation of the residue by Method 2C gave 51 mg of Example 14.

LC-MS (Method 3B): Rₜ=2.50 min; MS (ESIpos): m/z=456 [M+H]⁺;

¹H NMR (400 MHz, DMSO-d₆): δ=7.68 (d, 2H), 7.51 (d, 2H), 3.72-3.65 (m, 2H), 3.60-3.53 (m, 4H), 3.30-3.23 (m, 5H), 3.20-3.10 (m, 4H), 2.95-2.83 (m, 1H), 2.72 (t, 1H), 2.25 (d, 2H), 2.13-2.00 (m, 1H), 1.93 (br d, 1H), 1.43 (q, 1H), 1.09 (t, 3H), 1.00 (t, 3H).

### Example 15

### 2-{1-(Morpholin-4-ylcarbonyl)-5-[4-(trifluoromethyl)phenyl]piperidin-3-yl}-N-phenylacetamide [racemic cis isomer]

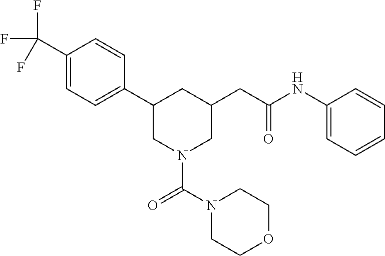

According to General Method 3, 100 mg (approx. 0.23 mmol) of the compound from Example 5A and 25 mg (0.27 mmol) of aniline were reacted. Diastereomer separation of the residue by Method 2C gave 4 mg of Example 15.

LC-MS (Method 1B): Rₜ=1.26 min; MS (ESIpos): m/z=476 [M+H]⁺;

¹H NMR (400 MHz, DMSO-d₆): δ=9.94 (s, 1H), 7.68 (d, 2H), 7.58 (d, 2H), 7.52 (d, 4H), 7.28 (dd, 2H), 7.02 (dd, 1H), 3.75 (br d, 1H), 3.66 (br d, 1H), 3.56-3.50 (m, 4H), 3.16-3.10 (m, 4H), 2.97-2.87 (m, 1H), 2.80 (t, 1H), 2.35-2.29 (m, 2H), 2.17-2.05 (m, 1H), 1.96 (br d, 1H), 1.46 (q, 1H).

### Example 16

### {1-(Morpholin-4-ylcarbonyl)-5-[4-(trifluoromethyl)phenyl]piperidin-3-yl}methyl isobutylcarbamate [racemic cis isomer]

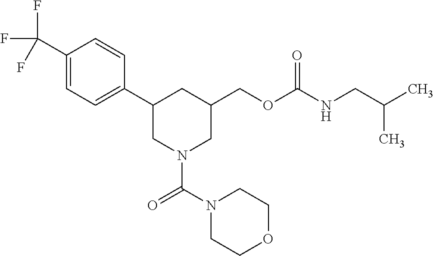

According to General Method 5, 100 mg (0.196 mmol) of the compound from Example 6A and 23 mg (0.235 mmol) of isobutyl isocyanate were reacted. Yield: 7 mg (12% of theory)

HPLC (Method 8B): Rₜ=1.16 min; MS (ESIpos): m/z=472 [M+H]⁺;

¹H NMR (400 MHz, DMSO-d₆): δ=7.69 (d, 2H), 7.52 (d, 2H), 7.19 (t, 1H), 3.91-3.85 (m, 2H), 3.72 (d, 1H), 3.64 (d, 1H), 3.60-3.53 (m, 4H), 3.18-3.10 (m, 4H), 2.94-2.85 (m, 1H), 2.83-2.77 (m, 3H), 1.98-1.84 (m, 2H), 1.70-1.58 (m, 1H), 1.42 (q, 1H), 0.82 (d, 6H).

### Example 17

### {1-(Morpholin-4-ylcarbonyl)-5-[4-(trifluoromethyl)phenyl]piperidin-3-yl}methyl ethyl carbamate [racemic cis isomer]

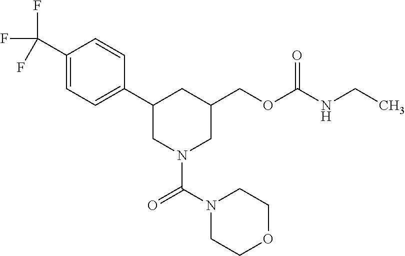

According to General Method 5, 200 mg (0.392 mmol) of the compound from Example 6A and 33 mg (0.470 mmol) of ethyl isocyanate were reacted. Yield: 73 mg (40% of theory)

HPLC (Method 8B): Rₜ=1.06 min; MS (ESIpos): m/z=444 [M+H]⁺;

¹H NMR (400 MHz, DMSO-d₆): δ=7.69 (d, 2H), 7.52 (d, 2H), 7.13 (t, 1H), 3.91 (dd, 1H), 3.86-3.59 (m, 3H), 3.56 (t, 4H), 3.14 (d, 4H), 3.05-2.95 (m, 2H), 2.95-2.73 (m, 2H), 1.91 (br s, 2H) 1.42 (d, 1H) 1.01 (t, 3H).

### Example 18

### {1-(Morpholin-4-ylcarbonyl)-5-[4-(trifluoromethyl)phenyl]piperidin-3-yl}methyl (2-methoxyethyl)carbamate [racemic cis isomer]

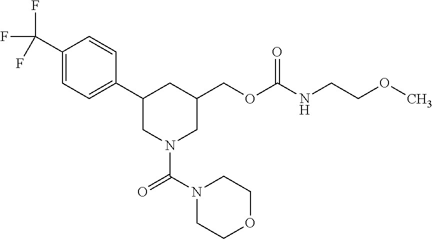

According to General Method 5, 200 mg (0.392 mmol) of the compound from Example 6A and 48 mg (0.470 mmol) of 1-isocyanato-2-methoxyethane were reacted. Yield: 65 mg (35% of theory) HPLC (Method 8B): Rₜ=1.02 min; MS (ESIpos): m/z=474 [M+H]⁺;

¹H NMR (400 MHz, DMSO-d₆): δ=7.69 (d, 2H), 7.52 (d, 2H), 7.20 (t, 1H, 3.95-3.79 (m, 2H), 3.72 (d, 1H), 3.64 (d, 1H), 3.60-3.49 (m, 4H), 3.22 (s, 3H), 3.19-3.06 (m, 6H), 2.94-2.86 (m, 1H), 2.78 (t, 1H), 1.91 (br s, 2H), 1.42 (q, 1H).

### Example 19

### {1-(Morpholin-4-ylcarbonyl)-5-[4-(trifluoromethyl)phenyl]piperidin-3-yl}methyl isopropylcarbamate [racemic cis isomer]

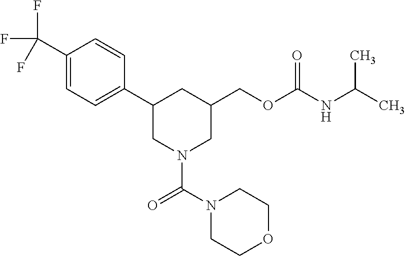

According to General Method 5, 200 mg (0.392 mmol) of the compound from Example 6A and 40 mg (0.470 mmol) of 2-isocyanatopropane were reacted. Yield: 86 mg (48% of theory) HPLC (Method 8B): Rₜ=1.11 min; MS (ESIpos): m/z=458 [M+H]⁺;

¹H NMR (400 MHz, DMSO-d₆): δ=7.69 (d, 2H), 7.51 (s, 2H), 7.05 (d, 1H), 3.94-3.78 (m, 2H), 3.76-3.50 (m, 7H), 3.20-3.18 (m, 4H), 2.95-2.80 (m, 1H), 2.78 (t, 1H), 2.00-1.85 (d, 2H), 1.43 (q, 1H), 1.06 (d, 6H).

### Example 20

### {1-(Morpholin-4-ylcarbonyl)-5-[4-(trifluoromethyl)phenyl]piperidin-3-yl}methyl (3-methoxy-2,2-dimethylpropyl)carbamate [racemic cis isomer]

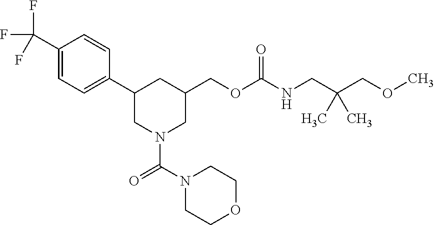

According to General Method 5, 200 mg (0.392 mmol) of the compound from Example 6A and 67 mg (0.470 mmol) of 1-isocyanato-3-methoxy-2,2-dimethylpropane were reacted. Yield: 40 mg (20% of theory)

HPLC (Method 1B): Rₜ=1.32 min; MS (ESIpos): m/z=516 [M+H]⁺;

¹H NMR (400 MHz, DMSO-d₆): δ=7.69 (d, 2H), 7.52 (d, 2H), 7.06 (t, 1H), 3.90 (dm, 2H), 3.74 (d, 1H), 3.64 (d, 1H), 3.59-3.52 (m, 4H), 3.21 (s, 3H), 3.18-3.01 (m., 4H), 3.01 (s, 3H), 2.95-2.85 (m, 3H), 2.79 (t, 1H), 2.01-1.85 (m, 2H), 1.42 (q, 1H), 0.79 (s, 6H).

### Example 21

### {1-(Morpholin-4-ylcarbonyl)-5-[4-(trifluoromethyl)phenyl]piperidin-3-yl}methyl propylcarbamate [racemic cis isomer]

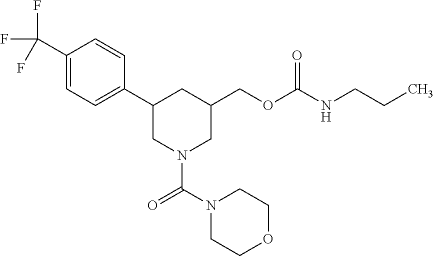

According to General Method 5, 200 mg (0.392 mmol) of the compound from Example 6A and 40 mg (0.470 mmol) of isobutylisocyanate were reacted. Yield: 71 mg (40% of theory) HPLC (Method 8B): Rₜ=1.11 min; MS (ESIpos): m/z=458 [M+H]⁺;

¹H NMR (400 MHz, DMSO-d₆): δ=7.69 (d, 2H), 7.52 (d, 2H), 7.15 (t, 1H), 3.89-3.83 (m, 2H), 3.72 (d, 1H), 3.64 (d, 1H), 3.60-3.52 (m, 4H), 3.18-3.15 (m, 4H), 2.96-2.85 (m, 3H), 2.74 (t, 1H), 2.01-1.85 (m, 2H), 1.47-1.36 (m, 3H), 0.83 (t, 3H).

### Example 22

### {3-(Methoxymethyl)-5-[4-(trifluoromethyl)phenyl]piperidin-1-yl}(morpholin-4-yl)methanone [racemic cis isomer]

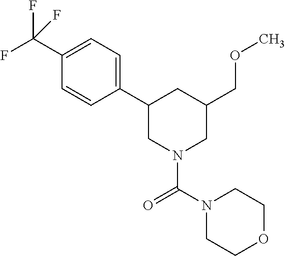

Under argon, 7 mg (2.22 mmol) of methanol were initially charged in 5.0 ml of N,N′-dimethylformamide, 3 Å molecular sieve and 27 mg (0.666 mmol, 60% in paraffin oil) of sodium hydride were added, and the mixture was stirred at RT for 30 min. Subsequently, 100 mg (0.222 mmol) of the mesylate from Example 7A in 1.0 ml of N,N′-dimethylformamide were added and the mixture was stirred at RT for 1 h. The reaction was ended by adding water, the molecular sieve was filtered off and the filtrate was purified by means of preparative HPLC. Yield: 59 mg (68% of theory)

LC-MS (Method 8B): Rₜ=1.08 min; MS (ESIpos): m/z=387 [M+H]⁺;

¹H NMR (400 MHz, DMSO-d₆): δ=7.68 (d, 2H), 7.52 (d, 2H), 3.73 (d, 1H), 3.62 (br s, 1H), 3.55 (t, 4H), 3.31-3.19 (m, 7H), 3.14 (d, 4H), 2.94-2.83 (m, 1H), 2.83-2.74 (m, 1H), 1.88 (d, 2H), 1.41 (q, 1H).

### Example 23

### {3-(Ethoxymethyl)-5-[4-(trifluoromethyl)phenyl]piperidin-1-yl}(morpholin-4-yl)methanone [racemic cis isomer]

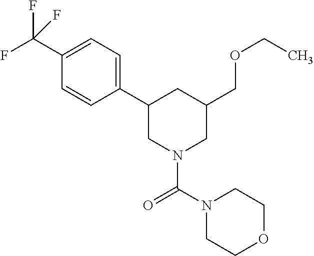

Under argon, 51 mg (1.11 mmol) of ethanol were initially charged in 2.0 ml of N,N′-dimethylformamide, 3 Å molecular sieve and 13 mg (0.333 mmol, 60% in paraffin oil) of sodium hydride were added, and the mixture was stirred at RT for 30 min. Subsequently, 50 mg (0.111 mmol) of the mesylate from Example 7A in 1.0 ml of N,N′-dimethylformamide were added and the mixture was stirred at RT for 2 h. The reaction was ended by adding water, the molecular sieve was filtered off and the filtrate was purified by means of preparative HPLC. Yield: 40.0 mg (87% of theory)

LC-MS (Method 8B): Rₜ=1.15 min; MS (ESIpos): m/z=401 [M+H]⁺;

¹H NMR (400 MHz, DMSO-d₆): δ=7.68 (d, 2H), 7.52 (d, 2H), 3.75 (d, 1H), 3.64 (d, 1H), 3.56 (br s, 4H), 3.41 (quin, 2H), 3.21-3.32 (m, 3H), 3.14 (br s, 4H), 2.94-2.83 (m, 1H), 2.84-2.73 (m, 1H), 1.89 (d, 2H), 1.40 (q, 1H), 1.11 (t, 3H).

### Example 24

### {3-(Isopropoxymethyl)-5-[4-(trifluoromethyl)phenyl]piperidin-1-yl}(morpholin-4-yl)methanone [racemic cis isomer]

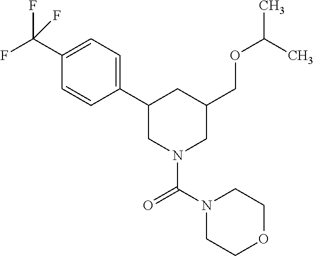

Under argon, 133 mg (2.22 mmol) of isopropanol were initially charged in 5.0 ml of N,N′-dimethylformamide, 3 Å molecular sieve and 27 mg (0.67 mmol, 60% in paraffin oil) of sodium hydride were added, and the mixture was stirred at RT for 30 min. Subsequently, 100 mg (0.22 mmol) of the mesylate from Example 7A in 1.0 ml of N,N′-dimethylformamide were added and the mixture was stirred at RT for 1 h. The reaction was ended by adding water, the molecular sieve was filtered off and the filtrate was purified by means of preparative HPLC. Yield: 32.8 mg (36% of theory)

LC-MS (Method 8B): Rₜ=1.21 min; MS (ESIpos): m/z=415 [M+H]⁺;

¹H NMR (400 MHz, DMSO-d₆): δ=7.68 (d, 2H), 7.51 (d, 2H), 3.75 (br s, 1H), 3.69-3.61 (m, 1H), 3.60-3.46 (m, 5H), 3.31-3.26 (m, 2H), 3.26-3.19 (m, 1H), 3.13 (d, 4H), 2.88 (br s, 1H), 2.83-2.73 (m, 1H), 1.87 (br s, 2H), 1.45-1.32 (m, 1H), 1.08 (d, 6H).

### Example 25

### {3-[(Cyclobutyloxy)methyl]-5-[4-(trifluoromethyl)phenyl]piperidin-1-yl}(morpholin-4-yl)methanone [racemic cis isomer]

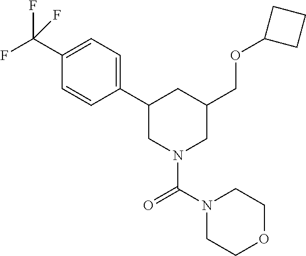

Under argon, 160 mg (2.22 mmol) of cyclobutanol were initially charged in 5.0 ml of N,N′-dimethylformamide, 3 Å molecular sieve and 27 mg (0.666 mmol, 60% in paraffin oil) of sodium hydride were added, and the mixture was stirred at RT for 30 min. Subsequently, 100 mg (0.222 mmol) of the mesylate from Example 7A in 1.0 ml of N,N′-dimethylformamide were added and the mixture was stirred at RT for 1 h. The reaction was ended by adding water, the molecular sieve was filtered off and the filtrate was purified by means of preparative HPLC. Yield: 63.8 mg (67% of theory)

LC-MS (Method 8B): Rₜ=1.25 min; MS (ESIpos): m/z=427 [M+H]⁺;

¹H NMR (400 MHz, DMSO-d₆): δ=7.68 (d, 2H), 7.51 (d, 2H), 3.87 (quin, 1H), 3.75 (d, 1H), 3.64 (d, 1H), 3.55 (d, 4H), 3.28 (d, 1H), 3.24-3.05 (m, 6H), 2.94-2.74 (m, 2H), 2.20-2.08 (m, 2H), 1.96-1.71 (m, 4H), 1.69-1.55 (m, 1H), 1.53-1.29 (m, 2H).

### Example 26

### {3-[(Cyclopentyloxy)methyl]-5-[4-(trifluoromethyl)phenyl]piperidin-1-yl}(morpholin-4-yl)methanone [racemic cis isomer]

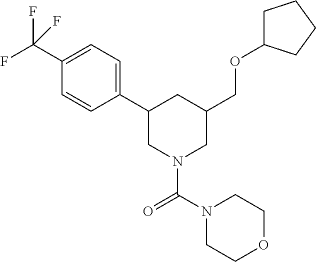

Under argon, 191 mg (2.22 mmol) of cyclopentanol were initially charged in 5.0 ml of N,N′-dimethylformamide, 3 Å molecular sieve and 27 mg (0.666 mmol, 60% in paraffin oil) of sodium hydride were added, and the mixture was stirred at RT for 30 min. Subsequently, 100 mg (0.222 mmol) of the mesylate from Example 7A in 1.0 ml of N,N′-dimethylformamide were added and the mixture was stirred at RT for 1 h. The reaction was ended by adding water, the molecular sieve was filtered off and the filtrate was purified by means of preparative HPLC. Yield: 33 mg (32% of theory)

LC-MS (Method 8B): Rₜ=1.13 min; MS (ESIpos): m/z=441 [M+H]⁺;

¹H NMR (400 MHz, DMSO-d₆): δ=7.68 (d, 2H), 7.51 (d, 2H), 3.84 (tt, 1H), 3.79-3.70 (m, 1H), 3.69-3.61 (m, 1H), 3.56 (t, 4H), 3.27 (dd, 2H), 3.22-3.09 (m, 5H), 2.86 (d, 1H), 2.82-2.70 (m, 1H), 1.87 (br s, 2H), 1.71-1.44 (m, 8H), 1.44-1.31 (m, 1H).

### Example 27

### {3-[(2-Methoxyethoxy)methyl]-5-[4-(trifluoromethyl)phenyl]piperidin-1-yl}(morpholin-4-yl)methanone [racemic cis isomer]

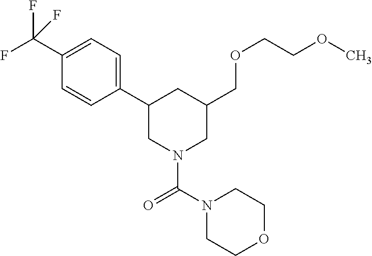

Under argon, 169 mg (2.22 mmol) of isopropanol were initially charged in 5.0 ml of N,N′-dimethylformamide, 3 Å molecular sieve and 27 mg (0.67 mmol, 60% in paraffin oil) of sodium hydride were added, and the mixture was stirred at RT for 30 min. Subsequently, 100 mg (0.22 mmol) of the mesylate from Example 7A in 1.0 ml of N,N′-dimethylformamide were added and the mixture was stirred at RT for 1 h. The reaction was ended by adding water, the molecular sieve was filtered off and the filtrate was purified by means of preparative HPLC. Yield: 59 mg (61% of theory).

LC-MS (Method 1B): Rₜ=1.22 min; MS (ESIpos): m/z=431 [M+H]⁺;

¹H NMR (400 MHz, DMSO-d₆): δ=7.68 (d, 2H), 7.52 (d, 2H), 3.76 (d, 1H), 3.64 (d, 1H), 3.60-3.40 (m, 8H), 3.25 (s, 3H), 3.14 (d, 4H), 2.95-2.83 (m, 1H), 2.83-2.73 (m, 1H), 1.88 (d, 2H), 1.39 (q, 1H); three protons hidden.

### Example 28

### Morpholin-4-yl{3-[(tetrahydro-2H-pyran-4-yloxy)methyl]-5-[4-(trifluoromethyl)phenyl]piperidin-1-yl}methanone [racemic cis isomer]

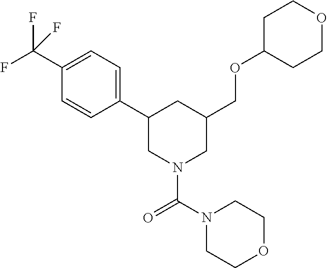

Under argon, 227 mg (2.22 mmol) of tetrahydro-2H-pyran-4-ol were initially charged in 5.0 ml of N,N′-dimethylformamide, 3 Å molecular sieve and 27 mg (0.666 mmol, 60% in paraffin oil) of sodium hydride were added, and the mixture was stirred at RT for 30 min. Subsequently, 100 mg (0.222 mmol) of the mesylate from Example 7A in 1.0 ml of N,N′-dimethylformamide were added and the mixture was stirred at RT for 1 h. The reaction was ended by adding water, the molecular sieve was filtered off and the filtrate was purified by means of preparative HPLC. Yield: 42 mg (42% of theory)

LC-MS (Method 7B): Rₜ=2.28 min; MS (ESIpos): m/z=457 [M+H]⁺;

¹H NMR (400 MHz, DMSO-d₆): δ=7.68 (d, 2H), 7.52 (d, 2H), 3.84-3.73 (m, 3H), 3.65 (d, 1H), 3.56 (t, 4H), 3.45 (tt, 1H), 3.40-3.23 (m, 5H), 3.14 (d, 4H), 2.95-2.84 (m, 1H), 2.83-2.74 (m, 1H), 1.96-1.77 (m, 4H), 1.48-1.31 (m, 7H).

### Example 29

### {3-[(Benzyloxy)methyl]-5-[4-(trifluoromethyl)phenyl]piperidin-1-yl}(morpholin-4-yl)methanone [racemic cis isomer]

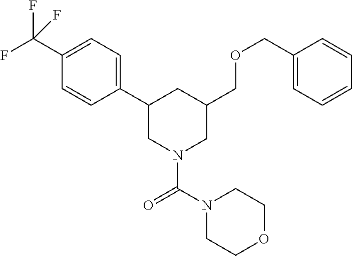

Under argon, 240 mg (2.22 mmol) of benzyl alcohol were initially charged in 5.0 ml of N,N′-dimethylformamide, 3 Å molecular sieve and 27 mg (0.666 mmol, 60% in paraffin oil) of sodium hydride were added, and the mixture was stirred at RT for 30 min. Subsequently, 100 mg (0.222 mmol) of the mesylate from Example 7A in 1.0 ml of N,N′-dimethylformamide were added and the mixture was stirred at RT for 1 h. The reaction was ended by adding water, the molecular sieve was filtered off and the filtrate was purified by means of preparative HPLC. Yield: 71.4 mg (70% of theory)

LC-MS (Method 7B): Rₜ=2.65 min; MS (ESIpos): m/z=463 [M+H]⁺;

¹H NMR (400 MHz, DMSO-d₆): δ=7.68 (d, 2H), 7.52 (d, 2H), 7.41-7.24 (m, 5H), 4.56-4.41 (m, 2H), 3.79 (d, 1H), 3.65 (d, 1H), 3.55 (t, 4H), 3.43-3.34 (m, 2H), 3.30 (s, 1H), 3.14 (d, 4H), 2.95-2.84 (m, 1H), 2.83-2.74 (m, 1H), 2.63-2.55 (m, 1H), 2.04-1.84 (m, 2H), 1.43 (q, 3H).

### Example 30

### {3-[(3-Fluorophenoxy)methyl]-5-[4-(trifluoromethyl)phenyl]piperidin-1-yl}(morpholin-4-yl)methanone [racemic cis isomer]

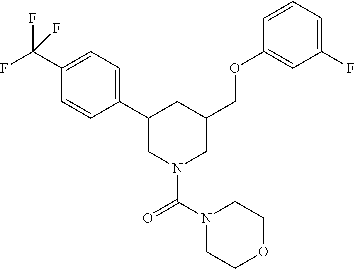

Under argon, 249 mg (2.22 mmol) of 3-fluorophenol were initially charged in 5.0 ml of N,N′-dimethylformamide, 3 Å molecular sieve and 27 mg (0.67 mmol, 60% in paraffin oil) of sodium hydride were added, and the mixture was stirred at RT for 30 min. Subsequently, 100 mg (0.22 mmol) of the mesylate from Example 7A in 1.0 ml of N,N′-dimethylformamide were added and the mixture was stirred at RT for 1 h. The reaction was ended by adding water, the molecular sieve was filtered off and the filtrate was purified by means of preparative HPLC. Yield: 87.1 mg (84% of theory)

LC-MS (Method 7B): Rₜ=2.69 min; MS (ESIpos): m/z=467 [M+H]⁺;

¹H NMR (400 MHz, DMSO-d₆): δ=7.70 (d, 2H), 7.54 (d, 2H), 7.32 (q, 1H), 6.89-6.71 (m, 3H), 4.02-3.94 (m, 1H), 3.94-3.80 (m, 2H), 3.67 (d, 1H), 3.56 (t, 4H), 3.21-3.08 (m, 4H), 3.01-2.90 (m, 1H), 2.88-2.78 (m, 1H), 2.73-2.62 (m, 1H), 2.13 (br s, 1H), 2.03 (d, 1H), 1.61-1.47 (m, 3H).

### Example 31

### {3-[(Isopropylsulphanyl)methyl]-5-[4-(trifluoromethyl)phenyl]piperidin-1-yl}(morpholin-4-yl)methanone [racemic cis isomer]

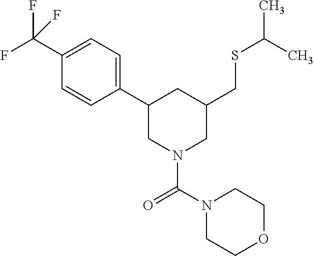

Under argon, 423 mg (5.55 mmol) of propane-2-thiol were initially charged in 10.0 ml of N,N′-dimethylformamide, 3 Å molecular sieve and 67 mg (1.67 mmol, 60% in paraffin oil) of sodium hydride were added, and the mixture was stirred at RT for 30 min. Subsequently, 250 mg (0.555 mmol) of the mesylate from Example 7A in 5.0 ml of N,N′-dimethylformamide were added and the mixture was stirred at RT for 3 h. The reaction was ended by adding water, the molecular sieve was filtered off and the filtrate was purified by means of preparative HPLC. Yield: 198 mg (80% of theory)

LC-MS (Method 8B): Rₜ=1.28 min; MS (ESIpos): m/z=431 [M+H]⁺;

¹H NMR (400 MHz, DMSO-d₆): δ=7.68 (d, 2H), 7.51 (d, 2H), 3.85 (d, 1H), 3.64 (d, 1H), 3.57 (t, 4H), 3.15 (d, 4H), 2.97-2.84 (m, 2H), 2.83-2.73 (m, 1H), 2.48-2.35 (m, 2H), 2.03 (d, 1H), 1.77 (br s, 1H), 1.41 (q, 1H), 1.21 (d, 6H); one proton hidden.

### Example 32

### {3-[(Ethylsulphanyl)methyl]-5-[4-(trifluoromethyl)phenyl]piperidin-1-yl}(morpholin-4-yl)methanone [racemic cis isomer]

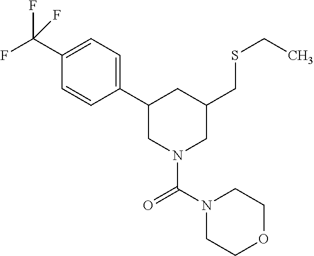

Under argon, 690 mg (11.1 mmol) of ethanethiol were initially charged in 25.0 ml of N,N′-dimethylformamide, 3 Å molecular sieve and 133 mg (3.33 mmol, 60% in paraffin oil) of sodium hydride were added, and the mixture was stirred at RT for 30 min. Subsequently, 500 mg (1.11 mmol) of the mesylate from Example 7A in 5.0 ml of N,N′-dimethylformamide were added and the mixture was stirred at RT for 3 h. The reaction was ended by adding water, the molecular sieve was filtered off and the filtrate was purified by means of preparative HPLC. Yield: 223 mg (48% of theory)

LC-MS (Method 1B): Rₜ=1.38 min; MS (ESIpos): m/z=417 [M+H]⁺;

¹H NMR (400 MHz, DMSO-d₆): δ=7.69 (d, 2H), 7.51 (d, 2H), 3.85 (d, 1H), 3.64 (d, 1H), 3.57 (t, 4H), 3.15 (d, 4H), 2.95-2.84 (m, 1H), 2.83-2.73 (m, 1H), 2.49-2.39 (m, 3H), 2.02 (d, 1H), 1.79 (br s, 1H), 1.41 (q, 1H), 1.18 (t, 3H); two protons hidden.

### Example 33

### {3-[(Isopropylsulphinyl)methyl]-5-[4-(trifluoromethyl)phenyl]piperidin-1-yl}(morpholin-4-yl)methanone [racemic cis diastereomer mixture]

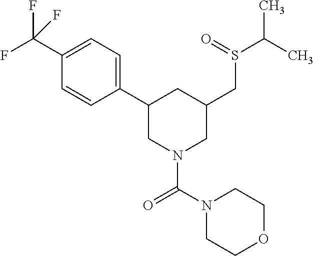

140 mg (0.325 mmol) of the thioether from Example 31 in 14.0 ml of dichloromethane were admixed with 168 mg (0.488 mmol, 50%) of meta-chloroperoxybenzoic acid, and the mixture was stirred at RT for 1 h. The reaction solution was concentrated under reduced pressure and the residue was purified by means of preparative HPLC. Yield: 53 mg (36% of theory)

LC-MS (Method 8B): Rₜ=0.97 min; MS (ESIpos): m/z=447 [M+H]⁺;

¹H NMR (400 MHz, DMSO-d₆): δ=7.70 (d, 2H), 7.53 (d, 2H), 3.92-3.75 (m, 1H), 3.65 (d, 1H), 3.57 (t, 4H), 3.21-3.11 (m, 4H), 3.03-2.89 (m, 1H), 2.88-2.77 (m, 2H), 2.70-2.59 (m, 2H), 2.57 (d, 2H), 2.57-2.55 (m, 1H), 2.19-2.00 (m, 2H), 1.56 (quin, 1H), 1.23-1.12 (m, 6H).

### Example 34

### {3-[(Isopropylsulphonyl)methyl]-5-[4-(trifluoromethyl)phenyl]piperidin-1-yl}(morpholin-4-yl)methanone [racemic cis isomer]

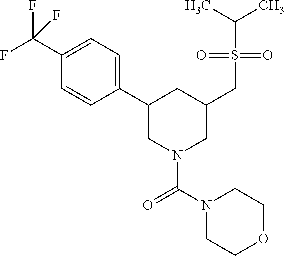

140 mg (0.325 mmol) of the thioether from Example 31 in 14.0 ml of dichloromethane were admixed with 168 mg (0.488 mmol, 50%) of meta-chloroperoxybenzoic acid, and the mixture was stirred at RT for 1 h. The reaction solution was concentrated under reduced pressure and the residue was purified by means of preparative HPLC. Yield: 79 mg (52% of theory)

LC-MS (Method 8B): Rₜ=1.03 min; MS (ESIpos): m/z=463 [M+H]⁺;

¹H NMR (400 MHz, DMSO-d₆): δ=7.70 (d, 2H), 7.52 (d, 2H), 3.96 (d, 1H), 3.65 (d, 1H), 3.56 (d, 4H), 3.30-3.21 (m, 2H), 3.19-3.05 (m, 6H), 3.02-2.90 (m, 1H), 2.85-2.73 (m, 1H), 2.65 (t, 1H), 2.33-2.21 (m, 1H), 2.10 (d, 1H), 1.56 (q, 1H), 1.25 (d, 6H); one proton hidden.

### Example 35

### {3-[(Ethylsulphinyl)methyl]-5-[4-(trifluoromethyl)phenyl]piperidin-1-yl}(morpholin-4-yl)methanone [racemic cis diastereomer mixture]

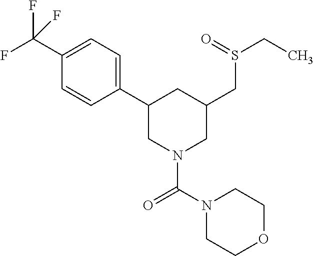

160 mg (0.384 mmol) of the thioether from Example 32 in 16.0 ml of dichloromethane were admixed with 199 mg (0.576 mmol, 50%) of meta-chloroperoxybenzoic acid, and the mixture was stirred at RT for 45 min. The reaction solution was concentrated under reduced pressure and the residue was purified by means of preparative HPLC. Yield: 60 mg (34% of theory)

LC-MS (Method 1B): Rₜ=1.04 min; MS (ESIpos): m/z=433 [M+H]⁺;

¹H NMR (400 MHz, DMSO-d₆): δ=7.69 (d, 2H), 7.53 (d, 2H), 3.91-3.76 (m, 1H), 3.64 (d, 1H), 3.57 (t, 4H), 3.16 (d, 4H), 3.03-2.88 (m, 1H), 2.88-2.74 (m, 2H), 2.75-2.56 (m, 4H), 2.21-1.98 (m, 2H), 1.55 (quin, 1H), 1.19 (dt, 3H).

### Example 36

### {3-[(Ethylsulphonyl)methyl]-5-[4-(trifluoromethyl)phenyl]piperidin-1-yl}(morpholin-4-yl)methanone [racemic cis isomer]

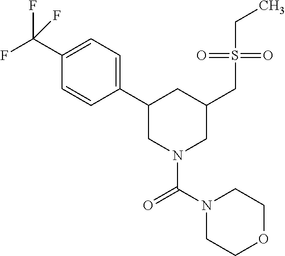

160 mg (0.384 mmol) of the thioether from Example 32 in 16.0 ml of dichloromethane were admixed with 199 mg (0.576 mmol, 50%) of meta-chloroperoxybenzoic acid, and the mixture was stirred at RT for 45 min. The reaction solution was concentrated under reduced pressure and the residue was purified by means of preparative HPLC. Yield: 83 mg (48% of theory)

LC-MS (Method 1B): Rₜ=1.11 min; MS (ESIpos): m/z=449 [M+H]⁺;

¹H NMR (400 MHz, DMSO-d₆): δ=7.70 (d, 2H), 7.52 (d, 2H), 3.95 (d, 1H), 3.64 (d, 1H), 3.56 (d, 4H), 3.21-3.06 (m, 8H), 3.02-2.88 (m, 1H), 2.86-2.75 (m, 1H), 2.69-2.58 (m, 1H), 2.26 (br s, 1H), 2.13-2.03 (m, 1H), 1.55 (q, 1H), 1.23 (t, 6H).

### Example 37

### Morpholin-4-yl{3-[(phenylsulphanyl)methyl]-5-[4-(trifluoromethyl)phenyl]piperidin-1-yl}methanone [racemic cis isomer]

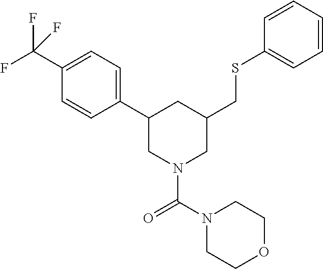

Under argon, 618 mg (5.55 mmol) of thiophenol were initially charged in 10.0 ml N,N′-dimethylformamide, 3 Å molecular sieve and 67 mg (1.67 mmol, 60% in paraffin oil) of sodium hydride were added, and the mixture was stirred at RT for 30 min. Subsequently, 250 mg (0.555 mmol) of the mesylate from Example 7A in 5.0 ml of N,N′-dimethylformamide were added and the mixture was stirred at RT for 3 h. The reaction was ended by adding water, the molecular sieve was filtered off and the filtrate was purified by means of preparative HPLC. Yield: 211 mg (82% of theory)

LC-MS (Method 1B): Rₜ=1.46 min; MS (ESIpos): m/z=465 [M+H]⁺;

¹H NMR (400 MHz, DMSO-d₆): δ=7.68 (d, 2H), 7.51 (d, 2H), 7.41-7.27 (m, 4H), 7.25-7.15 (m, 1H), 3.88 (d, 1H), 3.63 (d, 1H), 3.53 (t, 4H), 3.16-2.97 (m, 5H), 2.95-2.72 (m, 3H), 2.57 (s, 1H), 2.15-1.99 (m, 1H), 1.82 (br s, 1H), 1.49 (q, 1H).

### Example 38

### {3-[(Benzylsulphanyl)methyl]-5-[4-(trifluoromethyl)phenyl]piperidin-1-yl}(morpholin-4-yl)methanone [racemic cis isomer]

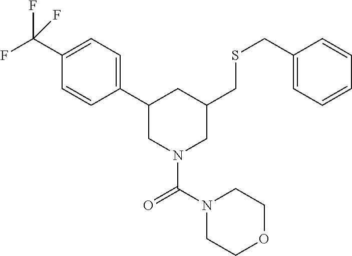

Under argon, 689 mg (5.55 mmol) of benzylthiol were initially charged in 10.0 ml N,N′-dimethylformamide, 3 Å molecular sieve and 67 mg (1.67 mmol, 60% in paraffin oil) of sodium hydride were added, and the mixture was stirred at RT for 30 min. Subsequently, 250 mg (0.555 mmol) of the mesylate from Example 7A in 5.0 ml of N,N′-dimethylformamide were added and the mixture was stirred at RT for 3 h. The reaction was ended by adding water, the molecular sieve was filtered off and the filtrate was purified by means of preparative HPLC. Yield: 137 mg (52% of theory)

LC-MS (Method 8B): Rₜ=1.33 min; MS (ESIpos): m/z=479 [M+H]⁺;

¹H NMR (400 MHz, DMSO-d₆): δ=7.68 (d, 2H), 7.50 (d, 2H), 7.39-7.17 (m, 5H), 3.83-3.71 (m, 3H), 3.68-3.50 (m, 5H), 3.14 (d, 4H), 2.83 (d, 1H), 2.78-2.69 (m, 1H), 2.48-2.26 (m, 3H), 1.96 (d, 1H), 1.76 (br s, 1H), 1.36 (q, 1H).

### Example 39

### {3-[(Cyclohexylsulphanyl)methyl]-5-[4-(trifluoromethyl)phenyl]piperidin-1-yl}(morpholin-4-yl)methanone [racemic cis isomer]

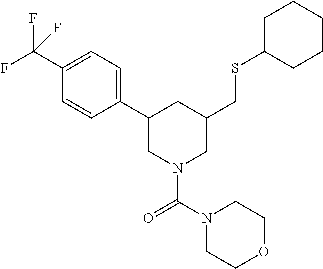

Under argon, 645 mg (5.55 mmol) of cyclohexyl thiol were initially charged in 8.0 ml N,N′-dimethylformamide, 3 Å molecular sieve and 67 mg (1.67 mmol, 60% in paraffin oil) of sodium hydride were added, and the mixture was stirred at RT for 30 min. Subsequently, 250 mg (0.555 mmol) of the mesylate from Example 7A in 2.0 ml of N,N′-dimethylformamide were added and the mixture was stirred at RT for 3 h. The reaction was ended by adding water, the molecular sieve was filtered off and the filtrate was purified by means of preparative HPLC. Yield: 166 mg (64% of theory)

LC-MS (Method 7B): Rₜ=2.92 min; MS (ESIpos): m/z=471 [M+H]⁺;

¹H NMR (400 MHz, DMSO-d₆): δ=7.68 (d, 2H), 7.51 (d, 2H), 3.86 (d, 1H), 3.64 (d, 1H), 3.57 (t, 4H), 3.14 (br s, 4H), 2.94-2.83 (m, 1H), 2.83-2.73 (m, 1H), 2.67 (d, 1H), 2.48-2.31 (m, 2H), 2.02 (d, 1H), 1.91 (br s, 2H), 1.81-1.62 (m, 3H), 1.55 (d, 1H), 1.40 (q, 1H), 1.30-1.17 (m, 4H).

### Example 40

### (3-{[(3-Fluorophenyl)sulphanyl]methyl}-5-[4-(trifluoromethyl)phenyl]piperidin-1-yl)(morpholin-4-yl)methanone [racemic cis isomer]

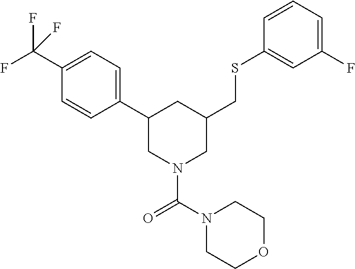

Under argon, 748 mg (5.55 mmol) of 3-fluorothiophenol were initially charged in 8.0 ml N,N′-dimethylformamide, 3 Å molecular sieve and 67 mg (1.67 mmol, 60% in paraffin oil) of sodium hydride were added, and the mixture was stirred at RT for 30 min. Subsequently, 250 mg (0.555 mmol) of the mesylate from Example 7A in 2.0 ml of N,N′-dimethylformamide were added and the mixture was stirred at RT for 3 h. The reaction was ended by adding water, the molecular sieve was filtered off and the filtrate was purified by means of preparative HPLC. Yield: 220 mg (82% of theory)

LC-MS (Method 7B): Rₜ=2.73 min; MS (ESIpos): m/z=483 [M+H]⁺;

¹H NMR (400 MHz, DMSO-d₆): δ=7.69 (d, 2H), 7.51 (d, 2H), 7.42-7.30 (m, 1H), 7.23 (d, 1H), 7.18 (d, 1H), 7.01 (dt, 1H), 3.86 (d, 1H), 3.62 (d, 1H), 3.53 (t, 4H), 3.14-3.03 (m, 5H), 3.01-2.93 (m, 1H), 2.92-2.83 (m, 1H), 2.83-2.74 (m, 1H), 2.62-2.55 (m, 1H), 2.14-2.03 (m, 1H), 1.83 (br s, 1H), 1.49 (q, 1H).

### Example 41

### Morpholin-4-yl{3-[(phenylsulphinyl)methyl]-5-[4-(trifluoromethyl)phenyl]piperidin-1-yl}methanone [racemic cis diastereomer mixture]

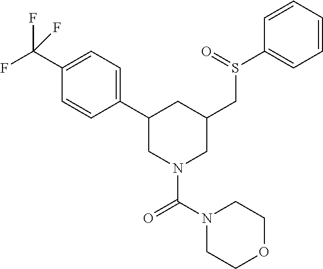

160 mg (0.344 mmol) of the thioether from Example 37 in 15.0 ml of dichloromethane were admixed with 178 mg (0.576 mmol, 50%) of meta-chloroperoxybenzoic acid, and the mixture was stirred at RT for 45 min. The reaction solution was concentrated under reduced pressure and the residue was purified by means of preparative HPLC. Yield: 60 mg (36% of theory)

LC-MS (Method 8B): Rₜ=1.05 min; MS (ESIpos): m/z=481 [M+H]⁺.

### Example 42

### Morpholin-4-yl{3-[(phenylsulphonyl)methyl]-5-[4-(trifluoromethyl)phenyl]piperidin-1-yl}methanone [racemic cis isomer]

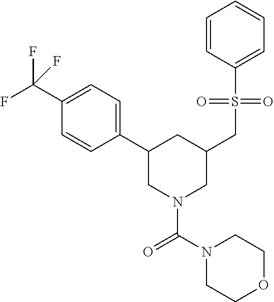

160 mg (0.344 mmol) of the thioether from Example 37 in 15.0 ml of dichloromethane were admixed with 178 mg (0.576 mmol, 50%) of meta-chloroperoxybenzoic acid, and the mixture was stirred at RT for 45 min. The reaction solution was concentrated under reduced pressure and the residue was purified by means of preparative HPLC. Yield: 83 mg (48% of theory)

LC-MS (Method 8B): Rₜ=1.09 min; MS (ESIpos): m/z=497 [M+H]⁺;

¹H NMR (500 MHz, DMSO-d₆): δ=7.94 (d, 2H), 7.76 (d, 1H), 7.72-7.65 (m, 4H), 7.47 (d, 2H), 3.93 (d, 1H), 3.66-3.52 (m, 5H), 3.47-3.28 (m, 2H), 3.13 (d, 4H), 2.94-2.82 (m, 1H), 2.81-2.72 (m, 1H), 2.64 (t, 1H), 2.00 (d, 2H), 1.52 (q, 1H).

### Example 43

### {3-[(Cyclohexylsulphinyl)methyl]-5-[4-(trifluoromethyl)phenyl]piperidin-1-yl}(morpholin-4-yl)methanone [racemic cis diastereomer mixture]

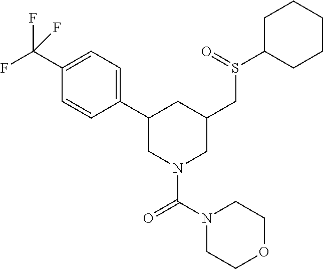

120 mg (0.255 mmol) of the thioether from Example 39 in 11.0 ml of dichloromethane were admixed with 132 mg (0.382 mmol, 50%) of meta-chloroperoxybenzoic acid, and the mixture was stirred at RT for 45 min. The reaction solution was concentrated under reduced pressure and the residue was purified by means of preparative HPLC. Yield: 32 mg (26% of theory)

LC-MS (Method 8B): Rₜ=1.09 and 1.10 min; MS (ESIpos): m/z=487 [M+H]⁺.

### Example 44

### {3-[(Cyclohexylsulphonyl)methyl]-5-[4-(trifluoromethyl)phenyl]piperidin-1-yl}(morpholin-4-yl)methanone [racemic cis isomer]

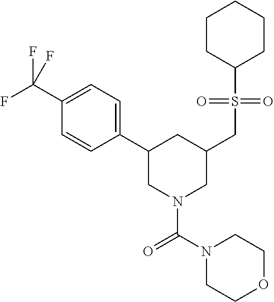

120 mg (0.255 mmol) of the thioether from Example 39 in 11.0 ml of dichloromethane were admixed with 132 mg (0.382 mmol, 50%) of meta-chloroperoxybenzoic acid, and the mixture was stirred at RT for 45 min. The reaction solution was concentrated under reduced pressure and the residue was purified by means of preparative HPLC. Yield: 59 mg (46% of theory)

LC-MS (Method 8B): Rₜ=1.16 min; MS (ESIpos): m/z=503 [M+H]⁺;

¹H NMR (500 MHz, DMSO-d₆): δ=7.69 (d, 2H), 7.51 (d, 2H), 3.96 (d, 1H), 3.65 (d, 1H), 3.57 (t, 4H), 3.15 (d, 4H), 3.10-3.01 (m, 3H), 3.00-2.91 (m, 1H), 2.83-2.73 (m, 1H), 2.64 (t, 1H), 2.27 (br s, 1H), 2.16-2.01 (m, 3H), 1.82 (d, 2H), 1.64 (d, 1H), 1.55 (q, 1H), 1.44-1.22 (m, 4H), 1.21-1.09 (m, 1H).

### Example 45

### (3-{[(3-Fluorophenyl)sulphinyl]methyl}-5-[4-(trifluoromethyl)phenyl]piperidin-1-yl)(morpholin-4-yl)methanone [racemic cis diastereomer mixture]

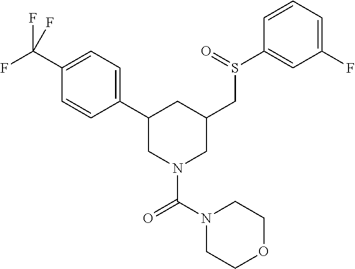

160 mg (0.332 mmol) of the thioether from Example 40 in 14.0 ml of dichloromethane were admixed with 172 mg (0.576 mmol, 50%) of meta-chloroperoxybenzoic acid, and the mixture was stirred at RT for 45 min. The reaction solution was concentrated under reduced pressure and the residue was purified by means of preparative HPLC. Yield: 65 mg (40% of theory)

LC-MS (Method 1B): Rₜ=1.22 min; MS (ESIpos): m/z=499 [M+H]⁺.

### Example 46

### (3-{[(3-Fluorophenyl)sulphonyl]methyl}-5-[4-(trifluoromethyl)phenyl]piperidin-1-yl)(morpholin-4-yl)methanone [racemic cis isomer]

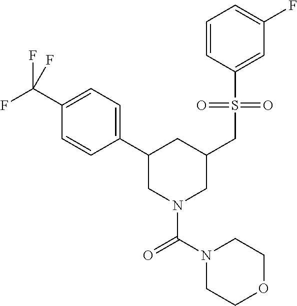

160 mg (0.332 mmol) of the thioether from Example 40 in 14.0 ml of dichloromethane were admixed with 172 mg (0.576 mmol, 50%) of meta-chloroperoxybenzoic acid, and the mixture was stirred at RT for 45 min. The reaction solution was concentrated under reduced pressure and the residue was purified by means of preparative HPLC. Yield: 55 mg (32% of theory)

LC-MS (Method 1B): Rₜ=1.27 min; MS (ESIpos): m/z=515 [M+H]⁺;

¹H NMR (500 MHz, DMSO-d₆): δ=7.83-7.72 (m, 3H), 7.71-7.60 (m, 3H), 7.48 (d, 2H), 3.92 (d, 1H), 3.65-3.53 (m, 5H), 3.51-3.36 (m, 2H), 3.13 (d, 4H), 2.96-2.84 (m, 1H), 2.82-2.73 (m, 1H), 2.64 (t, 1H), 2.14-1.98 (m, 3H), 1.53 (q, 4H).

### Example 47

### {3-[(Cyclopentylsulphanyl)methyl]-5-[4-(trifluoromethyl)phenyl]piperidin-1-yl}(morpholin-4-yl)methanone [racemic cis isomer]

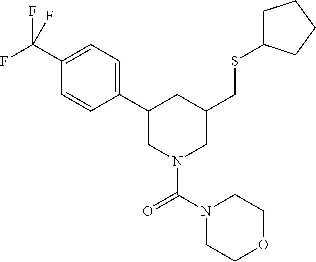

Under argon, 749 mg (7.33 mmol) of cyclopentylthiol were initially charged in 11.0 ml N,N′-dimethylformamide, 3 Å molecular sieve and 88 mg (2.20 mmol, 60% in paraffin oil) of sodium hydride were added, and the mixture was stirred at RT for 30 min. Subsequently, 330 mg (0.733 mmol) of the mesylate from Example 7A in 2.0 ml of N,N′-dimethylformamide were added and the mixture was stirred at RT for 3 h. The reaction was ended by adding water, the molecular sieve was filtered off and the filtrate was purified by means of preparative HPLC. Yield: 83 mg (25% of theory)

LC-MS (Method 7B): Rₜ=2.82 min; MS (ESIpos): m/z=457 [M+H]⁺;

¹H NMR (400 MHz, DMSO-d₆): δ=7.68 (d, 2H), 7.51 (d, 2H), 3.86 (d, 1H), 3.64 (d, 1H), 3.57 (t, 4H), 3.20-3.05 (m, 6H), 2.94-2.84 (m, 1H), 2.83-2.74 (m, 1H), 2.48-2.36 (m, 3H), 2.07-1.88 (m, 3H), 1.79 (br s, 1H), 1.72-1.61 (m, 2H), 1.60-1.48 (m, 2H), 1.47-1.34 (m, 3H).

### Example 48

### {3-[(Cyclopentylsulphonyl)methyl]-5-[4-(trifluoromethyl)phenyl]piperidin-1-yl}(morpholin-4-yl)methanone [racemic cis isomer]

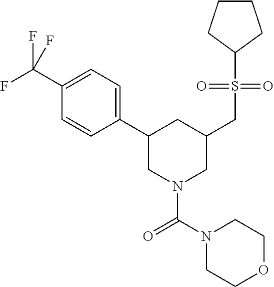

40 mg (0.088 mmol) of the thioether from Example 47 in 4.0 ml of dichloromethane were admixed with 61 mg (0.175 mmol, 50%) of meta-chloroperoxybenzoic acid, and the mixture was stirred at RT for 45 min The reaction solution was concentrated under reduced pressure and the residue was purified by means of preparative HPLC. Yield: 37 mg (87% of theory)

LC-MS (Method 8B): Rₜ=1.08 min; MS (ESIpos): m/z=489 [M+H]⁺;

¹H NMR (400 MHz, DMSO-d₆): δ=7.70 (d, 2H), 7.51 (d, 2H), 3.96 (d, 1H), 3.69-3.51 (m, 6H), 3.14 (br s, 4H), 3.11-3.04 (m, 1H), 2.83-2.74 (m, 1H), 2.63 (d, 1H), 2.07 (s, 6H), 1.90 (br s, 3H), 1.71-1.53 (m, 4H).

### Example 49

### {3-[(Benzylsulphinyl)methyl]-5-[4-(trifluoromethyl)phenyl]piperidin-1-yl}(morpholin-4-yl)methanone [racemic cis diastereomer mixture]

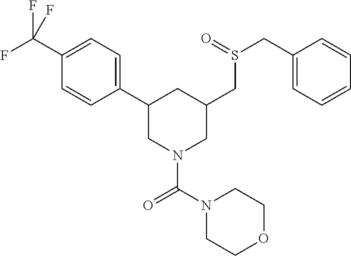

100 mg (0.209 mmol) of the thioether from Example 38 in 9.0 ml of dichloromethane were admixed with 108 mg (0.315 mmol, 50%) of meta-chloroperoxybenzoic acid, and the mixture was stirred at RT for 45 min. The reaction solution was concentrated under reduced pressure and the residue was purified by means of preparative HPLC. Yield: 41 mg (38% of theory)

LC-MS (Method 7B): Rₜ=2.23 min; MS (ESIpos): m/z=495 [M+H]⁺;

¹H NMR (400 MHz, DMSO-d₆): δ=7.68 (d, 2H), 7.52 (d, 2H), 7.43-7.28 (m, 5H), 4.17 (dd, 1H), 4.00 (d, 1H), 3.80 (t, 1H), 3.63 (d, 1H), 3.56 (br s, 4H), 3.21-3.08 (m, 4H), 3.01-2.76 (m, 2H), 2.75-2.57 (m, 3H), 2.23-1.87 (m, 2H), 1.64-1.44 (m, 1H).

### Example 50

### {3-[(Benzylsulphonyl)methyl]-5-[4-(trifluoromethyl)phenyl]piperidin-1-yl}(morpholin-4-yl)methanone [racemic cis isomer]

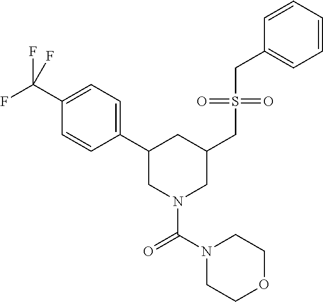

100 mg (0.209 mmol) of the thioether from Example 38 in 9.0 ml of dichloromethane were admixed with 108 mg (0.315 mmol, 50%) of meta-chloroperoxybenzoic acid, and the mixture was stirred at RT for 45 min. The reaction solution was concentrated under reduced pressure and the residue was purified by means of preparative HPLC. Yield: 37 mg (34% of theory)

LC-MS (Method 7B): Rₜ=2.36 min; MS (ESIpos): m/z=511 [M+H]⁺;

¹H NMR (400 MHz, DMSO-d₆): δ=7.69 (d, 2H), 7.50 (d, 2H), 7.44-7.35 (m, 5H), 4.58-4.46 (m, 2H), 3.90 (d, 1H), 3.62 (d, 1H), 3.56 (d, 4H), 3.13 (br. s., 4H), 3.07 (d, 2H), 2.96-2.85 (m, 1H), 2.80-2.71 (m, 1H), 2.66-2.56 (m, 1H), 2.22 (br s, 1H), 2.06-1.94 (m, 1H), 1.53 (q, 1H).

### Example 51

### (3-{[(2-Methoxyethyl)(methyl)amino]methyl}-5-[4-(trifluoromethyl)phenyl]piperidin-1-yl)(morpholin-4-yl)methanone [racemic cis isomer]

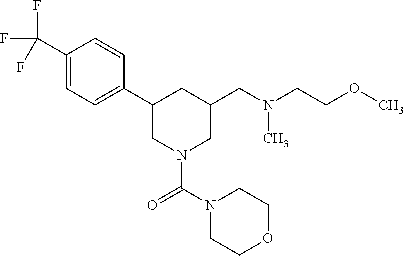

According to General Method 7, 300 mg (approx. 0.333 mmol) of the compound from Example 7A and 356 mg (3.996 mmol) of methoxyethanamine were reacted. Yield: 63 mg (40% of theory)

HPLC (Method 8B): Rₜ=0.80 min; MS (ESIpos): m/z=444 [M+H]⁺.

### Example 52

### (3-{[Cyclopropyl(methyl)amino]methyl}-5-[4-(trifluoromethyl)phenyl]piperidin-1-yl)(morpholin-4-yl)methanone [racemic cis isomer]

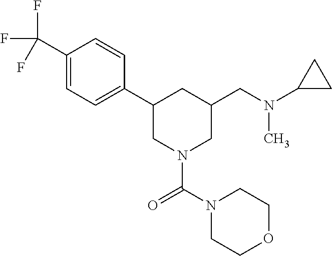

According to General Method 8, 300 mg (approx. 0.333 mmol) of the compound from Example 7A and 341 mg (4.794 mmol) of N-methylcyclopropanamine were reacted. Yield: 29 mg (17% of theory)

HPLC (Method 8B): Rₜ=0.80 min; MS (ESIpos): m/z=426 [M+H]⁺.

### Example 53

### {3-[(3-Hydroxypyrrolidin-1-yl)methyl]-5-[4-(trifluoromethyl)phenyl]piperidin-1-yl}(morpholin-4-yl)methanone [racemic cis isomer]

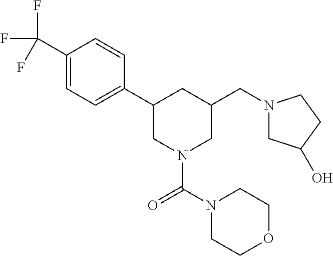

According to General Method 8, 300 mg (approx. 0.333 mmol) of the compound from Example 7A and 292 mg (4.794 mmol) of 3-pyrrolidinol were reacted. Yield: 79 mg (45% of theory)

HPLC (Method 8B): Rₜ=0.75 min; MS (ESIpos): m/z=442 [M+H]⁺.

### Example 54

### {1-[(1,1-Dioxidothiomorpholin-4-yl)carbonyl]-5-[4-(trifluoromethyl)phenyl]piperidin-3-yl}methyl propylcarbamate [racemic cis isomer]

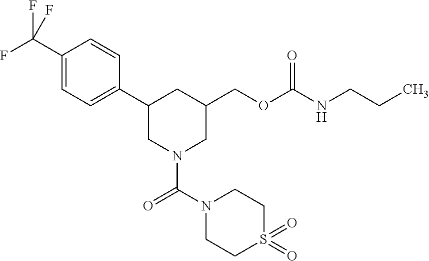

According to General Method 5, 115 mg (0.120 mmol) of the compound from Example 16A and 12 mg (0.144 mmol) of isobutyl isocyanate were reacted. Yield: 19 mg (29% of theory)

LC-MS (Method 8B): Rₜ=1.0 min; MS (ESIpos): m/z=506 [M+H]⁺;

¹H NMR (500 MHz, DMSO-d₆): δ=7.69 (d, 2H), 7.52 (d, 1H), 7.15-7.13 (m, 1H), 3.95-3.82 (m, 2H), 3.78-3.66 (m, 2H), 3.60-3.54 (m, 3H), 3.19-3.12 (m, 3H), 2.95-2.78 (m, 4H), 1.92 (m, 2H), 1.46-1.35 (m, 3H), 0.82 (t, 3H).

### Example 55

### N-({1-(Morpholin-4-ylcarbonyl)-5-[4-(trifluoromethyl)phenyl]piperidin-3-yl}methyl)cyclopropanecarboxamide [enantiomerically pure cis isomer]

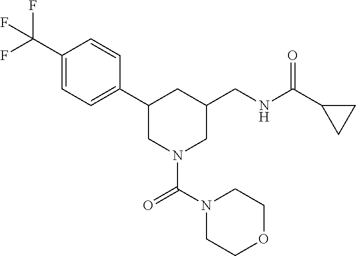

According to General Method 2, 129 mg (approx. 0.219 mmol) of {3-(aminomethyl)-5-[4-(trifluoromethyl)phenyl]piperidin-1-yl}(morpholin-4-yl)methanone were reacted. Enantiomer separation of the racemate by Method 5D gave 13 mg of the title compound from Example 55 and 13 mg of the title compound from Example 56.

LC-MS (Method 8B): Rₜ=0.97 min; MS (ESIpos): m/z=440 [M+H]⁺;

HPLC (Method 4E): Rₜ=6.67 min, >99.0% ee;

¹H NMR (400 MHz, DMSO-d₆): δ=8.16-8.13 (m, 1H), 7.68 (d, 2H), 7.52 (d, 2H), 3.68-3.62 (m, 2H), 3.60-3.55 (m, 4H), 3.17-3.07 (m, 4H), 3.04-2.99 (m, 2H), 2.89-2.71 (m, 2H); 1.89 (d, 1H), 1.80-1.65 (m, 1H), 1.57-1.50 (m, 1H), 1.37 (q, 1H), 0.70-0.60 (m, 4H).

### Example 56

### N-({1-(Morpholin-4-ylcarbonyl)-5-[4-(trifluoromethyl)phenyl]piperidin-3-yl}methyl)cyclopropanecarboxamide [enantiomerically pure cis isomer]

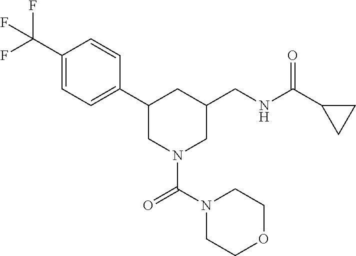

According to General Method 2, 129 mg (approx. 0.219 mmol) of {3-(aminomethyl)-5-[4-(trifluoromethyl)phenyl]piperidin-1-yl}(morpholin-4-yl)methanone were reacted. Enantiomer separation of the racemate by Method 5D gave 13 mg of the title compound from Example 55 and 13 mg of the title compound from Example 56.

LC-MS (Method 8B): Rₜ=0.97 min; MS (ESIpos): m/z=440 [M+H]⁺;

HPLC (Method 4E): Rₜ=7.14 min, >85.0% ee;

¹H NMR (400 MHz, DMSO-d₆): δ=8.16-8.13 (m, 1H), 7.68 (d, 2H), 7.52 (d, 2H), 3.68-3.62 (m, 2H), 3.60-3.55 (m, 4H), 3.17-3.07 (m, 4H), 3.04-2.99 (m, 2H), 2.89-2.71 (m, 2H); 1.89 (d, 1H), 1.80-1.65 (m, 1H), 1.57-1.50 (m, 1H), 1.37 (q, 1H), 0.70-0.60 (m, 4H).

### B) ASSESSMENT OF PHYSIOLOGICAL ACTIVITY

### Abbreviations

BSA bovine serum albumin
DMEM Dulbecco's Modified Eagle Medium
EGTA ethylene glycol-bis(2-aminoethyl)-N,N,N′,N′-tetraacetic acid
FCS fetal calf serum
HEPES 4-(2-hydroxyethyl)-1-piperazineethanesulphonic acid
[3H]haTRAP tritiated high affinity thrombin receptor activating peptide
PRP platelet-rich plasma

The suitability of the inventive compounds for treating thromboembolic disorders can be demonstrated in the following assay systems:

### 1.) In Vitro Assays

### 1.a) Cellular Functional In Vitro Test

A recombinant cell line is used to identify antagonists of the human protease activated receptor 1 (PAR-1) and to quantify the activity of the substances described herein. The cell is originally derived from a human embryonal kidney cell (HEK293; ATCC: American Type Culture Collection, Manassas, Va. 20108, USA). The test cell line constitutively expresses a modified form of the calcium-sensitive photoprotein aequorin which, after reconstitution with the cofactor coelenterazine, emits light when the free calcium concentration in the inner mitochondrial compartment is increased (Rizzuto R, Simpson A W, Brini M, Pozzan T.; Nature 1992, 358, 325-327). Additionally, the cell stably expresses the endogenous human PAR-1 receptor and the endogenous purinergic receptor P2Y2. The resulting PAR-1 test cell responds to stimulation of the endogenous PAR-1 or P2Y2 receptor with an intracellular release of calcium ions, which can be quantified through the resulting aequorin luminescence with a suitable luminometer (Milligan G, Marshall F, Rees S, Trends in Pharmacological Sciences 1996, 17, 235-237).

For the testing of the substance specificity, the effect thereof after activation of the endogenous PAR-1 receptor is compared with the effect after activation of the endogenous purinergic P2Y2 receptor which utilizes the same intracellular signal path.

Test procedure: The cells are plated out two days (48 h) before the test in culture medium (DMEM F12, supplemented with 10% FCS, 2 mM glutamine, 20 mM HEPES, 1.4 mM pyruvate, 0.1 mg/ml gentamycin, 0.15% Na bicarbonate; BioWhittaker Cat.# BE04-687Q; B-4800 Verviers, Belgium) in 384-well microtitre plates and kept in a cell incubator (96% atmospheric humidity, 5% v/v CO₂, 37° C.). On the day of the test, the culture medium is replaced by a tyrode solution (in mM: 140 sodium chloride, 5 potassium chloride, 1 magnesium chloride, 2 calcium chloride, 20 glucose, 20 HEPES), which additionally contains the cofactor coelenterazine (25 μM) and glutathione (4 mM), and the microtitre plate is then incubated for a further 3-4 hours. The test substances are then pipetted onto the microtitre plate, and 5 minutes after the transfer of the test substances into the wells of the microtitre plate the plate is transferred into the luminometer, a PAR-1 agonist concentration which corresponds to EC₅₀ is added and the resulting light signal is immediately measured in the luminometer. To distinguish an antagonist substance action from a toxic action, the endogenous purinergic receptor is immediately subsequently activated with agonist (ATP, final concentration 10 μM) and the resulting light signal is measured. The results are shown in Table A:

TABLE A

| Example No. | IC₅₀ [nM] |
|---|---|
| 1 | 177 |
| 6 | 8 |
| 15 | 56 |
| 40 | 3 |
| 48 | 24 |

### 1.b) PAR-1 Receptor Binding Assay

Platelet membranes are incubated with 12 nM [3H]haTRAP and test substance in different concentrations in a buffer (50 mM Tris pH 7.5, 10 mM magnesium chloride, 1 mM EGTA, 0.1% BSA) at room temperature for 80 min. Then the mixture is transferred to a filter plate and washed twice with buffer. After addition of scintillation liquid, the radioactivity on the filter is measured in a beta counter.

### 1.c) Platelet Aggregation in Plasma

To determine the platelet aggregation, blood from healthy volunteers of both genders, who had not received any platelet aggregation-influencing medication for the last ten days, is used. The blood is taken up into monovettes (Sarstedt, Nümbrecht, Germany) which contain, as anticoagulant, sodium citrate 3.8% (1 part of citrate+9 parts of blood). To obtain platelet-rich plasma, the citrated whole blood is centrifuged at 140 g for 20 min

For the aggregation measurements, aliquots of the platelet-rich plasma with increasing concentrations of test substance are incubated at 37° C. for 10 min. Subsequently, aggregation is triggered by addition of a thrombin receptor agonist (TRAP6, SFLLRN) in an aggregometer and determined at 37° C. by means of the turbidimetry method according to Born (Born, G. V. R., Cross M. J., The Aggregation of Blood Platelets; J. Physiol. 1963, 168, 178-195). The SFLLRN concentration leading to maximum aggregation is, if appropriate, determined individually for each donor.

To calculate the inhibitory effect, the maximum increase of light transmission (amplitude of the aggregation curve in %) is determined within 5 minutes after addition of the agonist in the presence and absence of test substance, and the inhibition is calculated. The inhibition curves are used to calculate the concentration which inhibits aggregation by 50%.

### 1.d) Platelet Aggregation in Buffer

To determine platelet aggregation, blood of healthy volunteers of both genders, who had not received any platelet aggregation-influencing medication for the last ten days, is used. The blood is taken up into monovettes (Sarstedt, Nümbrecht, Germany) which contain, as anticoagulant, sodium citrate 3.8% (1 part of citrate+9 parts of blood). To obtain platelet-rich plasma, the citrated whole blood is centrifuged at 140 g for 20 min. One quarter of the volume of ACD buffer (44.8 mM sodium citrate, 20.9 mM citric acid, 74.1 mM glucose and 4 mM potassium chloride) is added to the PRP, and the mixture is centrifuged at 1000 g for 10 minutes. The platelet pellet is resuspended with wash buffer and centrifuged at 1000 g for 10 minutes. The platelets are resuspended in incubation buffer and adjusted to 200 000 cells/W. Prior to the start of the test, calcium chloride and magnesium chloride, final concentration in each case 2 mM (2M stock solution, dilution 1:1000), are added. Note: in the case of ADP-induced aggregation, only calcium chloride is added. The following agonists can be used: TRAP6-trifluoroacetate salt, collagen, human α-thrombin and U-46619. For each donor, the concentration of the agonist is tested.

Test procedure: 96-well microtitre plates are used. The test substance is diluted in DMSO, and 2 ml per well are initially charged. 178 μl of platelet suspension are added, and the mixture is preincubated at room temperature for 10 minutes. 20 μl of agonist are added, and the measurement in the Spectramax, OD 405 nm, is started immediately. Kinetics are determined in 11 measurements of 1 minute each. Between the measurements, the mixture is shaken for 55 seconds.

### 1.e) Platelet Aggregation in Fibrinogen-Depleted Plasma

To determine platelet aggregation, blood of healthy volunteers of both genders, who had not received any platelet aggregation-influencing medication for the last ten days, is used. The blood is taken up into monovettes (Sarstedt, Nümbrecht, Germany) which contain, as anticoagulant, sodium citrate 3.8% (1 part of citrate+9 parts of blood).

Preparation of fibrinogen-depleted plasma: To obtain low-platelet plasma, the citrated whole blood is centrifuged at 140 g for 20 min. The low-platelet plasma is admixed in a ratio of 1:25 with reptilase (Roche Diagnostic, Germany) and inverted cautiously. This is followed by incubation at 37° C. in a water bath for 10 min, followed directly by incubation on ice for 10 min. The plasma/reptilase mixture is centrifuged at 1300 g for 15 min, and the supernatant (fibrinogen-depleted plasma) is obtained.

Platelet isolation: To obtain platelet-rich plasma, the citrated whole blood is centrifuged at 140 g for 20 min. One quarter of the volume of ACD buffer (44.8 mM sodium citrate, 20.9 mM citric acid, 74.1 mM glucose and 4 mM potassium chloride) is added to the PRP, and the mixture is centrifuged at 1300 g for 10 minutes. The platelet pellet is resuspended with wash buffer and centrifuged at 1300 g for 10 minutes. The platelets are resuspended in incubation buffer and adjusted to 400 000 cells/μl, and calcium chloride solution is added with a final concentration of 5 mM (dilution 1/200).

For the aggregation measurements, aliquots (98 μl of fibrinogen-depleted plasma and 80 μl of platelet suspension) are incubated with increasing concentrations of test substance at RT for 10 min. Subsequently, aggregation is triggered by addition of human alpha thrombin in an aggregometer and determined at 37° C. by means of the turbidimetry method according to Born (Born, G. V. R., Cross M. J., The Aggregation of Blood Platelets; J. Physiol. 1963, 168, 178-195). The alpha thrombin concentration which just leads to the maximum aggregation is determined individually for each donor.

To calculate the inhibitory effect, the increase in the maximum light transmission (amplitude of the aggregation curve in %) is determined within 5 minutes after addition of the agonist in the presence and absence of test substance, and the inhibition is calculated. The inhibition curves are used to calculate the concentration which inhibits aggregation by 50%.

### 1.f) Stimulation of Washed Platelets and Analysis in Flow Cytometry

Isolation of washed platelets: Human whole blood is obtained by venipuncture from voluntary donors and transferred to monovettes (Sarstedt, Nümbrecht, Germany) containing sodium citrate as anticoagulant (1 part sodium citrate 3.8%+9 parts whole blood). The monovettes are centrifuged at 90° rotations per minute and 4° C. for a period of 20 minutes (Heraeus Instruments, Germany; Megafuge 1.0RS). The platelet-rich plasma is carefully removed and transferred to a 50 ml Falcon tube. ACD buffer (44 mM sodium citrate, 20.9 mM citric acid, 74.1 mM glucose) is then added to the plasma. The volume of the ACD buffer corresponds to one quarter of the plasma volume.

Centrifuging at 2500 rpm and 4° C. for ten minutes sediments the platelets. Thereafter, the supernatant is cautiously decanted off and discarded. The precipitated platelets are first cautiously resuspended in one millilitre of wash buffer (113 mM sodium chloride, 4 mM disodium hydrogenphosphate, 24 mM sodium dihydrogenphosphate, 4 mM potassium chloride, 0.2 mM ethylene glycol-bis(2-aminoethyl)-N,N,N′,N′-tetraacetic acid, 0.1% glucose) and then made up with wash buffer to a volume which corresponds to that of the amount of plasma. The wash procedure is repeated. The platelets are precipitated by another centrifugation at 2500 rpm and 4° C. for ten minutes and then carefully resuspended in one millilitre of incubation buffer (134 mM sodium chloride, 12 mM sodium hydrogencarbonate, 2.9 mM potassium chloride, 0.34 mM sodium dihydrogencarbonate, 5 mM HEPES, 5 mM glucose, 2 mM calcium chloride and 2 mM magnesium chloride) and adjusted with incubation buffer to a concentration of 300 000 platelets per μl.

Staining and stimulation of the human platelets with human α-thrombin in the presence or absence of a PAR-1 antagonist: The platelet suspension is preincubated with the substance to be tested or the appropriate solvent at 37° C. for 10 minutes (Eppendorf, Germany; Thermomixer Comfort). Platelet activation is triggered by addition of the agonist (0.5 μM or 1 μM α-thrombin; Kordia, the Netherlands, 3281 NIH units/mg; or 30 μg/ml of thrombin receptor activating peptide (TRAP6); Bachem, Switzerland) at 37° and with shaking at 500 rpm. One 50 μl aliquot of removed at each of 0, 1, 2.5, 5, 10 and 15 minutes, and transferred into one millilitre of singly concentrated CellFix™ solution (Becton Dickinson Immunocytometry Systems, USA). To fix the cells, they are incubated in the dark at 4° C. for 30 minutes. The platelets are precipitated by centrifuging at 600 g and 4° C. for ten minutes. The supernatant is discarded and the platelets are resuspended in 400 μl CellWash™ (Becton Dickinson Immunocytometry Systems, USA). One aliquot of 100 μl is transferred to a new FACS tube. 1 μl of the platelet-identifying antibody and 1 μl of the activation state-detecting antibody are made up to a volume of 100 μl with CellWash™. This antibody solution is then added to the platelet suspension and incubated in the dark at 4° C. for 20 minutes. After staining, the reaction volume is increased by addition of a further 400 ml of CellWash™.

A fluorescein isothiocyanate-conjugated antibody directed against human glycoprotein IIb (CD41) (Immunotech Coulter, France; Cat. No. 0649) is used to identify the platelets. With the aid of the phycoerythrin-conjugated antibody directed against human glycoprotein P-selectin (Immunotech Coulter, France; Cat. No. 1759), it is possible to determine the activation state of the platelets. P-Selectin (CD62P) is localized in the α-granules of resting platelets. However, following in vitro or in vivo stimulation, it is translocalized to the external plasma membrane.

Flow cytometry and data evaluation: The samples are analysed in the FACSCalibur™ Flow Cytometry System instrument from Becton Dickinson Immunocytometry Systems, USA, and evaluated and graphically presented with the aid of the CellQuest software, Version 3.3 (Becton Dickinson Immunocytometry Systems, USA). The extent of platelet activation is determined by the percentage of CD62P-positive platelets (CD41-positive events). From each sample, 10 000 CD41-positive events are counted.

The inhibitory effect of the substances to be tested is calculated via the reduction in platelet activation, which relates to the activation by the agonist.

### 1.g) Platelet Aggregation Measurement Using the Parallel-Plate Flow Chamber

To determine platelet activation, blood of healthy volunteers of both genders, who had not received any platelet aggregation-influencing medication for the last ten days, is used. The blood is taken up into monovettes (Sarstedt, Nümbrecht, Germany) which contain, as anticoagulant, sodium citrate 3.8% (1 part citrate+9 parts blood). To obtain platelet-rich plasma, the citrated whole blood is centrifuged at 140 g for 20 min. One quarter of the volume of ACD buffer (44.8 mM sodium citrate, 20.9 mM citric acid, 74.1 mM glucose and 4 mM potassium chloride) is added to the PRP, and the mixture is centrifuged at 1000 g for 10 minutes. The platelet pellet is resuspended in wash buffer and centrifuged at 1000 g for 10 minutes. For the perfusion study, a mixture of 40% erythrocytes and 60% washed platelets (200 000/μl) is prepared and suspended in HEPES-tyrode buffer. Platelet aggregation under flow conditions is measured using the parallel-plate flow chamber (B. Nieswandt et al., EMBO J. 2001, 20, 2120-2130; C. Weeterings, Arterioscler Thromb. Vasc. Biol. 2006, 26, 670-675; J J Sixma, Thromb. Res. 1998, 92, 43-46). Glass slides are wetted with 100 μl of a solution of human α-thrombin (dissolved in Tris buffer) at 4° C. overnight (α-thrombin in different concentrations, e.g. 10 to 50 mg/ml) and then blocked using 2% BSA.

Reconstituted blood is passed over the thrombin-wetted glass slides at a constant flow rate (for example a shear rate of 300/second) for 5 minutes and observed and recorded using a microscope video system. The inhibitory activity of the substances to be tested is determined morphometrically via the reduction of platelet aggregate formation. Alternatively, the inhibition of the platelet activation can be determined by flow cytometry, for example via p-selectin expression (CD62p) (see Method 1.f).

### 2.) Ex Vivo Assay

### 2.a) Platelet Aggregation (Primates, Guinea Pigs)

Awake or anaesthetized guinea pigs or primates are treated orally, intravenously or intraperitoneally with test substances in suitable formulations. As a control, other guinea pigs or primates are treated in an identical manner with the corresponding vehicle. Depending on the mode of administration, blood of the deeply anaesthetized animals is obtained by puncture of the heart or of the aorta for different periods of time. The blood is taken up into monovettes (Sarstedt, Nümbrecht, Germany) which, as anticoagulant, contain sodium citrate 3.8% (1 part citrate solution+9 parts blood). To obtain platelet-rich plasma, the citrated whole blood is centrifuged at 140 g for 20 min.

Aggregation is triggered by addition of a thrombin receptor agonist (TRAP6, SFLLRN, 50 μg/ml; in each experiment, the concentration is determined for each animal species) in an aggregometer and determined by means of the turbidimetry method according to Born (Born, G. V. R., Cross M. J., The Aggregation of Blood Platelets; J. Physiol. 1963, 168, 178-195) at 37° C.

To measure the aggregation, the maximum increase in the light transmission (amplitude of the aggregation curve in %) is determined within 5 minutes after addition of the agonist. The inhibitory effect of the administered test substances in the treated animals is calculated via the reduction in aggregation, based on the mean of the control animals.

### 2.b) Platelet Aggregation and Activation Measurement in the Parallel-Plate Flow Chamber (Primates)

Awake or anaesthetized primates are treated orally, intravenously or intraperitoneally with test substances in suitable formulations. As a control, other animals are treated in an identical manner with the corresponding vehicle. According to the mode of administration, blood is obtained from the animals by venipuncture for different periods of time. The blood is transferred into monovettes (Sarstedt, Nümbrecht, Germany) which, as anticoagulant, contain sodium citrate 3.8% (1 part citrate solution+9 parts blood). Alternatively, non-anticoagulated blood can be taken with neutral monovettes (Sarstedt). In both bases, the blood is admixed with Pefabloc FG (Pentapharm, final concentration 3 mM) to prevent fibrin clot formation.

Citrated whole blood is recalcified before the measurement by adding CaCl₂ solution (final Ca⁺⁺ concentration 5 mM). Non-anticoagulated blood is introduced directly into the parallel-plate flow chamber for measurement. The measurement of platelet activation is conducted by morphometry or flow cytometry in the collagen-coated parallel-plate flow chamber, as described in Method 1.h).

### 3.) In Vivo Assays

### 3.a) Thrombosis Models

The inventive compounds can be studied in thrombosis models in suitable animal species in which thrombin-induced platelet aggregation is mediated via the PAR-1 receptor. Suitable animal species are guinea pigs and, in particular, primates (cf.: Lindahl, A. K., Scarborough, R. M., Naughton, M. A., Harker, L. A., Hanson, S. R., Thromb Haemost 1993, 69, 1196; Cook J J, Sitko G R, Bednar B, Condra C, Mellott M J, Feng D-M, Nutt R F, Shager J A, Gould R J, Connolly T M, Circulation 1995, 91, 2961-2971; Kogushi M, Kobayashi H, Matsuoka T, Suzuki S, Kawahara T, Kajiwara A, Hishinuma I, Circulation 2003, 108 Suppl. 17, IV-280; Derian C K, Damiano B P, Addo M F, Darrow A L, D'Andrea M R, Nedelman M, Zhang H-C, Maryanoff B E, Andrade-Gordon P, J. Pharmacol. Exp. Ther. 2003, 304, 855-861). Alternatively, it is possible to use guinea pigs which have been pretreated with inhibitors of PAR-3 and/or PAR-4 (Leger A J et al., Circulation 2006, 113, 1244-1254), or transgenic PAR-3- and/or PAR-4-knockdown guinea pigs.

### 3.b) Impaired Coagulation and Organ Dysfunction in the Case of Disseminated Intravasal Coagulation (DIC)

The inventive compounds can be tested in models of DIC and/or sepsis in suitable animal species. Suitable animal species are guinea pigs and, in particular, primates, and for the study of endothelium-mediated effects also mice and rats (cf.: Kogushi M, Kobayashi H, Matsuoka T, Suzuki S, Kawahara T, Kajiwara A, Hishinuma I, Circulation 2003, 108 Suppl. 17, IV-280; Derian C K, Damiano B P, Addo M F, Darrow A L, D'Andrea M R, Nedelman M, Zhang H-C, Maryanoff B E, Andrade-Gordon P, J. Pharmacol. Exp. Ther. 2003, 304, 855-861; Kaneider N C et al., Nat Immunol, 2007, 8, 1303-12; Camerer E et al., Blood, 2006, 107, 3912-21; Riewald M et al., J Biol Chem, 2005, 280, 19808-14.). Alternatively, it is possible to use guinea pigs which have been pretreated with inhibitors of PAR-3 and/or PAR-4 (Leger A J et al., Circulation 2006, 113, 1244-1254), or transgenic PAR-3- and/or PAR-4-knockdown guinea pigs.

### 3.b.1) Thrombin-Antithrombin Complexes

Thrombin-antithrombin complexes (referred to hereinafter as “TAT”) are a measure of the thrombin formed endogenously by coagulation activation. TATs are determined via an ELISA assay (Enzygnost TAT micro, Dade-Behring). Plasma is obtained from citrated blood by centrifugation. 50 μl of TAT sample buffer are added to 50 μl of plasma, shaken briefly and incubated at room temperature for 15 min. The samples are filtered with suction, and the well is washed 3 times with wash buffer (300 Owen). Between the wash steps, the plate is tapped to remove any residual wash buffer. Conjugate solution (100 μl) is added and the mixture is incubated at room temperature for 15 min. The samples are filtered with suction, and the well is washed 3 times with wash buffer (300 μl/well). Chromogenic substrate (100 μl/well) is then added, the mixture is incubated in the dark at room temperature for 30 min, stop solution (100 μl/well) is added, and the development of colour at 492 nm is measured (Safire plate reader).

### 3.b.2) Parameters of Organ Dysfunction

Various parameters are determined, which allow conclusions to be drawn with respect to the restriction of function of various internal organs owing to the administration of LPS, and the therapeutic effect of test substances to be estimated. Citrated blood or, if appropriate, lithium heparin blood, is centrifuged, and the plasma is used to determine the parameters. Typically, the following parameters are determined: creatinine, urea, aspartate aminotransferase (AST), alanine aminotransferase (ALT), total bilirubin, lactate dehydrogenase (LDH), total protein, total albumin and fibrinogen. The values give information regarding kidney function, liver function, cardiovascular function and vascular function.

### 3.b.3) Parameters of Inflammation

The extent of the inflammatory reaction triggered by endotoxin can be demonstrated by the rise in inflammation mediators, for example interleukins (1, 6, 8 and 10), tumour necrosis factor alpha or monocyte chemoattractant protein-1, in the plasma. ELISAs or the Luminex system can be used for this purpose.

### 3.c) Antitumour Activity

The inventive compounds can be tested in models of cancer, for example in the human breast cancer model in immunodeficient mice (cf.: S. Even-Ram et. al., Nature Medicine, 1988, 4, 909-914).

### 3.d) Antiangiogenetic Activity

The inventive compounds can be tested in in vitro and in vivo models of angiogenesis (cf.: Caunt et al., Journal of Thrombosis and Haemostasis, 2003, 10, 2097-2102; Haralabopoulos et al., Am J Physiol, 1997, C239-C245; Tsopanoglou et al., JBC, 1999, 274, 23969-23976; Zania et al., JPET, 2006, 318, 246-254).

### 3.e) Blood Pressure- and Pulse-Modulating Activity

The inventive compounds can be tested in in vivo models for their effect on arterial blood pressure and heart rate. To this end, rats (for example Wistar) are provided with implantable radiotelemetry units, and an electronic data acquisition and storage system (Data Sciences, MN, USA) consisting of a chronically implantable transducer/transmitter unit in combination with a liquid-filled catheter is employed. The transmitter is implanted into the peritoneal cavity, and the sensor catheter is positioned in the descending aorta. The inventive compounds can be administered (for example orally or intravenously). Prior to the treatment, the mean arterial blood pressure and the heart rate of the untreated and treated animals are measured, and it is ensured that they are in the range of about 131-142 mmHg and 279-321 beats/minute. PAR-1-activating peptide (SFLLRN; for example doses between 0.1 and 5 mg/kg) is administered intravenously. Blood pressure and heart rate are measured at various time intervals and durations with and without PAR-1-activating peptide and with and without one of the inventive compounds (cf.: Cicala C et al., The FASEB Journal, 2001, 15, 1433-5; Stasch J P et al., British Journal of Pharmacology 2002, 135, 344-355).

### 4.) Determination of the Solubility

### Preparation of the Starting Solution (Original Solution):

At least 1.5 mg of the test substance are weighed out accurately into a wide-mouth 10 mm screw V-vial (from Glastechnik Gräfenroda GmbH, Art. No. 8004-WM-HN150 with fitting screw cap and septum, DMSO is added to a concentration of 50 mg/ml and the vial is vortexed for 30 minutes.

### Preparation of the Calibration Solutions:

The pipetting steps necessary are effected in 1.2 ml 96-well deep well plates (DWP) with the aid of a liquid-handling robot. The solvent used is a mixture of acetonitrile/water 8:2.

Preparation of the starting solution of calibration solutions (stock solution): 833 μl of the solvent mixture are added to 10 μl of the original solution (concentration=600 mg/ml), and the mixture is homogenized. 1:100 dilutions in separate DWPs are prepared from each test substance, and these are homogenized in turn.

Calibration solution 5 (600 ng/ml): 270 μl of the solvent mixture are added to 30 μl of the stock solution, and the mixture is homogenized.

Calibration solution 4 (60 ng/ml): 270 μl of the solvent mixture are added to 30 μl of the calibration solution 5, and the mixture is homogenized.

Calibration solution 3 (12 ng/ml): 400 μl of the solvent mixture are added to 100 μl of the calibration solution 4, and the mixture is homogenized.

Calibration solution 2 (1.2 ng/ml): 270 μl of the solvent mixture are added to 30 μl of the calibration solution 3, and the mixture is homogenized.

Calibration solution 1 (0.6 ng/ml): 150 μl of the solvent mixture are added to 150 μl of the calibration solution 2, and the mixture is homogenized.

### Preparation of the Sample Solutions:

The pipetting steps necessary are effected in 1.2 ml 96-well DWPs with the aid of a liquid-handling robot. 1000 μl of PBS buffer pH 6.5 are added to 10.1 μl of the stock solution. (PBS buffer pH 6.5: 61.86 g sodium chloride, 39.54 g sodium dihydrogen phosphate and 83.35 g 1 N sodium hydroxide solution are weighed into a 1 litre standard flask and made up to the mark with water, and the mixture is stirred for about 1 hour. 500 ml of this solution are introduced into a 5 litre standard flask and made up to the mark with water. The pH is adjusted to 6.5 using 1 N sodium hydroxide solution.)

### Procedure:

The pipetting steps necessary are effected in 1.2 ml 96-well DWPs with the aid of a liquid-handling robot. The sample solutions prepared in this manner are shaken at 1400 rpm and at 20° C. using a variable temperature shaker for 24 hours. 180 μl are taken from each of these solutions and transferred into Beckman Polyallomer centrifuge tubes. These solutions are centrifuged at about 223 000×g for 1 hour. From each sample solution, 100 μl of the supernatant are removed and diluted 1:10 and 1:1000 with PBS buffer 6.5.

### Analysis:

The samples are analysed by means of HPLC/MS-MS. The test compound is quantified by means of a five-point calibration curve. The solubility is expressed in mg/l. Analysis sequence: 1) blank (solvent mixture); 2) calibration solution 0.6 ng/ml; 3) calibration solution 1.2 ng/ml; 4) calibration solution 12 ng/ml; 5) calibration solution 60 ng/ml; 6) calibration solution 600 ng/ml; 7) blank (solvent mixture); 8) sample solution 1:1000; 9) sample solution 1:10.

### HPLC/MS-MS Method:

HPLC: Agilent 1100, quat. pump (G1311A), autosampler CTC HTS PAL, degasser (G1322A) and column thermostat (G1316A); column: Oasis HLB 20 mm×2.1 mm, 25μ; temperature: 40° C.; eluent A: water+0.5 ml of formic acid/1; eluent B: acetonitrile+0.5 ml of formic acid/1; flow rate: 2.5 ml/min; stop time 1.5 min; gradient: 0 min 95% A, 5% B; ramp: 0-0.5 min 5% A, 95% B; 0.5-0.84 min 5% A, 95% B; ramp: 0.84-0.85 min 95% A, 5% B; 0.85-1.5 min 95% A, 5% B.

MS/MS: WATERS Quattro Micro Tandem MS/MS; Z-Spray API interface; HPLC-MS inlet splitter 1:20; measurement in the ESI mode.

### C) WORKING EXAMPLES OF PHARMACEUTICAL COMPOSITIONS

The inventive substances can be converted to pharmaceutical preparations as follows:

### Tablet:

### Composition:

100 mg of the compound of Example 1, 50 mg of lactose (monohydrate), 50 mg of maize starch, 10 mg of polyvinylpyrrolidone (PVP 25) (from BASF, Germany) and 2 mg of magnesium stearate.

Tablet weight 212 mg. Diameter 8 mm, radius of curvature 12 mm.

### Production:

The mixture of the compound of Example 1, lactose and starch is granulated with a 5% solution (m/m) of the PVP in water. The granules are dried and then mixed with the magnesium stearate for 5 min. This mixture is compressed in a conventional tablet press (see above for tablet format).

Oral suspension:

### Composition:

1000 mg of the compound of Example 1, 1000 mg of ethanol (96%), 400 mg of Rhodigel (xanthan gum) (from FMC, USA) and 99 g of water.

A single dose of 100 mg of the inventive compound corresponds to 10 ml of oral suspension.

### Production:

The Rhodigel is suspended in ethanol, and the compound of Example 1 is added to the suspension. The water is added while stirring. The mixture is stirred for approx. 6 h until the Rhodigel has finished swelling.

### Intravenously Administrable Solution:

### Composition:

1 mg of the compound of Example 1, 15 g of polyethylene glycol 400 and 250 g of water for injections.

### Production:

The compound of Example 1 is dissolved together with polyethylene glycol 400 by stirring in the water. The solution is sterile-filtered (pore diameter 0.22 μm) and dispensed under aseptic conditions into heat-sterilized infusion bottles. The latter are closed with infusion stoppers and crimped caps.

1. A compound of the formula 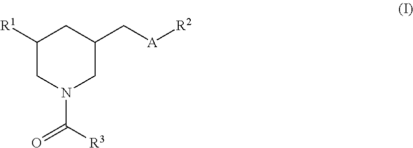
in which
A is an oxygen atom, a sulphur atom, —NR⁴—, —S(O═)— or —S(═O)₂—,
where
R⁴ is hydrogen or C₁-C₃-alkyl, or
R² and R⁴ together with the nitrogen atom to which they are bonded form a 5- or 6-membered heterocycle,
in which the heterocycle may be substituted by 1 to 3 substituents selected independently from the group consisting of halogen, cyano, hydroxyl, amino, C₁-C₄-alkyl, C₁-C₄-alkoxy and C₁-C₄-alkylamino, or
A is a group of the formula 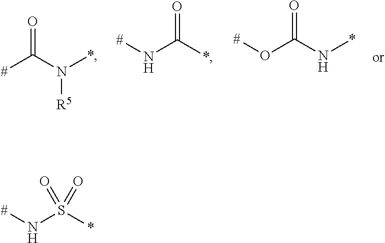
where
# is the attachment site to the piperidine ring,
* is the attachment site of R², and
R⁵ is hydrogen or C₁-C₃-alkyl,
R¹ is phenyl,
where phenyl may be substituted by 1 to 3 substituents selected independently from the group consisting of monofluoromethyl, difluoromethyl, trifluoromethyl, monofluoromethoxy, difluoromethoxy, trifluoromethoxy, monofluoromethylsulphanyl, difluoromethylsulphanyl, trifluoromethylsulphanyl, methylsulphonyl, C₁-C₄-alkyl, C₁-C₄-alkoxy and C₁-C₄-alkoxycarbonyl,
R² is C₁-C₆-alkyl, C₃-C₆-cycloalkyl, 4- to 6-membered heterocyclyl, phenyl or 5- or 6-membered heteroaryl,
where cycloalkyl, heterocyclyl, phenyl and heteroaryl may be substituted by 1 to 3 substituents selected independently from the group consisting of halogen, cyano, hydroxyl, amino, monofluoromethyl, difluoromethyl, trifluoromethyl, monofluoromethoxy, difluoromethoxy, trifluoromethoxy, monofluoromethylsulphanyl, difluoromethylsulphanyl, trifluoromethylsulphanyl, C₁-C₄-alkyl, C₁-C₄-alkoxy, C₁-C₆-alkylamino and phenyl,
in which phenyl may be substituted by 1 to 3 substituents selected independently from the group consisting of halogen and trifluoromethyl, and
where C₁-C₆-alkyl may be substituted by one substituent selected from the group consisting of C₁-C₄-alkoxy, C₁-C₄-alkylsulphonyl, C₃-C₆-cycloalkyl and phenyl,
in which cycloalkyl and phenyl may be substituted by 1 to 3 substituents selected independently from the group consisting of halogen, cyano, monofluoromethyl, difluoromethyl, trifluoromethyl, monofluoromethoxy, difluoromethoxy, trifluoromethoxy, C₁-C₄-alkyl and C₁-C₄-alkoxy,
R³ is C₁-C₆-alkyl, C₁-C₆-alkoxy, C₁-C₆-alkylamino, C₃-C₇-cycloalkyl, 4- to 7-membered heterocyclyl, phenyl, 5- or 6-membered heteroaryl, C₃-C₇-cycloalkyloxy, C₃-C₇-cycloalkylamino, 4- to 7-membered heterocyclylamino, phenylamino or 5- or 6-membered heteroarylamino,
where alkyl, C₂-C₆-alkoxy and alkylamino may be substituted by one substituent selected from the group consisting of halogen, hydroxyl, amino, cyano, C₁-C₄-alkoxy, C₁-C₄-alkoxycarbonyl, C₃-C₇-cycloalkyl, 4- to 6-membered heterocyclyl, phenyl and 5- or 6-membered heteroaryl, and
where cycloalkyl, heterocyclyl, phenyl, heteroaryl, cycloalkyloxy, cycloalkylamino, heterocyclylamino, phenylamino and heteroarylamino may be substituted by 1 to 3 substituents selected independently from the group consisting of halogen, cyano, oxo, hydroxyl, amino, monofluoromethyl, difluoromethyl, trifluoromethyl, monofluoromethoxy, difluoromethoxy, trifluoromethoxy, monofluoromethylsulphanyl, difluoromethylsulphanyl, trifluoromethylsulphanyl, hydroxycarbonyl, aminocarbonyl, C₁-C₄-alkyl, C₁-C₄-alkoxy, C₁-C₆-alkylamino, C₁-C₄-alkoxycarbonyl, C₁-C₄-alkylaminocarbonyl and cyclopropyl,
in which alkyl may be substituted by one hydroxyl substituent,
or one of its salts, its solvates or the solvates of its salts.

2. A compound according to claim 1, wherein
A is an oxygen atom, a sulphur atom, —NR⁴—, —S(O═)— or —S(═O)₂—,
where
R⁴ is hydrogen, methyl or ethyl, or
R² and R⁴ together with the nitrogen atom to which they are bonded form a 3-hydroxypyrrolidin-1-yl, or
A is a group of the formula 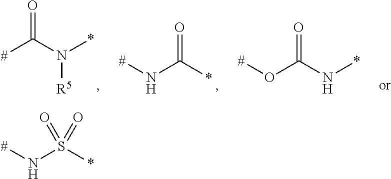
where
# is the attachment site to the piperidine ring,
* is the attachment site of R², and
R⁵ is hydrogen, methyl or ethyl,
R¹ is phenyl,
where phenyl is substituted by 1 to 2 substituents selected independently from the group consisting of trifluoromethyl, trifluoromethoxy, methyl, ethyl and methoxy,
R² is methyl, ethyl, propyl, isopropyl, 2-methylprop-1-yl, tert-butyl, cyclopropyl, cyclobutyl, cyclopentyl, cyclohexyl, tetrahydro-2H-pyran-4-yl or phenyl,
where phenyl may be substituted by 1 to 2 substituents selected independently from the group consisting of halogen, cyano, methyl, ethyl, methoxy and ethoxy, and
where methyl and ethyl may be substituted by one substituent selected from the group consisting of methoxy, methylsulphonyl and phenyl,
R³ is morpholin-4-yl, 1,1-dioxidothiomorpholin-4-yl, 3-hydroxyazetidin-1-yl, 3-hydroxypyrrolidin-1-yl, 4-cyanopiperidin-2-yl or 4-hydroxypiperidin-1-yl,
or one of its salts, its solvates or the solvates of its salts.

3. A compound according to claim 1, wherein
A is a group of the formula 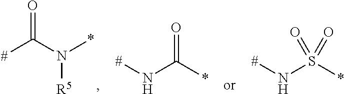
where
# is the attachment site to the piperidine ring,
* is the attachment site to R², and
R⁵ is hydrogen, methyl or ethyl,
R¹ is phenyl,
where phenyl is substituted by 1 to 2 substituents selected independently from the group consisting of trifluoromethyl, trifluoromethoxy, methyl, ethyl and methoxy,
R² is methyl, ethyl, propyl, isopropyl, 2-methylprop-1-yl, tert-butyl, cyclopropyl, cyclobutyl, cyclopentyl, cyclohexyl, tetrahydro-2H-pyran-4-yl or phenyl,
where phenyl may be substituted by 1 to 2 substituents selected independently from the group consisting of halogen, cyano, methyl, ethyl, methoxy and ethoxy, and
where methyl and ethyl may be substituted by one substituent selected from the group consisting of methoxy, methylsulphonyl and phenyl,
R³ is morpholin-4-yl, thiomorpholin-4-yl, 1,1-dioxidothiomorpholin-4-yl, 3-hydroxyazetidin-1-yl, 3-hydroxypyrrolidin-1-yl, 4-cyanopiperidin-1-yl or 4-hydroxypiperidin-1-yl,
or one of its salts, its solvates or the solvates of its salts.

4. A compound according to claim 1, wherein the —R¹ and —CH₂-A-R² substituents are in cis-positions to one another.

5. A process for preparing a compound of the formula (I) or one of its salts, its solvates or the solvates of its salts according to claim 1, wherein either
[A] a compound of the formula 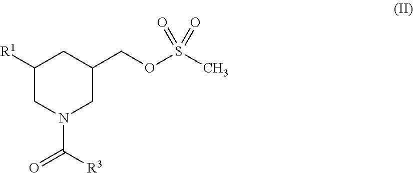
in which
R¹ and R³ are each as defined in claim 1
is reacted with a compound of the formula
H-A-R² (III),
in which
R² is as defined in claim 1
A is an oxygen atom, an sulphur atom or —NR⁴—, and
R⁴ is as defined in claim 1
to give compounds of the formula 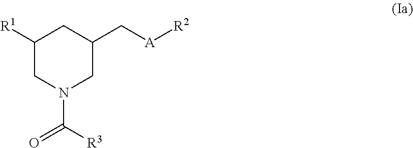
in which
A is an oxygen atom, a sulphur atom or —NR⁴—, and
R¹, R², R³ and R⁴ are each as defined in claim 1, or
[B] compounds of the formula (I) in which A is sulphur atom and R′, R² and R³ are each as defined above are reacted with an oxidizing agent
to give compounds of the formula 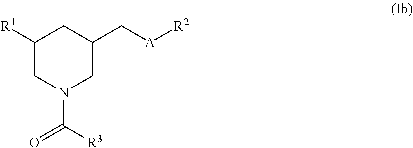
in which
A is —S(═O)— or —S(═O)₂—, and
R¹, R² and R³ are each as defined in claim 1, or
[C] a compound of the formula 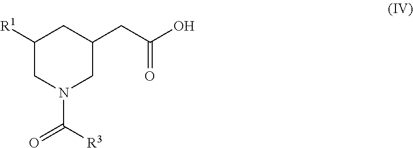
in which
R¹ and R³ are each as defined in claim 1,
is reacted with a compound of the formula 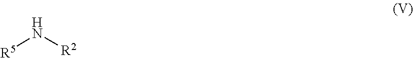
in which
R² and R⁵ are each as defined in claim 1
To give a compound of the formula 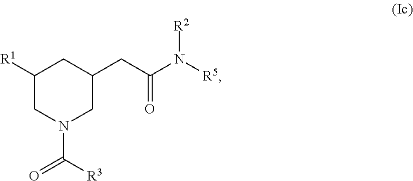
in which
R¹, R², R³ and R⁵ are each as defined in claim 1, or
[D] a compound of the formula 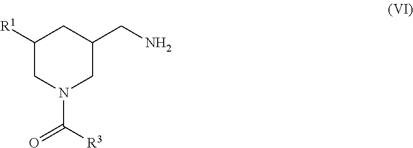
in which
R¹ and R³ are each as defined in claim 1
are reacted with compounds of the formula 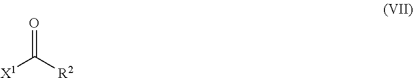
in which
R² is as defined in claim 1 and
X¹ is halogen, preferably bromine or chlorine, or hydroxyl
to give compounds of the formula 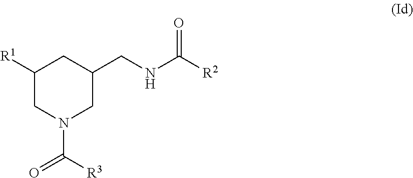
in which
R¹, R² and R³ are each as defined in claim 1, or
[E] compounds of the formula (VI) are reacted with compounds of the formula 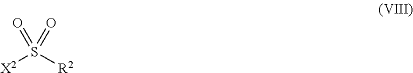
in which
R² is as defined in claim 1 and
X² is halogen, preferably bromine or chlorine,
to give compounds of the formula 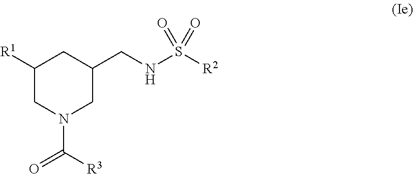
in which
R¹, R² and R³ are each as defined in claim 1, or
[F] compounds of the formula 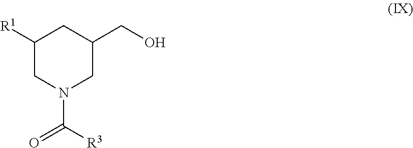
in which
R¹ and R³ are each as defined in claim 1
are reacted with compounds of the formula
R²—N— —O (X)
in which
R² is as defined in claim 1,
to give compounds of the formula 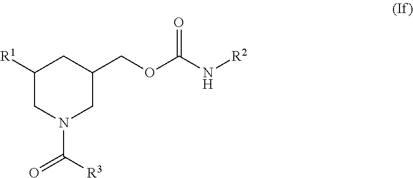
in which
R¹, R² and R³ are as defined in claim 1.

6. (canceled)

7. (canceled)

8. (canceled)

9. (canceled)

10. A pharmaceutical composition comprising a compound according to claim 1 in combination with an inert, non-toxic, pharmaceutically acceptable excipient.

11. A pharmaceutical composition comprising a compound according to claim 1 in combination with a further active ingredient.

12. (canceled)

13. A method for the treatment and/or prophylaxis of a thromboembolic disorder comprising administering to a human or animal in need thereof an anticoagulatory amount of a compound according to claim 1.

14. A method for the prevention of blood coagulation in vitro, comprising adding to blood ex vivo an anticoagulatory amount of a compound according to claim 1.

15. A method for the treatment and/or prophylaxis of a cardiovascular disorder comprising administering to a human or animal in need thereof an effective amount of a compound according to claim 1.

16. A method for the treatment and/or prophylaxis of a tumour disorder comprising administering to a human or animal in need thereof an effective amount of a compound according to claim 1.

17. A method for the treatment and/or prophylaxis of a thromboembolic disorder comprising administering to a human or animal in need thereof an anticoagulatory amount of a pharmaceutical composition according to claim 10.

18. A method for the treatment and/or prophylaxis of a cardiovascular disorder comprising administering to a human or animal in need thereof an anticoagulatory amount of a pharmaceutical composition according to claim 10.

19. A method for the treatment and/or prophylaxis of a tumour disorder comprising administering to a human or animal in need thereof an anticoagulatory amount of a pharmaceutical composition according to claim 10.
